SEQUENCE LISTING (20 sequences)

SEQ ID NO: 1 - DNA, 22 bp, Homo sapiens
tggcctggagcaacaaatctga

SEQ ID NO: 2 - DNA, 22 bp, Homo sapiens
accggacctcgttgtttagact

SEQ ID NO: 3 - protein, 120 aa, Artificial Sequence
  Synthesized
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 4 - protein, 107 aa, Artificial Sequence
  Synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 486 aa, Artificial Sequence
  Synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 122 aa, Artificial Sequence
  Synthesized
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 7 - protein, 112 aa, Artificial Sequence
  Synthesized
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 8 - protein, 493 aa, Artificial Sequence
  Synthesized
MALPVTALLLPLALLLHAARPDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 4 aa, Artificial Sequence
  Synthesized
QEED

SEQ ID NO: 10 - protein, 4 aa, Artificial Sequence
  Synthesized
EEEE

SEQ ID NO: 11 - protein, 4 aa, Artificial Sequence
  Synthesized
SEEE

SEQ ID NO: 12 - protein, 42 aa, Artificial Sequence
  Synthesized
KASRKKAAAAAKSPFASPASSAQEEDASSCRAPSEEEGSCEL

SEQ ID NO: 13 - protein, 42 aa, Artificial Sequence
  Synthesized
KRGRKKLLYIFKQPFMRPVQTTQEEDGCSCRFPEEEEGGCEL

SEQ ID NO: 14 - protein, 24 aa, Artificial Sequence
  Synthesized
TTTPAPRPPTPAPTIASQPLSLRP

SEQ ID NO: 15 - protein, 45 aa, Artificial Sequence
  Synthesized
EACRPAAGGAVHTRGLDFACDIYIWAPLAGTCGVLLLSLVITLYC

SEQ ID NO: 16 - protein, 112 aa, Artificial Sequence
  Synthesized
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 354 aa, Artificial Sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 18 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 19 - DNA, 4627 bp, Homo sapiens
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 20 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

(19) United States
(12) Patent Application Publication
(10) Pub. No.: US 2023/0190780 A1
(43) Pub. Date: Jun. 22, 2023

(54) METHODS FOR IMMUNOTHERAPY

(71) Applicant: Precision BioSciences, Inc., Durham, NC (US)

(72) Inventors: Christopher Ryan Heery, Durham, NC (US); Daniel H. Fowler, Bethesda, MD (US); Alan F. List, Tampa, FL (US); Aaron Martin, Carrboro, NC (US); Daniel T. MacLeod, Durham, NC (US); Derek Jantz, Durham, NC (US)

(73) Assignee: Precision BioSciences, Inc., Durham, NC (US)

(21) Appl. No.: 17/925,277

(22) PCT Filed: May 14, 2021
(86) PCT No.: PCT/US2021/032591

§ 371 Date: Nov. 14, 2022

Related U.S. Application Data
(60) Provisional application No. 63/061,736, filed on Aug. 5, 2020.
(60) Provisional application No. 63/025,772, filed on May 15, 2020.

(51) Int. Cl. A61K 31/7056; A61K 31/675; C07K 16/28; C07K 14/725; A61K 31/436; A61P 35/00

(52) U.S. Cl. CPC A61K 31/7056; A61K 31/675; C07K 16/2893; C07K 14/7051; A61K 31/436; A61P 35/00; A61K 2039/545

(57) ABSTRACT

The present invention encompasses methods for reducing the number of target cells in a subject, such as cancer cells. The methods include administration of genetically-modified human immune cells expressing a chimeric antigen receptor or exogenous T cell receptor, which have specificity for an antigen on the target cells Administration of the genetically-modified immune cells can be preceded by the administration of a lymphodepletion region and/or an immunosuppression regimen, to improve efficacy of the therapy and persistence of the cells in vivo.

### FIELD OF THE INVENTION

The invention relates to the field of immunotherapy. In particular, the invention relates to allogeneic or autologous cellular immunotherapy.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on May 14, 2021, is named P109070049WO00-SEQ-EPG, and is 28,777 bytes in size.

### BACKGROUND OF THE INVENTION

Immune cell adoptive immunotherapy is a promising approach for treatment of various diseases, including cancer. The immunotherapy treatment methods disclosed herein utilize isolated human immune cells that have been genetically-modified to enhance their specificity for a specific antigen on target cells. Genetic modification may involve the expression of a chimeric antigen receptor or an exogenous T cell receptor to graft antigen specificity onto the immune cell. In contrast to exogenous T cell receptors, chimeric antigen receptors derive their specificity from the variable domains of a monoclonal antibody. Thus, immune cells expressing chimeric antigen receptors (such as CAR T cells) induce immunoreactivity in a major histocompatibility complex non-restricted manner. T cell adoptive immunotherapy has been utilized as a clinical therapy for a number of cancers, including B cell malignancies (e.g., acute lymphoblastic leukemia, B cell non-Hodgkin lymphoma, acute myeloid leukemia, and chronic lymphocytic leukemia), multiple myeloma, neuroblastoma, glioblastoma, advanced gliomas, ovarian cancer, mesothelioma, melanoma, prostate cancer, pancreatic cancer, and others.

Clinical trials have largely focused on the use of autologous CAR T cells, wherein a patient's T cells are isolated, genetically-modified to incorporate a chimeric antigen receptor, and then re-infused into the same patient. Despite its potential usefulness as a cancer treatment, adoptive immunotherapy with autologous CAR T cells has been limited, in part, by safety, cost, and logistical concerns. To address these problems, efforts have been undertaken to apply the same technology into an off-the-shelf product produced from healthy donor's peripheral blood mononuclear cells (PBMC). However, these off-the-shelf allogeneic CAR T cells have a new limitation, the expression of the endogenous T cell receptor on the cell surface. Allogeneic CAR T cells expressing their endogenous T cell receptor may recognize major and minor histocompatibility antigens present on normal healthy cells of the patient following administration, which can lead to the development of graft-versus-host-disease (GVHD). Additionally, the retained expression of the Major Histocompatibility Complex (MHC) provides an opportunity for the host T cells to recognize and reject the allogeneic CAR T cells through host versus graft (HVG) allogeneic rejection. This results in a potential limitation of the expansion and persistence of allogeneic off-the-shelf CAR T cells. Potential solutions to address this issue include gene knock-out of beta-2-micrglobulin (B2M), gene suppression (knock-down) of B2M, and alternative dosing strategies to overcome the limitations of potential rejection. Those dosing strategies may include repeat dosing with different donor products after repeat lymphodepletion, lymphodepletion strategies that extend the duration of suppression of host immunity, and potentially others.

Currently, prior to CAR T cell therapy, patients are pre-treated with a round of chemotherapy for purposes of lymphodepletion. This is typically carried out 3 days to 1 week prior to injection with the CAR T cells. In terms of autologous cell therapy, this is generally sufficient to eliminate enough of the host lymphocytes to generate supportive cytokines, make space for the incoming CAR T cells to benefit from the microenvironment of the host, and promote expansion of the incoming CAR T cells (see Hay et al., Drugs (2017) 77(3): 237-245).

The applicant has developed an “off the shelf” CAR T cell platform, prepared using T cells from a third party, healthy donor, that knocks out the expression of the endogenous T cell receptor (e.g., an alpha/beta T cell receptor) and does not cause GVHD upon administration, based on preclinical models and current clinical trial data. Notably, the CAR is inserted into the T cell receptor alpha subunit (TRAC) locus. The result is that any cell expressing the CAR does not have the TRAC locus intact and therefore does not express an active T cell receptor. These CAR T products are produced, vialed, and shipped to treatment facilities so that they are available to patients as soon as necessary. However, the current version of the CAR T cell platform being evaluated in the clinic retains the expression of MHC, and it is therefore subject to the potential allogeneic HVG rejection process described. Therefore, there may be a need to identify novel methods to delay rejection of the allogeneic CAR Ts so that all cancer cells can be killed.

### SUMMARY OF THE INVENTION

The present invention provides methods for reducing the number of target cells in a subject. In one aspect, the invention provides methods for reducing the number of target cells in a subject that comprises administering to the subject a lymphodepletion regimen that includes administering one or more effective doses of a purine analog (e.g., pentostatin) and one or more effective doses of a compound capable of inducing interstrand cross-links within DNA (such as cisplatin, mitomycin C, carmustine, psoralen or nitrogen mustard-derived alkylating agents like cyclophosphamide, ifosfamide, chlorambucil, uramustine, melphalan, and bendamustine), and administering to the subject an effective dose of a pharmaceutical composition comprising a population of human immune cells, wherein a plurality of the human immune cells are genetically-modified human immune cells that express a chimeric antigen receptor (CAR) or an exogenous T cell receptor (TCR). The lymphodepletion regimen is administered prior to administration of the pharmaceutical composition, and the CAR or the exogenous TCR comprises an extracellular ligand-binding domain having specificity for an antigen on the target cells.

In certain embodiments of the method, the compound capable of inducing interstrand cross-links within DNA is an alkylating agent (e.g., cyclophosphamide). In some embodiments, the lymphodepletion regimen comprises administering an alkylating agent (e.g., cyclophosphamide) at a dose of about 50 to about 500 mg/day, about 100 to about 300 mg/day, about 50 to about 300 mg/day, about 100 to about 500 mg/day, or about 150 to about 250 mg/day. In some of these embodiments, the lymphodepletion regimen comprises administering an alkylating agent (e.g., cyclophosphamide) at a dose of about 50, about 100, about 150, about 175, about 180, about 185, about 190, about 195, about 200, about 205, about 210, about 215, about 220, about 225, about 250, about 300, about 350, about 400, about 450, or about 500 mg/day. In particular embodiments, the lymphodepletion regimen comprises administering an alkylating agent (e.g., cyclophosphamide) at a dose of about 200 mg/day.

In certain embodiments, the lymphodepletion regimen comprises administering an alkylating agent (e.g., cyclophosphamide) once daily starting 11 days prior to administration of the pharmaceutical composition and ending 1 day prior to administration of the pharmaceutical composition, starting 9 days prior to administration of the pharmaceutical composition and ending 1 day prior to administration of the pharmaceutical composition, starting 7 days prior to administration of the pharmaceutical composition and ending 1 day prior to administration of the pharmaceutical composition, starting 11 days prior to administration of the pharmaceutical composition and ending 2 days prior to administration of the pharmaceutical composition, starting 9 days prior to administration of the pharmaceutical composition and ending 2 days prior to administration of the pharmaceutical composition, starting 7 days prior to administration of the pharmaceutical composition and ending 2 days prior to administration of the pharmaceutical composition, starting 11 days prior to administration of the pharmaceutical composition and ending 3 days prior to administration of the pharmaceutical composition, starting 9 days prior to administration of the pharmaceutical composition and ending 3 days prior to administration of the pharmaceutical composition, or starting 7 days prior to administration of the pharmaceutical composition and ending 3 days prior to administration of the pharmaceutical composition.

In some embodiments, the lymphodepletion regimen comprises administering an alkylating agent (e.g., cyclophosphamide) once daily starting 11 days prior to administration of the pharmaceutical composition and ending 10 days, 9 days, 8 days, 7 days, 6 days, 5 days, 4 days, 3 days, 2 days, or 1 day prior to administration of the pharmaceutical composition. In other embodiments, the lymphodepletion regimen comprises administering an alkylating agent (e.g., cyclophosphamide) once daily starting 10 days prior to administration of the pharmaceutical composition and ending 9 days, 8 days, 7 days, 6 days, 5 days, 4 days, 3 days, 2 days, or 1 day prior to administration of the pharmaceutical composition. In still other embodiments, the lymphodepletion regimen comprises administering an alkylating agent (e.g., cyclophosphamide) once daily starting 9 days prior to administration of the pharmaceutical composition and ending 8 days, 7 days, 6 days, 5 days, 4 days, 3 days, 2 days, or 1 day prior to administration of the pharmaceutical composition. In yet other embodiments, the lymphodepletion regimen comprises administering an alkylating agent (e.g., cyclophosphamide) once daily starting 8 days prior to administration of the pharmaceutical composition and ending 7 days, 6 days, 5 days, 4 days, 3 days, 2 days, or 1 day prior to administration of the pharmaceutical composition. In other embodiments, the lymphodepletion regimen comprises administering an alkylating agent (e.g., cyclophosphamide) once daily starting 7 days prior to administration of the pharmaceutical composition and ending 6 days, 5 days, 4 days, 3 days, 2 days, or 1 day prior to administration of the pharmaceutical composition. In still other embodiments, the lymphodepletion regimen comprises administering an alkylating agent (e.g., cyclophosphamide) once daily starting 6 days prior to administration of the pharmaceutical composition and ending 5 days, 4 days, 3 days, 2 days, or 1 day prior to administration of the pharmaceutical composition. In yet other embodiments, the lymphodepletion regimen comprises administering an alkylating agent (e.g., cyclophosphamide) once daily starting 5 days prior to administration of the pharmaceutical composition and ending 4 days, 3 days, 2 days, or 1 day prior to administration of the pharmaceutical composition. In other embodiments, the lymphodepletion regimen comprises administering an alkylating agent (e.g., cyclophosphamide) once daily starting 4 days prior to administration of the pharmaceutical composition and ending 3 days, 2 days, or 1 day prior to administration of the pharmaceutical composition. In still other embodiments, the lymphodepletion regimen comprises administering an alkylating agent (e.g., cyclophosphamide) once daily starting 3 days prior to administration of the pharmaceutical composition and ending 2 days or 1 day prior to administration of the pharmaceutical composition. In yet other embodiments, the lymphodepletion regimen comprises administering an alkylating agent (e.g., cyclophosphamide) once daily starting 2 days prior to administration of the pharmaceutical composition and ending 1 day prior to administration of the pharmaceutical composition.

In particular embodiments, the lymphodepletion regimen comprises administering an alkylating agent (e.g., cyclophosphamide) once daily starting 7 days prior to administration of the pharmaceutical composition and ending 1 day prior to administration of the pharmaceutical composition.

In some embodiments, the alkylating agent (e.g., cyclophosphamide) is administered orally.

In some of these embodiments, the lymphodepletion regimen comprises administering an alkylating agent (e.g., cyclophosphamide) once daily at a dose of about 50 to about 500 mg/day, about 100 to about 300 mg/day, about 50 to about 300 mg/day, about 100 to about 500 mg/day, or about 150 to about 250 mg/day, including but not limited to about 50, about 100, about 150, about 175, about 180, about 185, about 190, about 195, about 200, about 205, about 210, about 215, about 220, about 225, about 250, about 300, about 350, about 400, about 450, or about 500 mg/day starting 11 days, 10 days, 9 days, 8 day, 7 days, 6 days, 5 days, 4 days, 3 days, or 2 days prior to administration of the pharmaceutical composition and ending 1 day prior to administration of the pharmaceutical composition, starting 11 days, 10 days, 9 days, 8 day, 7 days, 6 days, 5 days, 4 days, or 3 days prior to administration of the pharmaceutical composition and ending 2 days prior to administration of the pharmaceutical composition, or starting 11 days, 10 days, 9 days, 8 day, 7 days, 6 days, 5 days, or 4 days prior to administration of the pharmaceutical composition and ending 3 days prior to administration of the pharmaceutical composition. In particular embodiments, the lymphodepletion regimen comprises administering about 200 mg of cyclophosphamide orally once daily starting 7 days prior to administration of the pharmaceutical composition and ending 1 day prior to administration of the pharmaceutical composition.

In some embodiments, the lymphodepletion regimen comprises administering a purine analog (e.g., pentostatin) at a dose of about 1 to about 30 mg/m²/day, about 1 to about 20 mg/m²/day, about 1 to about 10 mg/m²/day, about 2 to about 20 mg/m²/day, or about 2 to about 10 mg/m²/day. In some of these embodiments, the lymphodepletion regimen comprises administering a purine analog (e.g., pentostatin) at a dose of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mg/m²/day. In particular embodiments, the lymphodepletion regimen comprises administering a purine analog (e.g., pentostatin) at a dose of about 4 mg/m²/day.

In certain embodiments, the lymphodepletion regimen comprises administering a purine analog (e.g., pentostatin) once daily starting 12 days prior to administration of the pharmaceutical composition and ending 1 day prior to administration of the pharmaceutical composition, starting 9 days prior to administration of the pharmaceutical composition and ending 1 day prior to administration of the pharmaceutical composition, starting 7 days prior to administration of the pharmaceutical composition and ending 1 day prior to administration of the pharmaceutical composition, starting 12 days prior to administration of the pharmaceutical composition and ending 2 days prior to administration of the pharmaceutical composition, starting 9 days and ending 2 days, starting 7 days prior to administration of the pharmaceutical composition and ending 2 days prior to administration of the pharmaceutical composition, starting 12 days and ending 3 days, starting 9 days prior to administration of the pharmaceutical composition and ending 3 days prior to administration of the pharmaceutical composition, starting 7 days prior to administration of the pharmaceutical composition and ending 3 days prior to administration of the pharmaceutical composition, or starting 9 prior to administration of the pharmaceutical composition days and ending 3 days prior to administration of the pharmaceutical composition, prior to administration of the pharmaceutical composition.

In some of these embodiments, the lymphodepletion regimen comprises administering a purine analog (e.g., pentostatin) once daily starting 12 days prior to administration of the pharmaceutical composition and ending 11 days, 10 days, 9 days, 8 days, 7 days, 6 days, 5 days, 4 days, 3 days, 2 days, or 1 day prior to administration of the pharmaceutical composition. In some of these embodiments, the lymphodepletion regimen comprises administering a purine analog (e.g., pentostatin) once daily starting 11 days prior to administration of the pharmaceutical composition and ending 10 days, 9 days, 8 days, 7 days, 6 days, 5 days, 4 days, 3 days, 2 days, or 1 day prior to administration of the pharmaceutical composition. In other embodiments, the lymphodepletion regimen comprises administering a purine analog (e.g., pentostatin) once daily starting 10 days prior to administration of the pharmaceutical composition and ending 9 days, 8 days, 7 days, 6 days, 5 days, 4 days, 3 days, 2 days, or 1 day prior to administration of the pharmaceutical composition. In still other embodiments, the lymphodepletion regimen comprises administering a purine analog (e.g., pentostatin) once daily starting 9 days prior to administration of the pharmaceutical composition and ending 8 days, 7 days, 6 days, 5 days, 4 days, 3 days, 2 days, or 1 day prior to administration of the pharmaceutical composition. In yet other embodiments, the lymphodepletion regimen comprises administering a purine analog (e.g., pentostatin) once daily starting 8 days prior to administration of the pharmaceutical composition and ending 7 days, 6 days, 5 days, 4 days, 3 days, 2 days, or 1 day prior to administration of the pharmaceutical composition. In other embodiments, the lymphodepletion regimen comprises administering a purine analog (e.g., pentostatin) once daily starting 7 days prior to administration of the pharmaceutical composition and ending 6 days, 5 days, 4 days, 3 days, 2 days, or 1 day prior to administration of the pharmaceutical composition. In still other embodiments, the lymphodepletion regimen comprises administering a purine analog (e.g., pentostatin) once daily starting 6 days prior to administration of the pharmaceutical composition and ending 5 days, 4 days, 3 days, 2 days, or 1 day prior to administration of the pharmaceutical composition. In yet other embodiments, the lymphodepletion regimen comprises administering a purine analog (e.g., pentostatin) once daily starting 5 days prior to administration of the pharmaceutical composition and ending 4 days, 3 days, 2 days, or 1 day prior to administration of the pharmaceutical composition. In other embodiments, the lymphodepletion regimen comprises administering a purine analog (e.g., pentostatin) once daily starting 4 days prior to administration of the pharmaceutical composition and ending 3 days, 2 days, or 1 day prior to administration of the pharmaceutical composition. In still other embodiments, the lymphodepletion regimen comprises administering a purine analog (e.g., pentostatin) once daily starting 3 days prior to administration of the pharmaceutical composition and ending 2 days or 1 day prior to administration of the pharmaceutical composition. In yet other embodiments, the lymphodepletion regimen comprises administering a purine analog (e.g., pentostatin) once daily starting 2 days prior to administration of the pharmaceutical composition and ending 1 day prior to administration of the pharmaceutical composition.

In other embodiments, the lymphodepletion regimen comprises administering a purine analog (e.g., pentostatin) once daily every 4 days, every 3 days, or every 2 days. In some such embodiments, the lymphodepletion regimen comprises administering a purine analog (e.g., pentostatin) once daily every 4 days, every 3 days, or every 2 days starting 15 days, 14 days, 13 days, 12 days, 11 days, 10 days, 9 days, 8 days, 7 days, 6 days, 5 days, 4 days, 3 days, or 2 days prior to administration of the pharmaceutical composition, such that a purine analog (e.g., pentostatin) is not administered on the same day as, or following administration of, the pharmaceutical composition. In some of these embodiments, the lymphodepletion regimen comprises administering a purine analog (e.g., pentostatin) once daily every 3 days starting 15 days prior to administration of the pharmaceutical composition and ending 3 days prior to administration of the pharmaceutical composition, starting 14 days prior to administration of the pharmaceutical composition and ending 2 days prior to administration of the pharmaceutical composition, starting 13 days prior to administration of the pharmaceutical composition and ending 1 day prior to administration of the pharmaceutical composition, starting 12 days prior to administration of the pharmaceutical composition and ending 3 days prior to administration of the pharmaceutical composition, starting 11 days prior to administration of the pharmaceutical composition and ending 2 days prior to administration of the pharmaceutical composition, starting 10 days prior to administration of the pharmaceutical composition and ending 1 day prior to administration of the pharmaceutical composition, starting 9 days prior to administration of the pharmaceutical composition and ending 3 days prior to administration of the pharmaceutical composition, starting 8 days prior to administration of the pharmaceutical composition and ending 2 days prior to administration of the pharmaceutical composition, starting 7 days prior to administration of the pharmaceutical composition and ending 1 day prior to administration of the pharmaceutical composition, starting 6 days prior to administration of the pharmaceutical composition and ending 3 days prior to administration of the pharmaceutical composition, starting 5 days prior to administration of the pharmaceutical composition and ending 2 days prior to administration of the pharmaceutical composition, or starting 4 days prior to administration of the pharmaceutical composition and ending 1 day prior to administration of the pharmaceutical composition. In particular embodiments, the lymphodepletion regimen comprises administering a purine analog (e.g., pentostatin) once daily every 3 days, starting 9 days and ending 3 days prior to administration of the pharmaceutical composition.

In some embodiments, a purine analog (e.g., pentostatin) is administered intravenously.

In some of these embodiments, the lymphodepletion regimen comprises administering a purine analog (e.g., pentostatin) once daily at a dose of about 1 to about 30 mg/m²/day, about 1 to about 20 mg/m²/day, about 1 to about 10 mg/m²/day, about 2 to about 20 mg/m²/day, or about 2 to about 10 mg/m²/day, including but not limited to about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mg/m²/day every 3 days starting 15 days prior to administration of the pharmaceutical composition and ending 3 days prior to administration of the pharmaceutical composition, starting 14 days prior to administration of the pharmaceutical composition and ending 2 days prior to administration of the pharmaceutical composition, starting 13 days prior to administration of the pharmaceutical composition and ending 1 day prior to administration of the pharmaceutical composition, starting 12 days prior to administration of the pharmaceutical composition and ending 3 days prior to administration of the pharmaceutical composition, starting 11 days prior to administration of the pharmaceutical composition and ending 2 days prior to administration of the pharmaceutical composition, starting 10 days prior to administration of the pharmaceutical composition and ending 1 day prior to administration of the pharmaceutical composition, starting 9 days prior to administration of the pharmaceutical composition and ending 3 days prior to administration of the pharmaceutical composition, starting 8 days prior to administration of the pharmaceutical composition and ending 2 days prior to administration of the pharmaceutical composition, starting 7 days prior to administration of the pharmaceutical composition and ending 1 day prior to administration of the pharmaceutical composition, starting 6 days prior to administration of the pharmaceutical composition and ending 3 days prior to administration of the pharmaceutical composition, starting 5 days prior to administration of the pharmaceutical composition and ending 2 days prior to administration of the pharmaceutical composition, or starting 4 days prior to administration of the pharmaceutical composition and ending 1 day prior to administration of the pharmaceutical composition. In particular embodiments, the lymphodepletion regimen comprises administering about 4 mg/m²/day of a purine analog (e.g., pentostatin) once daily every 3 days starting 9 days and ending 3 days prior to administration of the pharmaceutical composition.

In particular embodiments, the lymphodepletion regimen comprises administering about 200 mg/day an alkylating agent (e.g., cyclophosphamide) orally once daily starting 7 days and ending 1 day prior to administration of the pharmaceutical composition, and administering about 4 mg/m²/day a purine analog (e.g., pentostatin) intravenously every 3 days starting 9 days and ending 3 days prior to administration of the pharmaceutical composition.

In some embodiments, the lymphodepletion regimen does not comprise administering an effective dose of a biological lymphodepletion agent. In some embodiments, the lymphodepletion regimen does not comprise administering a biological lymphodepletion agent. Non-limiting examples of a biological lymphodepletion agent include monoclonal antibodies or fragments thereof. Such monoclonal antibodies or fragments thereof can have specificity for a T cell antigen. In some embodiments, the monoclonal antibody or fragment thereof is an anti-CD52 monoclonal antibody or fragment thereof, or an anti-CD3 antibody or fragment thereof. In certain embodiments, the monoclonal antibody is alemtuzumab or ALLO-647. In some embodiments, the lymphodepletion regimen includes administration of a biological lymphodepletion agent in an amount no greater than 1.0 mg/kg during the 7 day period preceding administration of the pharmaceutical composition. In some embodiments, the lymphodepletion regimen includes administration of a biological lymphodepletion agent in an amount no greater than 0.75 mg/kg, 0.5 mg/kg, 0.25 mg/kg, or 0.1 mg/kg during the 7 day period preceding administration of the pharmaceutical composition. In certain embodiments, the lymphodepletion regimen includes administration of a biological lymphodepletion agent in an amount no greater than 0.1 mg/kg during the 7 day period preceding administration of the pharmaceutical composition.

In certain embodiments, the presently disclosed methods further comprise administering an effective dose of an immunosuppressant agent to the subject. In some of these embodiments, the immunosuppressant agent comprises an mTOR inhibitor (such as sirolimus, everolimus, temsirolimus, deforolimus, or ridaforolimus). In some of these embodiments, the immunosuppressant agent comprises sirolimus. The administration of sirolimus results in an effective serum concentration of sirolimus in the subject, which in some embodiments is about 15 ng/mL to about 30 ng/mL, about 20 ng/mL to about 30 ng/mL, about 15 ng/mL to about 20 ng/mL, or about 15 ng/mL to about 25 ng/mL. In some of these embodiments, the effective dose of sirolimus results in an effective serum concentration of sirolimus in the subject of about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, or about 30 ng/mL. In particular embodiments, the effective dose of sirolimus results in an effective serum concentration of sirolimus in the subject of about 20 ng/mL.

In some embodiments, sirolimus is administered once daily starting 12 days, 11 days, 10 days, 9 days, 8 days, 7 days, 6 days, 5 days, 4 days, 3 days, 2 days, or 1 day, prior to administration of the pharmaceutical composition. In particular embodiments, sirolimus is administered once daily staring 1 day prior to administration of the pharmaceutical composition. In certain embodiments, sirolimus is administered once on the day of administration of the pharmaceutical composition, and once daily each day thereafter ending 14 days, 15 days, 16 days, 17 days, 18 days, 19 days, 20 days, 21 days, 22 days, 23 days, 24 days, 25 days, 26 days, 27 days, 28 days, or longer, after administration of the pharmaceutical composition. In particular embodiments, sirolimus is administered once on the day of administration of the pharmaceutical composition and once daily each day thereafter ending 21 days after administration of the pharmaceutical composition.

In some embodiments, sirolimus is administered orally.

In some of those embodiments wherein the presently disclosed methods further comprise administering an effective dose of sirolimus, the subject is first administered one loading dose of sirolimus, followed by administration of a maintenance dose of sirolimus once daily in order to achieve the effective serum concentration of sirolimus, wherein the once daily maintenance dose of sirolimus begins the day after the loading dose of sirolimus is administered to the subject.

In some of these embodiments, the loading dose of sirolimus is about 5 mg to about 25 mg, about 5 mg to about 20 mg, about 5 mg to about 18 mg, about 5 mg to about 16 mg, about 8 mg to about 25 mg, about 8 mg to about 20 mg, about 8 mg to about 18 mg, about 8 mg to about 16 mg, about 10 mg to about 25 mg, about 10 mg to about 20 mg, about 10 mg to about 18 mg, about 10 mg to about 16 mg, about 12 mg to about 25 mg, about 12 mg to about 20 mg, about 12 mg to about 18 mg, or about 12 mg to about 16 mg. In some of these embodiments, the loading dose of sirolimus is about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg. In particular embodiments, the loading dose of sirolimus is about 16 mg. In some embodiments, the maintenance dose of sirolimus is about 1 mg to about 15 mg, about 1 mg to about 10 mg, about 1 mg to about 8 mg, about 1 mg to about 5 mg, about 2 mg to about 15 mg, about 2 mg to about 10 mg, about 2 mg to about 8, mg, about 2 mg to about 5 mg, about 3 mg to about 15 mg, about 3 mg to about 10 mg, about 3 mg to about 8 mg, or about 3 mg to about 5 mg. In some of these embodiments, the maintenance dose of sirolimus is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg. In particular embodiments, the maintenance dose of sirolimus is about 4 mg. In certain embodiments, the loading dose of sirolimus is about 5 mg to about 25 mg, about 5 mg to about 20 mg, about 8 mg to about 20 mg, about 8 mg to about 18 mg, about 10 mg to about 18 mg, or about 12 mg to about 18 mg, including but not limited to about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, and the maintenance dose of sirolimus is about 1 mg to about 15 mg, about 1 mg to about 10 mg, about 2 mg to about 8, mg, or about 3 mg to about 5 mg, including but not limited to about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg. In some of these embodiments, the loading dose of sirolimus is about 16 mg and the maintenance dose of sirolimus is about 4 mg.

In some embodiments, the loading dose of sirolimus is administered once 8 days, 7 days, 6 days, 5 days, 4 days, 3 days, 2 days, or 1 day prior to administration of the pharmaceutical composition, and the maintenance dose is administered once daily beginning one day after the loading dose is administered. In certain embodiments, following administration of the loading dose, the maintenance dose is administered once daily until 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day after administration of the pharmaceutical composition. In some of these embodiments, a loading dose is administered 1 day prior to administration of the pharmaceutical composition, and a maintenance dose of sirolimus is administered once daily beginning on the same day as administration of the pharmaceutical composition, and ending 21 days after administration of the pharmaceutical composition.

In certain embodiments, the immunosuppressant agent, such as sirolimus, can be administered orally. In particular embodiments, the loading dose of sirolimus is administered orally. In some embodiments, the maintenance dose is administered orally.

In particular embodiments, the lymphodepletion regimen comprises administering about 4 mg/m²/day of a purine analog (e.g., pentostatin) once daily every 3 days starting 9 days and ending 3 days prior to administration of the pharmaceutical composition, administering about 200 mg/day an alkylating agent (e.g., cyclophosphamide) once daily starting 7 days and ending 1 day prior to administration of the pharmaceutical composition, and administering a loading dose of sirolimus of about 16 mg once 1 day prior to administration of the pharmaceutical composition and a maintenance dose of sirolimus of about 4 mg once daily beginning on the same day as administration of the pharmaceutical composition and ending 21 days after administration of the pharmaceutical composition.

In another aspect, the invention provides a method for reducing the number of target cells in a subject, the method comprising: (a) administering to the subject an immunosuppression regimen that includes administering one or more effective doses of an mTOR inhibitor (such as sirolimus, everolimus, temsirolimus, deforolimus, or ridaforolimus); and (b) administering to the subject an effective dose of a pharmaceutical composition comprising a population of human immune cells, wherein a plurality of the human immune cells are genetically-modified human immune cells that express a CAR or an exogenous TCR, and wherein the CAR or the exogenous TCR comprises an extracellular ligand-binding domain having specificity for an antigen on the target cells.

In certain embodiments of the method, the mTOR inhibitor is sirolimus.

The administration of sirolimus results in an effective serum concentration of sirolimus in the subject, which in some embodiments is about 15 ng/mL to about 30 ng/mL, about 20 ng/mL to about 30 ng/mL, about 15 ng/mL to about 20 ng/mL, or about 15 ng/mL to about 25 ng/mL. In some of these embodiments, the effective dose of sirolimus results in an effective serum concentration of sirolimus in the subject of about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, or about 30 ng/mL. In particular embodiments, the effective dose of sirolimus results in an effective serum concentration of sirolimus in the subject of about 20 ng/mL.

In some embodiments, sirolimus is administered once daily starting 12 days, 11 days, 10 days, 9 days, 8 days, 7 days, 6 days, 5 days, 4 days, 3 days, 2 days, or 1 day, prior to administration of the pharmaceutical composition. In particular embodiments, sirolimus is administered once daily staring 1 day prior to administration of the pharmaceutical composition. In certain embodiments, sirolimus is administered once on the day of administration of the pharmaceutical composition, and once daily each day thereafter ending 14 days, 15 days, 16 days, 17 days, 18 days, 19 days, 20 days, 21 days, 22 days, 23 days, 24 days, 25 days, 26 days, 27 days, 28 days, or longer, after administration of the pharmaceutical composition. In particular embodiments, sirolimus is administered once on the day of administration of the pharmaceutical composition and once daily each day thereafter ending 21 days after administration of the pharmaceutical composition.

In some embodiments, sirolimus is administered orally.

In some embodiments, the subject is first administered one loading dose of sirolimus, followed by administration of a maintenance dose of sirolimus once daily in order to achieve the effective serum concentration of sirolimus, wherein the once daily maintenance dose of sirolimus begins the day after the loading dose of sirolimus is administered to the subject.

In some of these embodiments, the loading dose of sirolimus is about 5 mg to about 25 mg, about 5 mg to about 20 mg, about 5 mg to about 18 mg, about 5 mg to about 16 mg, about 8 mg to about 25 mg, about 8 mg to about 20 mg, about 8 mg to about 18 mg, about 8 mg to about 16 mg, about 10 mg to about 25 mg, about 10 mg to about 20 mg, about 10 mg to about 18 mg, about 10 mg to about 16 mg, about 12 mg to about 25 mg, about 12 mg to about 20 mg, about 12 mg to about 18 mg, or about 12 mg to about 16 mg. In some of these embodiments, the loading dose of sirolimus is about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg. In particular embodiments, the loading dose of sirolimus is about 16 mg. In some embodiments, the maintenance dose of sirolimus is about 1 mg to about 15 mg, about 1 mg to about 10 mg, about 1 mg to about 8 mg, about 1 mg to about 5 mg, about 2 mg to about 15 mg, about 2 mg to about 10 mg, about 2 mg to about 8, mg, about 2 mg to about 5 mg, about 3 mg to about 15 mg, about 3 mg to about 10 mg, about 3 mg to about 8 mg, or about 3 mg to about 5 mg. In some of these embodiments, the maintenance dose of sirolimus is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg. In particular embodiments, the maintenance dose of sirolimus is about 4 mg. In certain embodiments, the loading dose of sirolimus is about 5 mg to about 25 mg, about 5 mg to about 20 mg, about 8 mg to about 20 mg, about 8 mg to about 18 mg, about 10 mg to about 18 mg, or about 12 mg to about 18 mg, including but not limited to about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, and the maintenance dose of sirolimus is about 1 mg to about 15 mg, about 1 mg to about 10 mg, about 2 mg to about 8, mg, or about 3 mg to about 5 mg, including but not limited to about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg. In some of these embodiments, the loading dose of sirolimus is about 16 mg and the maintenance dose of sirolimus is about 4 mg.

In some embodiments, the loading dose of sirolimus is administered once 8 days, 7 days, 6 days, 5 days, 4 days, 3 days, 2 days, or 1 day prior to administration of the pharmaceutical composition, and the maintenance dose is administered once daily beginning one day after the loading dose is administered. In certain embodiments, following administration of the loading dose, the maintenance dose is administered once daily until 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day after administration of the pharmaceutical composition. In some of these embodiments, a loading dose is administered 1 day prior to administration of the pharmaceutical composition, and a maintenance dose of sirolimus is administered once daily beginning on the same day as administration of the pharmaceutical composition, and ending 21 days after administration of the pharmaceutical composition.

In certain embodiments, sirolimus, can be administered orally. In particular embodiments, the loading dose of sirolimus is administered orally. In some embodiments, the maintenance dose is administered orally.

In some embodiments, the method further comprises administering to the subject a lymphodepletion regimen, wherein the lymphodepletion regimen includes one or more effective doses of at least one lymphodepletion agent, and wherein the lymphodepletion regimen is administered prior to administration of the pharmaceutical composition. In some such embodiments, the at least one lymphodepletion agent includes a compound capable of inducing interstrand cross-links within DNA (such as cisplatin, mitomycin C, carmustine, psoralen or nitrogen mustard-derived alkylating agents like cyclophosphamide, ifosfamide, chlorambucil, uramustine, melphalan, and bendamustine). In certain embodiments, the at least one lymphodepletion agent includes cyclophosphamide. In some such embodiments, the at least one lymphodepletion agent includes a purine analog. In some such embodiments, the at least one lymphodepletion agent includes pentostatin. In certain embodiments, the lymphodepletion regimen includes one or more effective doses of cyclophosphamide and one or more effective doses of pentostatin, such as the doses of each described herein. In some embodiments, the at least one lymphodepletion agent includes fludarabine. In certain embodiments, the lymphodepletion regimen includes one or more effective doses of cyclophosphamide and one or more effective doses of fludarabine.

In some embodiments, the lymphodepletion regimen does not comprise administering an effective dose of a biological lymphodepletion agent. In some embodiments, the lymphodepletion regimen does not comprise administering a biological lymphodepletion agent. Non-limiting examples of a biological lymphodepletion agent include monoclonal antibodies or fragments thereof. Such monoclonal antibodies or fragments thereof can have specificity for a T cell antigen. In some embodiments, the monoclonal antibody or fragment thereof is an anti-CD52 monoclonal antibody or fragment thereof, or an anti-CD3 antibody or fragment thereof. In certain embodiments, the monoclonal antibody is alemtuzumab or ALLO-647. In some embodiments, the lymphodepletion regimen includes administration of a biological lymphodepletion agent in an amount no greater than 1.0 mg/kg during the 7 day period preceding administration of the pharmaceutical composition. In some embodiments, the lymphodepletion regimen includes administration of a biological lymphodepletion agent in an amount no greater than 0.75 mg/kg, 0.5 mg/kg, 0.25 mg/kg, or 0.1 mg/kg during the 7 day period preceding administration of the pharmaceutical composition. In certain embodiments, the lymphodepletion regimen includes administration of a biological lymphodepletion agent in an amount no greater than 0.1 mg/kg during the 7 day period preceding administration of the pharmaceutical composition. In some embodiments, the lymphodepletion regimen includes no more than a minimal effective dose of a biological lymphodepletion agent.

In some embodiments of the methods described herein, the pharmaceutical composition is administered at a dose of between about 1×10⁴ and about 1×10⁸, about 1×10⁴ and about 3×10⁶, about 1×10⁵ and about 1×10⁷, about 1×10⁵ and about 6×10⁶, about 1×10⁵ and about 3×10⁶, about 3×10⁵ and about 6×10⁶, and about 3×10⁵ and about 3×10⁶ genetically-modified human immune cells/kg. In some of these embodiments, the pharmaceutical composition is administered at a dose of about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ genetically-modified human immune cells/kg. In some of these embodiments, the pharmaceutical composition is administered at a dose of about 1×10⁶ genetically-modified human immune cells/kg. In some of these embodiments, the pharmaceutical composition is administered at a dose of about 2×10⁶ genetically-modified human immune cells/kg. In some of these embodiments, the pharmaceutical composition is administered at a dose of about 3×10⁶ genetically-modified human immune cells/kg. In some of these embodiments, the pharmaceutical composition is administered at a dose of about 4×10⁶ genetically-modified human immune cells/kg. In some of these embodiments, the pharmaceutical composition is administered at a dose of about 5×10⁶ genetically-modified human immune cells/kg. In some of these embodiments, the pharmaceutical composition is administered at a dose of about 6×10⁶ genetically-modified human immune cells/kg. In some of these embodiments, the pharmaceutical composition is administered at a dose of about 7×10⁶ genetically-modified human immune cells/kg. In some of these embodiments, the pharmaceutical composition is administered at a dose of about 8×10⁶ genetically-modified human immune cells/kg. In some of these embodiments, the pharmaceutical composition is administered at a dose of about 9×10⁶ genetically-modified human immune cells/kg. In some of these embodiments, the pharmaceutical composition is administered at a dose of about 1×10⁷ genetically-modified human immune cells/kg. In particular embodiments, the effective dose of the pharmaceutical composition comprises no more than 3×10⁸ genetically-modified human immune cells.

In some embodiments of the method described herein, the methods comprise administering another dose of the pharmaceutical composition to the subject.

In some embodiments of the methods described herein, the genetically-modified human immune cells are human T cells, or cells derived therefrom. In some embodiments of the methods described herein, the genetically-modified human immune cells are human natural killer (NK) cells, or cells derived therefrom. In certain embodiments, the genetically-modified human immune cells are derived from induced pluripotent stem cells (iPSCs) that have been differentiated into the genetically-modified human immune cells described herein.

In some embodiments of the methods described herein, the target cells that are being reduced with the presently disclosed methods are cancer cells. In some of these embodiments, the cancer cells are from a cancer of B cell origin or multiple myeloma. The cancer of B cell origin can be acute lymphoblastic leukemia (ALL), chronic lymphocytic leukemia (CLL), small lymphocytic lymphoma (SLL), or non-Hodgkin lymphoma (NHL). In some of those embodiments wherein the target cells are NHL cells, the NHL is mantle cell lymphoma (MCL) or diffuse large B cell lymphoma (DLBCL).

In certain embodiments of the methods described herein, the subject in which target cells are being reduced using the presently disclosed methods is refractory to prior immunotherapy (e.g., CAR T or CAR NK immunotherapy).

In particular embodiments of the methods described herein, the genetically-modified human immune cells comprise an inactivated TCR alpha gene or an inactivated TCR beta gene. In some of these embodiments, a transgene encoding the CAR or the exogenous TCR is inserted into the genome of the genetically-modified human immune cells within the TCR alpha gene or the TCR beta gene, wherein the transgene disrupts expression of the TCR alpha gene or the TCR beta gene. In certain embodiments, the transgene encoding the CAR or the exogenous TCR is inserted into a TCR alpha constant region gene. In some of these embodiments, the transgene encoding the CAR or the exogenous TCR is inserted into an engineered meganuclease recognition sequence comprising SEQ ID NO: 1 within the TCR alpha constant region gene. In some of these embodiments, the transgene encoding the CAR or the exogenous TCR is inserted between positions 13 and 14 of SEQ ID NO: 1 within the TCR alpha constant region gene.

In some embodiments of the methods described herein, the genetically-modified human immune cells do not have detectable cell surface expression of an endogenous alpha/beta TCR. In certain embodiments, the genetically-modified human immune cells do not have detectable cell surface expression of CD3.

In some embodiments of the methods described herein, the extracellular ligand-binding domain of the CAR or the exogenous TCR of the genetically-modified human immune cells has specificity for CD19, CD20, or B cell maturation antigen (BCMA).

In certain embodiments of the methods described herein, extracellular ligand-binding domain of the CAR or the exogenous TCR of the genetically-modified human immune cells comprises a single-chain variable fragment (scFv). In some embodiments, the extracellular ligand-binding domain of the CAR or the exogenous TCR comprises a single-chain variable fragment (scFv) comprising a heavy chain variable domain (VH) of SEQ ID NO: 3 and a light chain variable domain (VL) of SEQ ID NO: 4, or a VH of SEQ ID NO: 6 and a VL of SEQ ID NO: 7. In certain embodiments, the CAR comprises a CD8 alpha hinge domain. In some embodiments, the CAR comprises a CD8 alpha transmembrane domain. In certain embodiments, the CAR comprises a co-stimulatory domain comprising one or more TRAF-binding domains. In particular embodiments, the CAR comprises a co-stimulatory domain comprising a first domain comprising SEQ ID NO: 9 and a second domain comprising SEQ ID NO: 10 or 11. In other embodiments, the CAR comprises a novel 6 (N6) co-stimulatory domain or a 4-1BB co-stimulatory domain. In certain embodiments, the CAR comprises CD3 zeta intracellular signaling domain. In particular embodiments, the CAR comprises an amino acid sequence having at least 80% sequence identity to SEQ ID NO: 5 and has specificity for CD19, or an amino acid sequence having at least 80% sequence identity to SEQ ID NO: 8 and has specificity for CD20. In some of these embodiments, the CAR comprises an amino acid sequence of SEQ ID NO: 5 or 8.

In some embodiments of the methods described herein, the genetically-modified human immune cells represent between about 40% and about 75%, about 50% and about 70%, and about 55% and about 65%, including but not limited to about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 635%, about 64%, about 65%, about 66%, about 67%, about 68%, about 69%, and about 70% of the human immune cells in the pharmaceutical composition. In some of these embodiments, the genetically-modified human immune cells represent between about 50% and about 70% of the human immune cells in the pharmaceutical composition.

In certain embodiments of the methods described herein, the genetically-modified human immune cells proliferate in vivo for at least one day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, or longer, following administration of the pharmaceutical composition. In some of these embodiments, the genetically-modified human immune cells proliferate in vivo between about day 1 and about day 28, about day 1 and about day 25, about day 1 and about day 23, about day 1 and about day 21, about day 1 and about day 19, about day 1 and about day 17, about day 1 and about day 15, or about day 1 and about day 10, following administration of the pharmaceutical composition. In some of these embodiments, the genetically-modified human immune cells of the pharmaceutical composition proliferate in vivo between about day 1 and about day 21.

In some embodiments of the methods described herein, the number of copies of the CAR or the exogenous TCR transgene per μg of DNA in peripheral blood mononuclear cells is elevated for up to 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer after administration of the pharmaceutical composition when compared to the number of copies present prior to administration. In some of these embodiments, the number of copies of the CAR or the exogenous TCR transgene per μg of DNA in peripheral blood mononuclear cells is elevated for up to 21 days after administration of the pharmaceutical composition when compared to the number of copies present prior to administration.

In certain embodiments of the methods described herein, the serum concentration of C-reactive protein, ferritin, IL-6, interferon gamma, or any combination thereof, is elevated compared to the concentration at day 0 for at least 1 day, 2 days, 3 days, 4 days, 5 days, 6 days, 7 days, 8 days, 9 days, 10 days, 15 days, 20 days, 25 days, or longer, following administration of the pharmaceutical composition.

In some embodiments of the methods described herein, the method is an immunotherapy for the treatment of a disease, such as cancer, and the subject achieves a partial response or a complete response to the method of immunotherapy. In some of these embodiments, the partial response or the complete response is maintained through at least 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, or longer after administration of the pharmaceutical composition. In some of these embodiments, the partial response or the complete response is maintained through at least 28 days after administration of the pharmaceutical composition.

In some embodiments of the methods described herein, the target cells are ALL, MCL, or DLBCL cells, and the lymphodepletion regimen comprises administering: a) an alkylating agent (e.g., cyclophosphamide) at a dose of about 200 mg/day to the subject once daily starting 7 days and ending 1 day prior to administration of the pharmaceutical composition; and b) a purine analog (e.g., pentostatin) at a dose of about 4 mg/m²/day to the subject once every 3 days starting 9 days and ending 3 days prior to administration of the pharmaceutical composition; wherein the genetically-modified human immune cells are CAR T cells, wherein the pharmaceutical composition is administered at a dose of between about 3×10⁴ and 3×10⁶ CAR T cells/kg and not to exceed 3×10⁸ CAR T cells, wherein a transgene encoding the CAR is inserted into a TCR alpha constant region gene, and wherein the CAR comprises an scFv having specificity for CD19, a CD8 alpha hinge domain, a CD8 alpha transmembrane domain, a co-stimulatory domain comprising one or more TRAF-binding domains, and a CD3 zeta intracellular signaling domain.

In some embodiments of the methods described herein, the target cells are ALL, MCL, or DLBCL cells, wherein the lymphodepletion regimen comprises administering: a) an alkylating agent (e.g., cyclophosphamide) at a dose of about 200 mg/day to the subject once daily starting 7 days and ending 1 day prior to administration of the pharmaceutical composition; and b) a purine analog (e.g., pentostatin) at a dose of about 4 mg/m²/day to the subject once every 3 days starting 9 days and ending 3 days prior to administration of the pharmaceutical composition; wherein the genetically-modified human immune cells are CAR T cells, wherein the pharmaceutical composition is administered at a dose of between about 3×10⁴ and 3×10⁶ CAR T cells/kg and not to exceed 3×10⁸ CAR T cells, wherein a transgene encoding the CAR is inserted into a TCR alpha constant region gene, and wherein the CAR comprises an scFv comprising a VH domain of SEQ ID NO: 3 and a VL domain of SEQ ID NO: 4, a CD8 alpha hinge domain, a CD8 alpha transmembrane domain, an N6 co-stimulatory domain, and a CD3 zeta intracellular signaling domain.

In some embodiments of the methods described herein, the target cells are NHL, CLL, or SLL cells, wherein the lymphodepletion regimen comprises administering: a) an alkylating agent (e.g., cyclophosphamide) at a dose of about 200 mg/day to the subject once daily starting 7 days and ending 1 day prior to administration of the pharmaceutical composition; and b) a purine analog (e.g., pentostatin) at a dose of about 4 mg/m²/day to the subject once every 3 days starting 9 days and ending 3 days prior to administration of the pharmaceutical composition; wherein the genetically-modified human immune cells are CAR T cells, wherein the pharmaceutical composition is administered at a dose of between about 3×10⁴ and 3×10⁶ CAR T cells/kg and not to exceed 3×10⁸ CAR T cells, wherein a transgene encoding the CAR is inserted into a TCR alpha constant region gene, and wherein the CAR comprises an scFv having specificity for CD20, a CD8 alpha hinge domain, a CD8 alpha transmembrane domain, a co-stimulatory domain comprising one or more TRAF-binding domains, and a CD3 zeta intracellular signaling domain.

In some embodiments of the methods described herein the target cells are NHL, CLL, or SLL cells, wherein the lymphodepletion regimen comprises administering: a) an alkylating agent (e.g., cyclophosphamide) at a dose of about 200 mg/day to the subject once daily starting 7 days and ending 1 day prior to administration of the pharmaceutical composition; and b) a purine analog (e.g., pentostatin) at a dose of about 4 mg/m²/day to the subject once every 3 days starting 9 days and ending 3 days prior to administration of the pharmaceutical composition; wherein the genetically-modified human immune cells are CAR T cells, wherein the pharmaceutical composition is administered at a dose of between about 3×10⁴ and 3×10⁶ CAR T cells/kg and not to exceed 3×10⁸ CAR T cells, wherein a transgene encoding the CAR is inserted into a TCR alpha constant region gene, and wherein the CAR comprises an scFv comprising a VH domain of SEQ ID NO: 6 and a VL domain of SEQ ID NO: 7, a CD8 alpha hinge domain, a CD8 alpha transmembrane domain, an N6 co-stimulatory domain, and a CD3 zeta intracellular signaling domain.

In some embodiments of the methods described herein, the target cells are multiple myeloma cells, wherein the lymphodepletion regimen comprises administering: a) an alkylating agent (e.g., cyclophosphamide) at a dose of about 200 mg/day to the subject once daily starting 7 days and ending 1 day prior to administration of the pharmaceutical composition; and b) a purine analog (e.g., pentostatin) at a dose of about 4 mg/m²/day to the subject once every 3 days starting 9 days and ending 3 days prior to administration of the pharmaceutical composition; wherein the genetically-modified human immune cells are CAR T cells, wherein the pharmaceutical composition is administered at a dose of between about 3×10⁴ and 3×10⁶ CAR T cells/kg and not to exceed 3×10⁸ CAR T cells, wherein a transgene encoding the CAR is inserted into a TCR alpha constant region gene, and wherein the CAR comprises an scFv having specificity for BCMA, a CD8 alpha hinge domain, a CD8 alpha transmembrane domain, a co-stimulatory domain comprising one or more TRAF-binding domains, and a CD3 zeta intracellular signaling domain.

In some embodiments of the methods described herein, the target cells are multiple myeloma cells, wherein the lymphodepletion regimen comprises administering: a) an alkylating agent (e.g., cyclophosphamide) at a dose of about 200 mg/day to the subject once daily starting 7 days and ending 1 day prior to administration of the pharmaceutical composition; and b) a purine analog (e.g., pentostatin) at a dose of about 4 mg/m²/day to the subject once every 3 days starting 9 days and ending 3 days prior to administration of the pharmaceutical composition; wherein the genetically-modified human immune cells are CAR T cells, wherein the pharmaceutical composition is administered at a dose of between about 3×10⁴ and 3×10⁶ CAR T cells/kg and not to exceed 3×10⁸ CAR T cells, wherein a transgene encoding the CAR is inserted into a TCR alpha constant region gene, and wherein the CAR comprises an scFv comprising a VH domain and a VL domain of a BCMA-specific monoclonal antibody, a CD8 alpha hinge domain, a CD8 alpha transmembrane domain, an N6 co-stimulatory domain, and a CD3 zeta intracellular signaling domain.

In some embodiments of the methods described herein, the method further comprises administering an effective dose of sirolimus to the subject that results in an effective serum concentration of sirolimus in the subject of about 20 ng/mL, wherein one loading dose of about 16 mg of sirolimus is administered one day prior to administration of the pharmaceutical composition and a maintenance dose of about 4 mg of sirolimus is administered once daily starting on the day the pharmaceutical composition is administered and ending 21 days after administration of the pharmaceutical composition.

In some embodiments of the methods described herein, the methods further comprise manufacturing the genetically-modified human immune cells. Manufacturing comprises a first culturing step wherein isolated human immune cells (e.g., human T cell or NK cells) are cultured in media for 3 days with anti-CD3 and anti-CD28 antibodies bound to a matrix or particle, followed by electroporation of the isolated human immune cells to introduce mRNA encoding an engineered nuclease having specificity for a recognition sequence within the TCR alpha gene, wherein the engineered nuclease is expressed in the human immune cells and generates a cleavage site at the recognition sequence. Next, the isolated human immune cells are transduced with a recombinant AAV vector comprising a donor template, wherein the donor template comprises a transgene encoding the CAR or the exogenous TCR, and wherein the donor template is flanked by a 5′ homology arm having homology to sequences 5′ upstream of the cleavage site, and by a 3′ homology arm having homology to sequences 3′ downstream of the cleavage site, wherein the donor template is inserted into the genome of the isolated human immune cells at the cleavage site, followed by a second culturing step wherein the isolated human immune cells are cultured in media for about 5 days. Finally, the isolated human immune cells that express cell surface CD3 are removed using anti-CD3 antibodies, and a third culturing step is performed wherein the isolated human immune cells are cultured in media to generate the plurality of genetically-modified human immune cells used in the pharmaceutical composition. In some of these embodiments, manufacturing is completed in about 10 days or less. In certain embodiments, the engineered nuclease is an engineered meganuclease, a zinc finger nuclease, a TALEN, a compact TALEN, a CRISPR system nuclease, or a megaTAL. In particular embodiments, the engineered nuclease is an engineered meganuclease. In some of these embodiments, the engineered meganuclease has specificity for a recognition sequence comprising SEQ ID NO: 1. The engineered meganuclease can comprise an amino acid sequence of SEQ ID NO: 17 or an active variant thereof such as one having at least 80%, 85%, 90%, 91%, 92%, 93%, 94%, 95%, 96%, 97%, 98%, 99% or more sequence identity to SEQ D NO: 17.

In another aspect, the present invention provides a purine analog (e.g., pentostatin) for the reduction of target cells in a subject, wherein the reduction of target cells in a subject comprises the steps of: (a) administering to the subject a lymphodepletion regimen that comprises one or more effective doses of a purine analog (e.g., pentostatin) and one or more effective doses of an alkylating agent (e.g., cyclophosphamide); and (b) administering to the subject an effective dose of a pharmaceutical composition comprising a population of human immune cells, wherein a plurality of the human immune cells are genetically-modified human immune cells that express a chimeric antigen receptor (CAR) or an exogenous T cell receptor (TCR), wherein the lymphodepletion regimen is administered prior to administration of the pharmaceutical composition, and wherein the CAR or the exogenous TCR comprises an extracellular ligand-binding domain having specificity for an antigen on the target cells.

In another aspect, the present invention provides an alkylating agent (e.g., cyclophosphamide) for the reduction of target cells in a subject, wherein the reduction of target cells in a subject comprises the steps of: (a) administering to the subject a lymphodepletion regimen that comprises one or more effective doses of a purine analog (e.g., pentostatin) and one or more effective doses of an alkylating agent (e.g., cyclophosphamide); and (b) administering to the subject an effective dose of a pharmaceutical composition comprising a population of human immune cells, wherein a plurality of the human immune cells are genetically-modified human immune cells that express a chimeric antigen receptor (CAR) or an exogenous T cell receptor (TCR), wherein the lymphodepletion regimen is administered prior to administration of the pharmaceutical composition, and wherein the CAR or the exogenous TCR comprises an extracellular ligand-binding domain having specificity for an antigen on the target cells.

In another aspect, the present invention provides sirolimus for the reduction of target cells in a subject, wherein the reduction of target cells in a subject comprises the steps of: (a) administering to the subject an immunosuppression regimen that comprises one or more effective doses of sirolimus; and (b) administering to the subject an effective dose of a pharmaceutical composition comprising a population of human immune cells, wherein a plurality of the human immune cells are genetically-modified human immune cells that express a CAR or an exogenous TCR, and wherein the CAR or the exogenous TCR comprises an extracellular ligand-binding domain having specificity for an antigen on the target cells.

### BRIEF DESCRIPTION OF THE SEQUENCES

SEQ ID NO: 1 sets forth the nucleic acid sequence of the TRC 1-2 recognition sequence (sense).

SEQ ID NO: 2 sets forth the nucleic acid sequence of the TRC 1-2 recognition sequence (antisense).

SEQ ID NO: 3 sets forth the amino acid sequence of the heavy chain variable region of a murine anti-CD19 antibody.

SEQ ID NO: 4 sets forth the amino acid sequence of the light chain variable region of a murine anti-CD19 antibody.

SEQ ID NO: 5 sets forth the amino acid sequence of an anti-CD19 chimeric antigen receptor.

SEQ ID NO: 6 sets forth the amino acid sequence of the heavy chain variable region of a murine anti-CD20 antibody.

SEQ ID NO: 7 sets forth the amino acid sequence of the light chain variable region of a murine anti-CD20 antibody.

SEQ ID NO: 8 sets forth the amino acid sequence of an anti-CD20 chimeric antigen receptor.

SEQ ID NO: 9 sets forth the amino acid sequence of a domain found in a TRAF-binding co-stimulatory domain.

SEQ ID NO: 10 sets forth the amino acid sequence of a domain found in a TRAF-binding co-stimulatory domain.

SEQ ID NO: 11 sets forth the amino acid sequence of a domain found in a TRAF-binding co-stimulatory domain.

SEQ ID NO: 12 sets forth the amino acid sequence of a Novel 6 (N6) co-stimulatory domain.

SEQ ID NO: 13 sets forth the amino acid sequence of a 4-1BB co-stimulatory domain.

SEQ ID NO: 14 sets forth the amino acid sequence of a CD8 alpha hinge domain.

SEQ ID NO: 15 sets forth the amino acid sequence of a CD8 transmembrane domain.

SEQ ID NO: 16 sets forth the amino acid sequence of a CD3 zeta intracellular signaling domain.

SEQ ID NO: 17 sets forth the amino acid sequence of the TRC 1-2L.1592 meganuclease.

SEQ ID NO: 18 sets forth the amino acid sequence of the wild-type I-CreI meganuclease.

SEQ ID NO: 19 sets forth the nucleic acid sequence of a human T cell receptor alpha constant region gene.

SEQ ID NO: 20 sets forth the amino acid sequence of a polypeptide encoded by a human T cell receptor alpha constant region gene.

### BRIEF DESCRIPTION OF THE FIGURES

FIG. 1. Graphs with the number of K6366 CD3⁺ effector (dashed lines) or K6366 CD3⁻ CAR T targets (solid lines) plotted on the y-axes with respect to time (days) on the x-axes. A) Cultures were assembled using E:T ratio of 1:1 in the absence of rapamycin. B) Cultures were assembled using E:T ratio of 1:1 in the presence of 30 ng/ml rapamycin. C) Cultures were assembled using E:T ratio of 1:3 in the absence of rapamycin. D) Cultures were assembled using E:T ratio of 1:3 in the presence of 30 ng/ml rapamycin.

FIG. 2. Graphs with the number of HC9189 CD3⁺ effector (dashed lines) or K799 CD3⁻ CAR T targets (solid lines) plotted on the y-axes with respect to time (days) on the x-axes. A) Cultures were assembled using a 1:1 E:T ratio in the absence of rapamycin. B) Cultures were assembles using a 1:1 E:T ratio in the presence of 30 ng/ml rapamycin.

FIG. 3. Graphs with the number of OKBI4789 CD3⁺ effector (dashed lines) or K6366 CD3⁻ CAR T targets (solid lines) plotted on the y-axes with respect to time (days) on the x-axes. A) Cultures were assembled using E:T ratio of 1:1 in the absence of rapamycin. B) Cultures were assembled using E:T ratio of 1:1 in the presence of 30 ng/ml rapamycin. C) Cultures were assembled using E:T ratio of 1:3 in the absence of rapamycin. D) Cultures were assembled using E:T ratio of 1:3 in the presence of 30 ng/ml rapamycin.

FIG. 4. Graphs with the number of HC9189 CD3⁺ effector (dashed lines) or K6366 CD3⁻ CAR T targets (solid lines) plotted on the y-axes with respect to time (days) on the x-axes. A) Cultures were assembled using E:T ratio of 1:1 in the absence of rapamycin. B) Cultures were assembled using E:T ratio of 1:1 in the presence of 30 ng/ml rapamycin. C) Cultures were assembled using E:T ratio of 1:3 in the absence of rapamycin. D) Cultures were assembled using E:T ratio of 1:3 in the presence of 30 ng/ml rapamycin.

FIG. 5. Graphs showing the number of CAR T cells (y axes) plotted for each E:T ratio (indicated by x-axis labels). Horizontal dashed lines indicate the number of input CAR T cells. Shaded bars: no drug, white bars: 30 ng/ml rapamycin. A) Cell numbers with K19 stimulation are shown following 3 days. B) Cell numbers with Raji stimulation are shown following 3 days. C) Cell numbers with K19 stimulation are shown following 3 days. D) Cell numbers with Raji stimulation are shown following 6 days.

FIG. 6. Graphs showing the killing of K19 targets and Raji targets displayed as % cytolysis (y-axes) for d3 and d6 time points. Shaded bars: no drug. White bars: 30 ng/ml rapamycin. A) K19 targets on day 3. B) Raji targets on day 3. C) K19 targets on day 6. D) Raji targets on day 6.

FIG. 7. Graphs showing the number of K19 and Raji cells 6d after plating. Shaded bars: no drug. White bars: 30 ng/ml rapamycin. A) K19 targets on day 6. B) Raji targets on day 6.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.

As used herein, the term “lymphodepletion” or “lymphodepletion regimen” refers to the administration to a subject of one or more agents (e.g., lymphodepletion agents) capable of reducing endogenous lymphocytes in the subject for immunotherapy; e.g., a reduction of one or more lymphocytes (e.g., B cells, T cells, and/or NK cells) by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject undergoing treatment, relative to a pre-determined threshold, or relative to an untreated subject).

As used herein, the term “biological lymphodepletion agent” refers to a biological material, such an antibody, antibody fragment, antibody conjugate, or the like, that can be administered as part of a lymphodepletion regimen to reduce endogenous lymphocytes in the subject for immunotherapy. In some cases, such biological lymphodepletion agents can have specificity for antigens present on lymphocytes; e.g., CD52 or CD3.

As used herein, the term “chemotherapeutic lymphodepletion agents” refers to non-biological materials, such as small molecules, that can be administered as part of a lymphodepletion regimen to reduce endogenous lymphocytes in the subject for immunotherapy. In some examples, the chemotherapeutic lymphodepleting agent can be lymphodepleting but non-myeloablative.

As used herein, the term “immunosuppression” or “immunosuppression regimen” refers to the administration to a subject of one or more agents (e.g., immunosuppressant agents) capable of preventing or inhibiting activity of the subject's immune system. Immunosuppression can be through the inhibition of a cell-mediated immune response, a humoral immune response, or a combination thereof. In some embodiments, the immunosuppressant agent(s) used in the immunosuppression regimen can be anti-proliferative agents that inhibit the proliferation of immune cells, cytotoxic agents that kill immune cells, or agents that inhibit the activation of immune cells (e.g., T cells and B cells). Non-limiting examples of immunosuppressant agents include calcineurin inhibitors (e.g., cyclosporine, tacrolimus), corticosteroids (e.g., methylprednisolone, dexamethasone, prednisolone), cytotoxic immunosuppressants (e.g., azathioprine, chlorambucil, cyclophosphamide, mercaptopurine, methotrexate), immunosuppressant antibodies (e.g., antithymocyte globulins, basiliximab, infliximab), nucleoside analogs (e.g., cytarabine, fludarabine, gemcitabine, decitabine), and mTOR inhibitors (e.g., everolimus, sirolimus, temsirolimus, deforolimus, ridaforolimus). In particular embodiments of the presently disclosed methods, the immunosuppression regimen does not comprise administering a cytotoxic agent. In some embodiments, the immunosuppression regimen results in a reduction of an immune response (e.g., lymphocyte activation, cytokine levels, immune cell number) by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject undergoing treatment, relative to a pre-determined threshold, or relative to an untreated subject).

As used herein, the term “effective dose”, “effective amount”, “therapeutically effective dose”, or “therapeutically effective amount,” as used herein, refers to an amount sufficient to effect beneficial or desirable biological and/or clinical results. In some cases, an effective dose of a lymphodepletion agent is sufficient to reduce endogenous lymphocytes in the subject; e.g., a reduction of one or more lymphocytes (e.g., B cells, T cells, and/or NK cells) by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject undergoing treatment of a disease, condition or disorder, relative to a pre-determined threshold, or relative to an untreated subject). In some embodiments, the effective dose is equivalent to the suggested, recommended or allowed dose (for adults or children) provided in the drug product labeling for a lymphodepletion agent. In other cases, an effective dose of an immunosuppressant agent is sufficient to reduce an immune response in the subject; e.g., a reduction in number of one or more immune cell types, activation of one or more lymphocyte type, or levels of one or more cytokines by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject, relative to a pre-determined threshold, or relative to an untreated subject). In some embodiments, the effective dose is equivalent to the suggested, recommended or allowed dose (for adults or children) provided in the drug product labeling for an immunosuppressant agent. In other cases, an effective dose of a pharmaceutical composition comprising a population of human immune cells, wherein the population comprises a plurality of genetically-modified human immune cells, and wherein the genetically-modified human immune cells express a CAR or an exogenous TCR comprising an extracellular ligand-binding domain having specificity for an antigen on target cells, when administered following a lymphodepletion and/or immunosuppression regimen, is sufficient to reduce the target cells by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject, relative to a pre-determined threshold, or relative to an untreated subject). In some embodiments, the effective dose is equivalent to the suggested, recommended or allowed dose (for adults or children) provided in the drug product labeling for genetically-modified immune cells.

In those embodiments wherein a lymphodepletion regimen or immunosuppression regimen or tolerance regimen comprises the administration of more than one lymphodepletion, immunosuppression, or tolerance agent, the effective dose of each agent may be less than in those embodiments wherein the regimen comprises the administration of a single agent due to additive or even synergistic effects when two or more agents are used.

As used herein, the term “minimal effective dose” refers to an amount of a pharmaceutical agent that is equivalent to about 10% of the maximum allowed dose as set forth by the FDA or an equivalent government agency, for instance, in the labeling for any drug product(s) that comprise the agent.

As used herein, the term “effective serum concentration” refers to an amount of a beneficial or therapeutic agent within a subject's serum that is sufficient to effect beneficial or desirable biological and/or clinical results. The effective serum concentration is generally a range of concentrations within which the agent exerts its beneficial or therapeutic effects (i.e., its therapeutic window). The effective serum concentration can refer to the amount of the agent within the subject's serum as measured within an allotted time following administration of the agent, for example, within about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, or more. Alternatively, the effective serum concentration can be maintained at a steady state for a period of time through a dosing regimen that balances the bioavailability, absorption, distribution, metabolism and elimination of the particular agent within the subject (e.g., a constant infusion or repetitive dosing at a regular dosing interval) wherein the dose rate and clearance rate of the agent are equal. The effective serum concentration can be initially achieved with a loading dose, followed by the administration of a maintenance dose of the beneficial or therapeutic agent. Such a dosing regimen can be determined by one of ordinary skill in the art.

As used herein, the term “loading dose” refers to an amount of a beneficial or therapeutic agent that is administered to a subject that allows for the effective concentration of the agent to be reached with a single dose. Generally, the loading dose is followed by a lower maintenance dose that serves to maintain the effective concentration of the beneficial or therapeutic agent. The loading dose can be calculated as: LD=CpV_{D}/F, wherein LD is the loading dose, Cₚ is the desired peak concentration of the beneficial or therapeutic agent, VD is the volume of distribution, and F is the bioavailability of the beneficial or therapeutic agent.

As used herein, the term “maintenance dose” refers to the dose of a beneficial or therapeutic agent administered throughout a dosing regimen to maintain effective drug concentrations within a subject (e.g., an effective serum concentration). Generally, the maintenance dose is the rate of drug administration equal to the rate of elimination at steady state and can be measured using the following formula: MD=CpCL/F, wherein MD is the maintenance dose, Cₚ is the desired peak concentration of the beneficial or therapeutic agent, CL is the clearance of the beneficial or therapeutic agent in the body, and F is the bioavailability of the beneficial or therapeutic agent.

As used herein, the term “treatment”, “treating”, or “treating a subject” refers to the administration of a pharmaceutical composition disclosed herein, comprising a population of human immune cells (e.g., T cells or NK cells), to a subject having a disease, disorder or condition. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, a partial or complete reduction in the number of cancer cells present in the subject, and remission or improved prognosis. In some aspects, treatment includes the administration of a lymphodepletion regimen to reduce endogenous lymphocytes in the subject for immunotherapy.

As used herein, “target cells” refers to cells that are desired to be reduced in number using the presently disclosed methods. According to the presently disclosed methods, the target cells express an antigen that can be targeted with genetically-modified human immune cells comprising a chimeric antigen receptor (CAR) or exogenous T cell receptor (TCR), wherein the CAR or exogenous TCR comprises an extracellular ligand-binding domain having specificity for the antigen. In some embodiments, the antigen that is targeted with genetically-modified immune cells according to the presently disclosed methods is on the surface of the target cells. The target cells can be viral, bacterial, fungal, or human cells. The target cells can be disease-causing cells or cells associated with a particular disease state (e.g., autoimmune disease, cancer) or infection, such as cells infected with a virus, bacteria, fungus, or parasite. In some embodiments, the target cells are cancer cells. The target cells can be reduced using the presently disclosed methods. In some embodiments, the methods result in a reduction in the number of the target cells within the subject when compared to a control (e.g., relative to a starting amount in the subject prior to treatment according to the presently disclosed methods, relative to a pre-determined threshold, or relative to an untreated subject). That is, the number of target cells in the subject may be reduced by a percentage using the methods described herein. Such a reduction may be up to 5%, 10%, 20%, 30%, 40%, 50%, 60%, 70%, 80%, 90%, 95%, 96%, 97%, 98%, 99%, or up to 100%.

As used herein, the term “immune cell” refers to any cell that is part of the immune system (innate and/or adaptive) and is of hematopoietic origin. Non-limiting examples of immune cells include lymphocytes, B cells, T cells, monocytes, macrophages, dendritic cells, granulocytes, megakaryocytes, monocytes, macrophages, natural killer cells, myeloid-derived suppressor cells, innate lymphoid cells, platelets, red blood cells, thymocytes, leukocytes, neutrophils, mast cells, eosinophils, basophils, and granulocytes.

As used herein, the terms “T cell” and “T lymphocyte” are used interchangeably herein and refer to a white blood cell of the lymphocyte subtype that expresses T cell receptors on the cell membrane. T cells develop in the thymus gland and include both CD8+ T cells and CD4+ T cells, as well as natural killer T cells, memory T cells, gamma delta T cells, and any other lymphocytic cell that expresses a T cell receptor.

As used herein, the terms “human natural killer cell” or “human NK cell” or “natural killer cell” or “NK cell” refers to a type of cytotoxic lymphocyte critical to the innate immune system. The role NK cells play is analogous to that of cytotoxic T cells in the vertebrate adaptive immune response. NK cells provide rapid responses to virally infected cells and respond to tumor formation, acting at around 3 days after infection. Human NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely. In some cases, the human NK cell is a differentiated induced pluripotent stem cell (iPSC); e.g., an iPSC derived from a human somatic cell.

As used herein, the term “human T cell” or “isolated human T cell” refers to a T cell isolated from a human donor. In some cases, the human donor is not the subject treated according to the method (i.e., the T cells are allogeneic), but instead a healthy human donor. In some cases, the human donor is the subject treated according to the method. T cells, and cells derived therefrom, can include, for example, isolated T cells that have not been passaged in culture, or T cells that have been passaged and maintained under cell culture conditions without immortalization. In some cases, the human T cell is a differentiated induced pluripotent stem cell (iPSC); e.g., an iPSC derived from a human somatic cell.

As used herein, the term “T cell receptor alpha gene” or “TCR alpha gene” refer to the locus in a T cell which encodes the T cell receptor alpha subunit. The T cell receptor alpha gene can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” or “TRAC” refers to a coding sequence of the T cell receptor alpha gene. The TCR alpha constant region includes the wild-type sequence, and functional variants thereof, identified by NCBI Gene ID NO. 28755. See also SEQ ID NO: 19.

As used herein, the term “T cell receptor beta gene” or “TCR beta gene” refers to the locus in a T cell which encodes the T cell receptor beta subunit. The T cell receptor beta gene can refer to NCBI Gene ID number 6957.

As used herein, the term “detectable cell surface expression of an endogenous alpha/beta TCR” refers to the ability to detect one or more components of the TCR complex (e.g., an alpha/beta TCR complex) on the cell surface of a T cell (e.g., a CAR T cell), or a population of T cells (e.g., CAR T cells) described herein, using standard experimental methods. Such methods can include, for example, immunostaining and/or flow cytometry specific for components of the TCR itself, such as a TCR alpha or TCR beta chain, or for components of the assembled cell surface TCR complex, such as CD3. Methods for detecting cell surface expression of an endogenous TCR (e.g., an alpha/beta TCR) on an immune cell include those described in MacLeod et al. (2017) Molecular Therapy 25(4): 949-961.

As used herein, the term “no detectable cell surface expression of CD3” refers to lack of detection of CD3 on the surface of a T cell (e.g., a CAR T cell) described herein, or population of T cells (e.g., CAR T cells) described herein, as detected using standard experimental methods in the art. Methods for detecting cell surface expression of CD3 on an immune cell include those described in MacLeod et al. (2017).

As used herein, the terms “exogenous T cell receptor” or “exogenous TCR” refer to a TCR whose sequence is introduced into the genome of an immune effector cell (e.g., a human T cell) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest. In some examples, exogenous TCRs can include an extracellular ligand-binding domain comprising an antibody, or antibody fragment, having specificity for a target antigen. Such an antibody fragment can be, for example, a single-chain variable fragment (scFv).

As used herein, the term “antibody” refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

As used herein, the terms “antigen” or “Ag” refers to a molecule that is capable of being bound specifically by an antibody, or otherwise provokes an immune response. This immune response may involve either antibody production, or the activation of specific immunologically-competent cells, or both.

As used herein, the terms “tumor associated antigen” or “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present disclosure are derived from, cancers including but not limited to primary or metastatic melanoma, thymoma, lymphoma, sarcoma, lung cancer, liver cancer, NHL, leukemias, uterine cancer, cervical cancer, bladder cancer, kidney cancer and adenocarcinomas such as breast cancer, prostate cancer, ovarian cancer, pancreatic cancer, and the like.

As used herein, the term “monoclonal antibody” refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, the term “anti-CD52 antibody” refers to an antibody, or antibody fragment or conjugate, having specificity for a CD52 protein expressed on the cell surface of human T cells. In some examples, an anti-CD52 antibody can be a monoclonal antibody. In some cases, an anti-CD52 antibody can be alemtuzumab (i.e., CAMPATH). In some cases, an anti-CD52 antibody can be ALLO-647 (Allogene Therapeutics, San Francisco, Calif.).

As used herein, the term “anti-CD3 antibody” refers to an antibody, or antibody fragment or conjugate, having specificity for a CD3 protein expressed on the cell surface of human T cells. In some examples, an anti-CD3 antibody can be a monoclonal antibody. In some cases, an anti-CD3 antibody can be muromonab-CD3 (Orthoclone OKT3™), otelixizumab, teplizumab, foralumab, visilizumab, or derivatives thereof which have specificity for CD3.

As used herein, the term “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain or moiety, a transmembrane domain, and an intracellular domain, wherein the intracellular domain comprises one or more signaling domains and/or co-stimulatory domains.

In some embodiments, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

The intracellular stimulatory domain includes one or more cytoplasmic signaling domains that transmit an activation signal to the T cell following antigen binding. Such cytoplasmic signaling domains can include, without limitation, a CD3 zeta signaling domain (e.g., and without limitation, SEQ ID NO: 16).

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such TRAF binding-domains may include, for example, those set forth in SEQ ID NOs: 9-11. Such intracellular co-stimulatory domains can be any of those known in the art and can include, without limitation, those co-stimulatory domains disclosed in WO 2018/067697 including, for example, Novel 6 (“N6”; SEQ ID NO: 12). Further examples of co-stimulatory domains can include 4-1BB (CD137; SEQ ID NO: 13), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain (SEQ ID NO: 15). Alternatively, the transmembrane domain can be synthetic and can comprise predominantly hydrophobic residues such as leucine and valine.

The hinge region refers to any oligo or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRllla receptor or IgGl. In certain examples, the hinge region can be a CD8 alpha domain (SEQ ID NO: 14).

As used herein, the term “chimeric antigen receptor T cell” or “CAR T cell” refers to a human T cell modified to comprise a transgene encoding a CAR, wherein the CAR is expressed on the cell surface of the T cell.

As used herein, the term “proliferate in vivo” refers to an expansion in the number of genetically-modified human immune cells described herein in a subject following administration during immunotherapy. Such proliferation or expansion can be determined by methods known in the art and those shown in the examples herein, which include, for example, utilizing PCR analysis to determine the number of copies of a transgene (e.g., a CAR or exogenous TCR transgene) per mg of DNA isolated from peripheral blood mononuclear cells over a time course following administration of the pharmaceutical composition comprising the genetically-modified human immune cells.

As used herein, the term “cancer” should be understood to encompass any neoplastic disease (whether invasive or metastatic) which is characterized by abnormal and uncontrolled cell division causing malignant growth or tumor.

As used herein, the term “cancer of B-cell origin” refers to any blood cancer that affects immature and/or mature B lymphocytes.

As used herein, the term “multiple myeloma” refers to a cancer affecting plasma cells.

As used herein, the term “acute lymphoblastic leukemia” or “ALL” refers to a cancer of the lymphoid line of blood cells characterized by the development of large numbers of immature lymphocytes.

As used herein, the term “non-Hodgkin lymphoma” or “NHL” refers to a group of blood cancers that includes all types of lymphoma except Hodgkin's lymphomas.

As used herein, the term “chronic lymphocytic leukemia” or “CLL” refers to a type of non-Hodgkin lymphoma cancer characterized by the clonal proliferation and accumulation of neoplastic B lymphocytes in the blood and bone marrow.

As used herein, the term “small lymphocytic leukemia” or “SLL” refers to a type of non-Hodgkin lymphoma cancer characterized by the clonal proliferation and accumulation of neoplastic B lymphocytes in the lymph nodes, and spleen.

As used herein, the term “mantle cell lymphoma” or “MCL” refers to a type of non-Hodgkin lymphoma cancer characterized by a CD5 positive antigen-naive pre-germinal center B-cell within the mantle zone that surrounds normal germinal center follicles. MCL cells generally over-express cyclin D1 due to a chromosomal translocation in the DNA.

As used herein, the term “diffuse large B cell lymphoma” or “DLBCL” refers to a non-Hodgkin lymphoma affecting B cells that can develop in the lymph nodes or in extranodal sites (areas outside the lymph nodes) such as the gastrointestinal tract, testes, thyroid, skin, breast, bone, brain, or essentially any organ of the body.

As used herein, the term “response,” “complete response,” “complete response with incomplete blood count recovery,” “refractory disease,” “partial response,” “disease progression” or “progressive disease,” “refractory disease,” “relapse” or “relapsed disease” each refer to assessments of disease state and response in subjects following treatment according to the methods disclosed herein. For example, the response criteria for the assessment of subjects with B-ALL are based on the NCCN Guidelines (NCCN, 2017). As described therein, a complete response is defined as no circulating blasts or extramedullary disease, no lymphadenopathy, splenomegaly, skin/gum infiltration/testicular mass/CNS involvement, trilineage hematopoiesis and <5% blasts, absolute neutrophil count (ANC)>1000/mm³, platelets >100,000/mm³, and no recurrence for 4 weeks. Complete response with incomplete blood count recovery (CRi) is defined as meeting all criteria for complete response except platelet count and/or ANC. An overall response rate (ORR) can be calculated as CR+CRi. Refractory disease can be defined as failure to achieve a complete response at the end of induction. Progressive disease can be defined as an increase of at least 25% in the absolute number of circulating or bone marrow blasts or development of extramedullary disease. Relapsed disease can be defined as the reappearance of blasts in the blood or bone marrow (>5%) or in any extramedullary site after a complete response. For NHL, response criteria for local and central assessments of subjects with NHL are based on the revised Lugano Classification (Cheson et al, 2016), which incorporates PET-CT. A complete response (i.e., a complete metabolic response) is characterized by lymph nodes and extralymphatic sites having a score of 1, 2, or 3 with or without a residual mass on 5-point scale (5PS), and it is recognized that in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. A complete response is further characterized by no new lesions and no evidence of fluorodeoxyglucose (FDG)-avid disease in marrow. A partial response (i.e., partial metabolic response) is characterized by lymph nodes and extralymphatic sites having a score of 4 or 5 with reduced uptake compared with Baseline and residual mass(es) of any size. At interim, these findings suggest responding disease. At end of treatment, these findings indicate residual disease. A partial response is further characterized by no new lesions and bone marrow wherein residual uptake is higher than uptake in normal marrow but reduced compared with baseline. No response or stable disease (i.e., no metabolic response) is characterized by target nodes/nodal masses and/or extranodal lesions having a score of 4 or 5 with no significant change in FDG uptake from baseline at interim or end of treatment, no new lesions, and no change in bone marrow from baseline. Progressive disease (i.e., progressive metabolic disease) is characterized by individual target nodes/nodal masses having a score 4 or 5 with an increase in intensity of uptake from baseline and/or new foci compatible with lymphoma, new FDG-avid foci consistent with lymphoma at interim or end-of-treatment assessment, no non-measured lesions, new FDG-avid foci consistent with lymphoma rather than another etiology (e.g., infection, inflammation), and new or recurrent FDG-avid foci. RECIL 2017 criteria can also be used to assess response based on assessment of target lesions. A complete response is characterized by complete disappearance of all target lesions and all nodes with long axis <10 mm, >30% decrease in the sum of longest diameters of target lesions (PR) with normalization of FDG-PET, normalization of FDG-PET (Deauville score 1-3), no involvement of bone marrow, and no new lesions. A partial response is characterized by >30% decrease in the sum of longest diameters of target lesions but not a complete response, a positive FDG-PET (Deauville score 4-5), any bone marrow involvement, and no new lesions. A minor response is characterized by >10% decrease in the sum of longest diameters of target lesions but not a PR (<30%), any FDG-PET, any bone marrow involvement, and no new lesions. Stable disease is characterized by <10% decrease or <20% increase in the sum of longest diameters of target lesions, any FDG-PET, any bone marrow involvement, and no new lesions. Progressive disease is characterized by >20% increase in the sum of longest diameters of target lesions, for small lymph nodes measuring <15 mm post therapy, a minimum absolute increase of 5 mm and the long diameter should exceed 15 mm, appearance of a new lesion, any FDG-PET, any bone marrow involvement, and the appearance of new lesions or no new lesions.

As used herein, the terms “nuclease” and “endonuclease” are used interchangeably to refer to naturally-occurring or engineered enzymes, which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI (SEQ ID NO: 18), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit—Linker—C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, 51 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, Stsl, Hhal, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, End1, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the term “megaTAL” refers to a single-chain endonuclease comprising a transcription activator-like effector (TALE) DNA binding domain with an engineered, sequence-specific homing endonuclease.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, 51 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and Stsl restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the terms “target site” or “target sequence” refers to a region of the chromosomal DNA of a cell comprising a recognition sequence for a nuclease.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “homology arms” or “sequences homologous to sequences flanking a nuclease cleavage site” refer to sequences flanking the 5′ and 3′ ends of a nucleic acid molecule, which promote insertion of the nucleic acid molecule into a cleavage site generated by a nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome. In some embodiments, the homology arms are about 500 base pairs.

As used herein, the terms “template nucleic acid,” “donor template,” or “repair template” refer to a nucleic acid sequence that is desired to be inserted into a cleavage site within a cell's genome. Such template nucleic acids or donor templates can comprise, for example, a transgene, such as an exogenous transgene, which encodes a protein of interest (e.g., a CAR). The template nucleic acid or donor template can comprise 5′ and 3′ homology arms having homology to 5′ and 3′ sequences, respectively, that flank a cleavage site in the genome where insertion of the template is desired. Insertion can be accomplished, for example, by homology-directed repair (HDR).

As used herein, the term “transgene” refers to a nucleic acid molecule that encodes a polypeptide or RNA that is heterologous to the vector sequences flanking the coding sequence or is intended for transfer or has been transferred to a non-native cell or genomic locus.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention.

As used herein, the term “endogenous” in reference to a nucleotide sequence or protein is intended to mean a sequence or protein that is naturally comprised within or expressed by a cell.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein, the term with respect to recombinant proteins, the term “modification” means any insertion, deletion, or substitution of an amino acid residue in the recombinant sequence relative to a reference sequence (e.g., a wild-type or a native sequence).

As used herein, the term “inactivation” or “inactivated” or “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated inactivation or disruption of a gene can result in the expression of a truncated protein and/or expression of a protein that does not retain its wild-type function. Additionally, introduction of a donor template into a gene can result in no expression of an encoded protein, expression of a truncated protein, and/or expression of a protein that does not retain its wild-type function.

As used herein, the term with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymo1.266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to a virus (i.e., a viral vector). Viruses can include, without limitation retroviruses, lentiviruses, adenoviruses, and adeno-associated viruses (AAVs).

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values 0 and 2 if the variable is inherently continuous.

### 2.1 Principle of the Invention

The present invention provides for therapeutic methods for reducing the number of target cells, such as diseased or infected cells, in a subject. In such methods, the subject is administered genetically-modified immune cells comprising a chimeric antigen receptor (CAR) or exogenous T cell receptor (TCR), wherein the CAR or exogenous TCR comprises an extracellular ligand-binding domain having specificity for an antigen on the target cells. Administration of the pharmaceutical composition is preceded by the administration of a lymphodepletion regimen and/or immunosuppression regimen that allows for a more persistent and stronger therapeutic effect. The lymphodepletion regimen serves to reduce the subject's endogenous lymphocytes and can be administered to the subject prior to the administration of the pharmaceutical composition comprising the genetically-modified human immune cells.

In some embodiments, the immunosuppression regimen comprises administering an mTOR (mammalian target of rapamycin) inhibitor. Sirolimus is a non-limiting example of an inhibitor of mTOR. Unlike calcineurin inhibitors, sirolimus and other mTOR inhibitors have a lower risk of adverse events, particularly related to renal function. Because sirolimus is mechanistically linked to the suppression of IL-2 and cytokine receptor-dependent signaling, it blocks both T and B cell activation. However, because these signaling domains are constitutively activated in, for example, a CAR T cell through CAR binding to a target antigen, it should have minimal effect on those cell signaling pathways while inhibiting natural T cells (i.e., patient T cells) that would otherwise reject allogeneic donor cells through normally expressed signaling domains. In total, there may be some suppression of the expansion of allogeneic cells (e.g., CAR T or CAR NK cells), but the prevention of rejection would result in a net effect of improved clinical outcomes. Notably, sirolimus also has some anti-tumor effects in CD19 expressing cancers, which may further improve the clinical benefit observed with this combination.

### 2.2 Lymphodepletion Regimen

In some embodiments of the presently disclosed methods, the methods comprise a lymphodepletion regimen wherein one or more effective doses of one or more lymphodepletion agents are administered to the subject in order to reduce the number of endogenous lymphocytes prior to administration of the pharmaceutical composition. The one or more lymphodepletion agents used in the presently disclosed methods can be biological lymphodepletion agents, chemotherapeutic lymphodepletion agents, or a combination thereof.

A biological lymphodepletion agent can be, for example, any biological material, such an antibody, antibody fragment, antibody conjugate, or the like, that can be administered as part of a lymphodepletion regimen to reduce endogenous lymphocytes in the subject for immunotherapy. Such biological lymphodepletion agents can include, for example, a monoclonal antibody, or a fragment thereof. In some examples, the biological lymphodepletion agent has specificity for a T cell antigen; i.e., an antigen expressed on the cell surface of T cells. Examples of such antigens include, without limitation, CD52 and CD3. In a particular example, the biological lymphodepletion agent is an antibody, such as a monoclonal antibody, having specificity for CD52. Such antibodies can include, for example, alemtuzumab (i.e., CAMPATH), ALLO-647 (Allogene Therapeutics, San Francisco, Calif.), derivatives thereof, which bind CD52, or any other CD52 antibody. In another particular example, the biological lymphodepletion agent is an antibody, such as a monoclonal antibody, having specificity for CD3. In some cases, an anti-CD3 antibody can be muromonab-CD3 (Orthoclone OKT3™), otelixizumab, teplizumab, foralumab, visilizumab, or derivatives thereof, which have specificity for CD3.

Lymphodepletion regimens of the invention can include the administration of one or more chemotherapeutic lymphodepletion agents. Pre-treatment or pre-conditioning patients prior to cell therapies with one or more chemotherapeutic lymphodepletion agents improves the efficacy of the cellular therapy by reducing the number of endogenous host lymphocytes in the subject, thereby providing a more optimal environment for administered cells to proliferate once administered to the subject. An effective dose of one or more chemotherapeutic lymphodepletion agents can result in the reduction of one or more endogenous lymphocytes (e.g., B cells, T cells, and/or NK cells) in the subject by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control; e.g., relative to a starting amount in the subject undergoing treatment, relative to a pre-determined threshold, or relative to an untreated subject.

In some embodiments, 1, 2, 3, 4, or more chemotherapeutic lymphodepletion agents may be included in the lymphodepletion regimen.

Chemotherapeutic lymphodepletion agents can refer to non-biological materials, such as small molecules, that can be administered as part of a lymphodepletion regimen to reduce endogenous lymphocytes in the subject for immunotherapy. In some examples, the chemotherapeutic lymphodepleting agent can be lymphodepleting but non-myeloablative. Chemotherapeutic lymphodepletion agents can include those known in the art including, without limitation, purine analogs (such as fludarabine, pentostatin, azathioprine, mercaptopurine such as 6-mercaptopurine, clofarabine, cladribine, and thiopurines such as thioguanine), and compounds capable of inducing interstrand cross-links within DNA (such as cisplatin, mitomycin C, carmustine, psoralen or nitrogen mustard-derived alkylating agents like cyclophosphamide, ifosfamide, chlorambucil, uramustine, melphalan, and bendamustine). Other non-limiting examples of chemotherapeutic lymphodepletion agents useful in the presently disclosed methods include daunorubicin, L-asparaginase, methotrexate, prednisone, dexamethasone, and nelarabine. In some embodiments, the chemotherapeutic lymphodepletion agent is cyclophosphamide. In some embodiments, the chemotherapeutic lymphodepletion agent is pentostatin.

The lymphodepletion regimen administered during the method of the invention can be administered in an amount effective (i.e., an effective dose) to deplete or reduce the quantity of endogenous lymphocytes in the subject, for example, by 1%, 5%, 10%, 20%, 30%, 40%, 50%, 60%, 70%, 80%, 90%, 91%, 92%, 93%, 94%, 95%, 96%, 97%, 98%, 99%, or more, relative to a control, e.g., relative to a starting amount in the subject undergoing treatment, relative to a pre-determined threshold, or relative to an untreated subject, prior to administration of the pharmaceutical composition. The reduction in lymphocyte count can be monitored using conventional techniques known in the art, such as by flow cytometry analysis of cells expressing characteristic lymphocyte cell surface antigens in a blood sample withdrawn from the subject at varying intervals during treatment with the antibody. According to some embodiments, when the concentration of lymphocytes has reached a minimum value in response to the lymphodepletion regimen, the physician may conclude the lymphodepletion therapy and may begin preparing the subject for administration of the pharmaceutical composition.

In various embodiments, the one or more chemotherapeutic lymphodepletion agents can be administered one day to one month (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days) prior to administration of the pharmaceutical compositions described herein. In some embodiments, a chemotherapeutic lymphodepletion agent is administered to the subject three or more days prior to administration of the second pharmaceutical composition. In certain embodiments, administration of a chemotherapeutic lymphodepletion agent ends at least one day, at least two days, or at least three days prior to administration of the second pharmaceutical composition.

In some embodiments, a chemotherapeutic lymphodepletion agent can be administered as a single dose per day on each of eight consecutive days, as a single dose per day on each of seven consecutive days, as a single dose per day on each of six consecutive days, as a single dose per day on each of five consecutive days, as a single dose per day on each of four consecutive days, as a single dose per day on each of three consecutive days, as a single dose per day on each of two consecutive days, or as a single dose on one day, prior to administration of the pharmaceutical composition.

In some of these embodiments, the lymphodepletion regimen comprises administering an alkylating agent (e.g., cyclophosphamide) at a dose of about 1 to about 200 mg/kg/day, about 1 to about 100 mg/kg/day, about 10 to about 200 mg/kg/day, about 10 to about 100 mg/kg/day, about 20 to about 200 mg/kg/day, or about 20 to about 100 mg/kg/day. In some of these embodiments, the lymphodepletion regimen comprises administering an alkylating agent (e.g., cyclophosphamide) at a dose of about 1, about 2, about 3, about 5, about 8, about 10, about 15, about 20, about 25, about 30, about 40, about 45, about 50, about 55, about 60, about 70, about 75, about 80, about 90, about 95, about 100, about 110, about 120, about 130, about 140, about 150, about 160, about 170, about 180, about 190, or about 200 mg/kg/day. In particular embodiments, the lymphodepletion regimen comprises administering an alkylating agent (e.g., cyclophosphamide) at a dose of about 50 mg/kg/day.

In certain embodiments, the lymphodepletion regimen comprises administering an alkylating agent (e.g., cyclophosphamide) once daily starting 8 days and ending 2 days prior to administration of the second pharmaceutical composition, starting 8 days and ending 1 day prior to administration of the second pharmaceutical composition, starting 7 days and ending 2 days prior to administration of the second pharmaceutical composition, starting 6 days and ending 2 days prior to administration of the second pharmaceutical composition, starting 5 days and ending 2 days prior to administration of the second pharmaceutical composition, starting 5 days and ending 3 days prior to administration of the second pharmaceutical composition, starting 4 days and ending 2 days prior to administration of the second pharmaceutical composition, starting 3 days and ending 2 days prior to administration of the second pharmaceutical composition, starting 3 days and ending 1 day prior to administration of the second pharmaceutical composition, or starting 2 days and ending 1 day prior to administration of the second pharmaceutical composition. In some of these embodiments, the lymphodepletion regimen comprises administering an alkylating agent (e.g., cyclophosphamide) once daily starting 8 days prior to administration of the second pharmaceutical composition and ending 7 days, 6 days, 5 days, 4 days, 3 days, 2 days, or 1 day prior to administration of the second pharmaceutical composition. In other embodiments, the lymphodepletion regimen comprises administering an alkylating agent (e.g., cyclophosphamide) once daily starting 7 days prior to administration of the second pharmaceutical composition and ending 6 days, 5 days, 4 days, 3 days, 2 days, or 1 day prior to administration of the second pharmaceutical composition. In still other embodiments, the lymphodepletion regimen comprises administering an alkylating agent (e.g., cyclophosphamide) once daily starting 6 days prior to administration of the second pharmaceutical composition and ending 5 days, 4 days, 3 days, 2 days, or 1 day prior to administration of the second pharmaceutical composition. In yet other embodiments, the lymphodepletion regimen comprises administering an alkylating agent (e.g., cyclophosphamide) once daily starting 5 days prior to administration of the second pharmaceutical composition and ending 4 days, 3 days, 2 days, or 1 day prior to administration of the second pharmaceutical composition. In other embodiments, the lymphodepletion regimen comprises administering an alkylating agent (e.g., cyclophosphamide) once daily starting 4 days prior to administration of the second pharmaceutical composition and ending 3 days, 2 days, or 1 day prior to administration of the second pharmaceutical composition. In still other embodiments, the lymphodepletion regimen comprises administering an alkylating agent (e.g., cyclophosphamide) once daily starting 3 days prior to administration of the second pharmaceutical composition and ending 2 days or 1 day prior to administration of the second pharmaceutical composition. In yet other embodiments, the lymphodepletion regimen comprises administering an alkylating agent (e.g., cyclophosphamide) once daily starting 2 days prior to administration of the second pharmaceutical composition and ending 1 day prior to administration of the second pharmaceutical composition.

In particular embodiments, the lymphodepletion regimen comprises administering cyclophosphamide once daily starting 5 days prior to administration of the second pharmaceutical composition and ending 2 days prior to administration of the second pharmaceutical composition. In other particular embodiments, the lymphodepletion regimen comprises administering cyclophosphamide once daily starting 3 days prior to administration of the second pharmaceutical composition and ending 2 days prior to administration of the second pharmaceutical composition.

The one or more lymphodepletion agents can be administered to the subject using any acceptable route of administration. In some of those embodiments wherein the lymphodepletion agent comprises an alkylating agent (e.g., cyclophosphamide), an alkylating agent (e.g., cyclophosphamide) is administered orally.

In particular embodiments, the lymphodepletion regimen comprises administering an alkylating agent (e.g., cyclophosphamide) once daily at a dose of about 1 to about 200 mg/kg/day, about 1 to about 100 mg/kg/day, about 10 to about 200 mg/kg/day, about 10 to about 100 mg/kg/day, about 20 to about 200 mg/kg/day, or about 20 to about 100 mg/kg/day, including but not limited to about 1, about 2, about 3, about 5, about 8, about 10, about 15, about 20, about 25, about 30, about 40, about 45, about 50, about 55, about 60, about 70, about 75, about 80, about 90, about 95, about 100, about 110, about 120, about 130, about 140, about 150, about 160, about 170, about 180, about 190, or about 200 mg/kg/day starting 8 days and ending 2 days prior to administration of the second pharmaceutical composition, starting 8 days and ending 1 day prior to administration of the second pharmaceutical composition, starting 7 days and ending 2 days prior to administration of the second pharmaceutical composition, starting 6 days and ending 2 days prior to administration of the second pharmaceutical composition, starting 5 days and ending 2 days prior to administration of the second pharmaceutical composition, starting 4 days and ending 2 days prior to administration of the second pharmaceutical composition, starting 3 days and ending 2 days prior to administration of the second pharmaceutical composition, starting 3 days and ending 1 day prior to administration of the second pharmaceutical composition, or starting 2 days and ending 1 day prior to administration of the second pharmaceutical composition. In some of these embodiments, the lymphodepletion regimen comprises administering about 50 mg/kg/day of an alkylating agent (e.g., cyclophosphamide) once daily starting 3 days and ending 2 days prior to administration of the second pharmaceutical composition. In certain embodiments, the lymphodepletion regimen comprises administering about 50 mg/kg/day of an alkylating agent (e.g., cyclophosphamide) once daily starting 5 days and ending 2 days prior to administration of the second pharmaceutical composition.

In some embodiments, the lymphodepletion regimen does not comprise administering an effective dose of a biological lymphodepletion agent. In some embodiments, the lymphodepletion regimen does not comprise administering a biological lymphodepletion agent. Non-limiting examples of a biological lymphodepletion agent include monoclonal antibodies or fragments thereof. Such monoclonal antibodies or fragments thereof can have specificity for a T cell antigen. In some embodiments, the monoclonal antibody or fragment thereof is an anti-CD52 monoclonal antibody or fragment thereof, or an anti-CD3 antibody or fragment thereof. In certain embodiments, the monoclonal antibody is alemtuzumab or ALLO-647. In some embodiments, the lymphodepletion regimen includes administration of a biological lymphodepletion agent in an amount no greater than 1.0 mg/kg during the 7 day period preceding administration of the pharmaceutical composition. In some embodiments, the lymphodepletion regimen includes administration of a biological lymphodepletion agent in an amount no greater than 0.75 mg/kg, 0.5 mg/kg, 0.25 mg/kg, or 0.1 mg/kg during the 7 day period preceding administration of the pharmaceutical composition. In certain embodiments, the lymphodepletion regimen includes administration of a biological lymphodepletion agent in an amount no greater than 0.1 mg/kg during the 7 day period preceding administration of the pharmaceutical composition. In some examples, the lymphodepletion regimen includes no more than a minimal effective dose of a biological lymphodepletion agent.

In some embodiments, the lymphodepletion regimen comprises administering one or more effective doses of a purine analog (e.g., pentostatin, fludarabine) and a compound capable of inducing interstrand cross-links within DNA (e.g., cyclophosphamide).

In certain embodiments of the method, the compound capable of inducing interstrand cross-links within DNA is cyclophosphamide. In some embodiments, the lymphodepletion regimen comprises administering cyclophosphamide at a dose of about 50 to about 500 mg/day, about 100 to about 300 mg/day, about 50 to about 300 mg/day, about 100 to about 500 mg/day, or about 150 to about 250 mg/day. In some of these embodiments, the lymphodepletion regimen comprises administering cyclophosphamide at a dose of about 50, about 100, about 150, about 175, about 180, about 185, about 190, about 195, about 200, about 205, about 210, about 215, about 220, about 225, about 250, about 300, about 350, about 400, about 450, or about 500 mg/day. In particular embodiments, the lymphodepletion regimen comprises administering cyclophosphamide at a dose of about 200 mg/day.

In certain embodiments, the lymphodepletion regimen comprises administering cyclophosphamide once daily starting 11 days prior to administration of the pharmaceutical composition and ending 1 day prior to administration of the pharmaceutical composition, starting 9 days prior to administration of the pharmaceutical composition and ending 1 day prior to administration of the pharmaceutical composition, starting 7 days prior to administration of the pharmaceutical composition and ending 1 day prior to administration of the pharmaceutical composition, starting 11 days prior to administration of the pharmaceutical composition and ending 2 days prior to administration of the pharmaceutical composition, starting 9 days prior to administration of the pharmaceutical composition and ending 2 days prior to administration of the pharmaceutical composition, starting 7 days prior to administration of the pharmaceutical composition and ending 2 days prior to administration of the pharmaceutical composition, starting 11 days prior to administration of the pharmaceutical composition and ending 3 days prior to administration of the pharmaceutical composition, starting 9 days prior to administration of the pharmaceutical composition and ending 3 days prior to administration of the pharmaceutical composition, or starting 7 days prior to administration of the pharmaceutical composition and ending 3 days prior to administration of the pharmaceutical composition.

In some embodiments, the lymphodepletion regimen comprises administering an alkylating agent (e.g., cyclophosphamide) once daily starting 11 days prior to administration of the pharmaceutical composition and ending 10 days, 9 days, 8 days, 7 days, 6 days, 5 days, 4 days, 3 days, 2 days, or 1 day prior to administration of the pharmaceutical composition. In other embodiments, the lymphodepletion regimen comprises administering an alkylating agent (e.g., cyclophosphamide) once daily starting 10 days prior to administration of the pharmaceutical composition and ending 9 days, 8 days, 7 days, 6 days, 5 days, 4 days, 3 days, 2 days, or 1 day prior to administration of the pharmaceutical composition. In still other embodiments, the lymphodepletion regimen comprises administering an alkylating agent (e.g., cyclophosphamide) once daily starting 9 days prior to administration of the pharmaceutical composition and ending 8 days, 7 days, 6 days, 5 days, 4 days, 3 days, 2 days, or 1 day prior to administration of the pharmaceutical composition. In yet other embodiments, the lymphodepletion regimen comprises administering an alkylating agent (e.g., cyclophosphamide) once daily starting 8 days prior to administration of the pharmaceutical composition and ending 7 days, 6 days, 5 days, 4 days, 3 days, 2 days, or 1 day prior to administration of the pharmaceutical composition. In other embodiments, the lymphodepletion regimen comprises administering an alkylating agent (e.g., cyclophosphamide) once daily starting 7 days prior to administration of the pharmaceutical composition and ending 6 days, 5 days, 4 days, 3 days, 2 days, or 1 day prior to administration of the pharmaceutical composition. In still other embodiments, the lymphodepletion regimen comprises administering an alkylating agent (e.g., cyclophosphamide) once daily starting 6 days prior to administration of the pharmaceutical composition and ending 5 days, 4 days, 3 days, 2 days, or 1 day prior to administration of the pharmaceutical composition. In yet other embodiments, the lymphodepletion regimen comprises administering an alkylating agent (e.g., cyclophosphamide) once daily starting 5 days prior to administration of the pharmaceutical composition and ending 4 days, 3 days, 2 days, or 1 day prior to administration of the pharmaceutical composition. In other embodiments, the lymphodepletion regimen comprises administering an alkylating agent (e.g., cyclophosphamide) once daily starting 4 days prior to administration of the pharmaceutical composition and ending 3 days, 2 days, or 1 day prior to administration of the pharmaceutical composition. In still other embodiments, the lymphodepletion regimen comprises administering an alkylating agent (e.g., cyclophosphamide) once daily starting 3 days prior to administration of the pharmaceutical composition and ending 2 days or 1 day prior to administration of the pharmaceutical composition. In yet other embodiments, the lymphodepletion regimen comprises administering an alkylating agent (e.g., cyclophosphamide) once daily starting 2 days prior to administration of the pharmaceutical composition and ending 1 day prior to administration of the pharmaceutical composition.

In particular embodiments, the lymphodepletion regimen comprises administering cyclophosphamide once daily starting 7 days prior to administration of the pharmaceutical composition and ending 1 day prior to administration of the pharmaceutical composition.

In some embodiments, an alkylating agent (e.g., cyclophosphamide) is administered orally.

In some of these embodiments, the lymphodepletion regimen comprises administering an alkylating agent (e.g., cyclophosphamide) once daily at a dose of about 50 to about 500 mg/day, about 100 to about 300 mg/day, about 50 to about 300 mg/day, about 100 to about 500 mg/day, or about 150 to about 250 mg/day, including but not limited to about 50, about 100, about 150, about 175, about 180, about 185, about 190, about 195, about 200, about 205, about 210, about 215, about 220, about 225, about 250, about 300, about 350, about 400, about 450, or about 500 mg/day starting 11 days, 10 days, 9 days, 8 day, 7 days, 6 days, 5 days, 4 days, 3 days, or 2 days prior to administration of the pharmaceutical composition and ending 1 day prior to administration of the pharmaceutical composition, starting 11 days, 10 days, 9 days, 8 day, 7 days, 6 days, 5 days, 4 days, or 3 days prior to administration of the pharmaceutical composition and ending 2 days prior to administration of the pharmaceutical composition, or starting 11 days, 10 days, 9 days, 8 day, 7 days, 6 days, 5 days, or 4 days prior to administration of the pharmaceutical composition and ending 3 days prior to administration of the pharmaceutical composition In particular embodiments, the lymphodepletion regimen comprises administering about 200 mg of an alkylating agent (e.g., cyclophosphamide) orally once daily starting 7 days prior to administration of the pharmaceutical composition and ending 1 day prior to administration of the pharmaceutical composition.

In some embodiments, the lymphodepletion regimen comprises administering a purine analog (e.g., pentostatin) at a dose of about 1 to about 30 mg/m²/day, about 1 to about 20 mg/m²/day, about 1 to about 10 mg/m²/day, about 2 to about 20 mg/m²/day, or about 2 to about 10 mg/m²/day. In some of these embodiments, the lymphodepletion regimen comprises administering a purine analog (e.g., pentostatin) at a dose of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mg/m²/day. In particular embodiments, the lymphodepletion regimen comprises administering a purine analog (e.g., pentostatin) at a dose of about 4 mg/m²/day.

In certain embodiments, the lymphodepletion regimen comprises administering a purine analog (e.g., pentostatin) once daily starting 12 days prior to administration of the pharmaceutical composition and ending 1 day prior to administration of the pharmaceutical composition, starting 9 days prior to administration of the pharmaceutical composition and ending 1 day prior to administration of the pharmaceutical composition, starting 7 days prior to administration of the pharmaceutical composition and ending 1 day prior to administration of the pharmaceutical composition, starting 12 days prior to administration of the pharmaceutical composition and ending 2 days prior to administration of the pharmaceutical composition, starting 9 days and ending 2 days, starting 7 days prior to administration of the pharmaceutical composition and ending 2 days prior to administration of the pharmaceutical composition, starting 12 days and ending 3 days, starting 9 days prior to administration of the pharmaceutical composition and ending 3 days prior to administration of the pharmaceutical composition, starting 7 days prior to administration of the pharmaceutical composition and ending 3 days prior to administration of the pharmaceutical composition, or starting 9 prior to administration of the pharmaceutical composition days and ending 3 days prior to administration of the pharmaceutical composition, prior to administration of the pharmaceutical composition.

In some of these embodiments, the lymphodepletion regimen comprises administering a purine analog (e.g., pentostatin) once daily starting 12 days prior to administration of the pharmaceutical composition and ending 11 days, 10 days, 9 days, 8 days, 7 days, 6 days, 5 days, 4 days, 3 days, 2 days, or 1 day prior to administration of the pharmaceutical composition. In some of these embodiments, the lymphodepletion regimen comprises administering a purine analog (e.g., pentostatin) once daily starting 11 days prior to administration of the pharmaceutical composition and ending 10 days, 9 days, 8 days, 7 days, 6 days, 5 days, 4 days, 3 days, 2 days, or 1 day prior to administration of the pharmaceutical composition. In other embodiments, the lymphodepletion regimen comprises administering a purine analog (e.g., pentostatin) once daily starting 10 days prior to administration of the pharmaceutical composition and ending 9 days, 8 days, 7 days, 6 days, 5 days, 4 days, 3 days, 2 days, or 1 day prior to administration of the pharmaceutical composition. In still other embodiments, the lymphodepletion regimen comprises administering a purine analog (e.g., pentostatin) once daily starting 9 days prior to administration of the pharmaceutical composition and ending 8 days, 7 days, 6 days, 5 days, 4 days, 3 days, 2 days, or 1 day prior to administration of the pharmaceutical composition. In yet other embodiments, the lymphodepletion regimen comprises administering a purine analog (e.g., pentostatin) once daily starting 8 days prior to administration of the pharmaceutical composition and ending 7 days, 6 days, 5 days, 4 days, 3 days, 2 days, or 1 day prior to administration of the pharmaceutical composition. In other embodiments, the lymphodepletion regimen comprises administering a purine analog (e.g., pentostatin) once daily starting 7 days prior to administration of the pharmaceutical composition and ending 6 days, 5 days, 4 days, 3 days, 2 days, or 1 day prior to administration of the pharmaceutical composition. In still other embodiments, the lymphodepletion regimen comprises administering a purine analog (e.g., pentostatin) once daily starting 6 days prior to administration of the pharmaceutical composition and ending 5 days, 4 days, 3 days, 2 days, or 1 day prior to administration of the pharmaceutical composition. In yet other embodiments, the lymphodepletion regimen comprises administering a purine analog (e.g., pentostatin) once daily starting 5 days prior to administration of the pharmaceutical composition and ending 4 days, 3 days, 2 days, or 1 day prior to administration of the pharmaceutical composition. In other embodiments, the lymphodepletion regimen comprises administering a purine analog (e.g., pentostatin) once daily starting 4 days prior to administration of the pharmaceutical composition and ending 3 days, 2 days, or 1 day prior to administration of the pharmaceutical composition. In still other embodiments, the lymphodepletion regimen comprises administering a purine analog (e.g., pentostatin) once daily starting 3 days prior to administration of the pharmaceutical composition and ending 2 days or 1 day prior to administration of the pharmaceutical composition. In yet other embodiments, the lymphodepletion regimen comprises administering a purine analog (e.g., pentostatin) once daily starting 2 days prior to administration of the pharmaceutical composition and ending 1 day prior to administration of the pharmaceutical composition.

In other embodiments, the lymphodepletion regimen comprises administering a purine analog (e.g., pentostatin) once daily every 4 days, every 3 days, or every 2 days. In some such embodiments, the lymphodepletion regimen comprises administering a purine analog (e.g., pentostatin) once daily every 4 days, every 3 days, or every 2 days starting 15 days, 14 days, 13 days, 12 days, 11 days, 10 days, 9 days, 8 days, 7 days, 6 days, 5 days, 4 days, 3 days, or 2 days prior to administration of the pharmaceutical composition, such that a purine analog (e.g., pentostatin) is not administered on the same day as, or following administration of, the pharmaceutical composition. In some of these embodiments, the lymphodepletion regimen comprises administering a purine analog (e.g., pentostatin) once daily every 3 days starting 15 days prior to administration of the pharmaceutical composition and ending 3 days prior to administration of the pharmaceutical composition, starting 14 days prior to administration of the pharmaceutical composition and ending 2 days prior to administration of the pharmaceutical composition, starting 13 days prior to administration of the pharmaceutical composition and ending 1 day prior to administration of the pharmaceutical composition, starting 12 days prior to administration of the pharmaceutical composition and ending 3 days prior to administration of the pharmaceutical composition, starting 11 days prior to administration of the pharmaceutical composition and ending 2 days prior to administration of the pharmaceutical composition, starting 10 days prior to administration of the pharmaceutical composition and ending 1 day prior to administration of the pharmaceutical composition, starting 9 days prior to administration of the pharmaceutical composition and ending 3 days prior to administration of the pharmaceutical composition, starting 8 days prior to administration of the pharmaceutical composition and ending 2 days prior to administration of the pharmaceutical composition, starting 7 days prior to administration of the pharmaceutical composition and ending 1 day prior to administration of the pharmaceutical composition, starting 6 days prior to administration of the pharmaceutical composition and ending 3 days prior to administration of the pharmaceutical composition, starting 5 days prior to administration of the pharmaceutical composition and ending 2 days prior to administration of the pharmaceutical composition, or starting 4 days prior to administration of the pharmaceutical composition and ending 1 day prior to administration of the pharmaceutical composition. In particular embodiments, the lymphodepletion regimen comprises administering a purine analog (e.g., pentostatin) once daily every 3 days, starting 9 days and ending 3 days prior to administration of the pharmaceutical composition.

In some embodiments, a purine analog (e.g., pentostatin) is administered intravenously.

In some of these embodiments, the lymphodepletion regimen comprises administering a purine analog (e.g., pentostatin) once daily at a dose of about 1 to about 30 mg/m²/day, about 1 to about 20 mg/m²/day, about 1 to about 10 mg/m²/day, about 2 to about 20 mg/m²/day, or about 2 to about 10 mg/m²/day, including but not limited to about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mg/m²/day every 3 days starting 15 days prior to administration of the pharmaceutical composition and ending 3 days prior to administration of the pharmaceutical composition, starting 14 days prior to administration of the pharmaceutical composition and ending 2 days prior to administration of the pharmaceutical composition, starting 13 days prior to administration of the pharmaceutical composition and ending 1 day prior to administration of the pharmaceutical composition, starting 12 days prior to administration of the pharmaceutical composition and ending 3 days prior to administration of the pharmaceutical composition, starting 11 days prior to administration of the pharmaceutical composition and ending 2 days prior to administration of the pharmaceutical composition, starting 10 days prior to administration of the pharmaceutical composition and ending 1 day prior to administration of the pharmaceutical composition, starting 9 days prior to administration of the pharmaceutical composition and ending 3 days prior to administration of the pharmaceutical composition, starting 8 days prior to administration of the pharmaceutical composition and ending 2 days prior to administration of the pharmaceutical composition, starting 7 days prior to administration of the pharmaceutical composition and ending 1 day prior to administration of the pharmaceutical composition, starting 6 days prior to administration of the pharmaceutical composition and ending 3 days prior to administration of the pharmaceutical composition, starting 5 days prior to administration of the pharmaceutical composition and ending 2 days prior to administration of the pharmaceutical composition, or starting 4 days prior to administration of the pharmaceutical composition and ending 1 day prior to administration of the pharmaceutical composition. In particular embodiments, the lymphodepletion regimen comprises administering about 4 mg/m²/day of a purine analog (e.g., pentostatin) once daily every 3 days starting 9 days and ending 3 days prior to administration of the pharmaceutical composition.

In particular embodiments, the lymphodepletion regimen comprises administering about 200 mg/day an alkylating agent (e.g., cyclophosphamide) orally once daily starting 7 days and ending 1 day prior to administration of the pharmaceutical composition, and administering about 4 mg/m²/day a purine analog (e.g., pentostatin) intravenously every 3 days starting 9 days and ending 3 days prior to administration of the pharmaceutical composition.

In some embodiments, the lymphodepletion regimen does not comprise administering an effective dose of a biological lymphodepletion agent. In some embodiments, the lymphodepletion regimen does not comprise administering a biological lymphodepletion agent. Non-limiting examples of a biological lymphodepletion agent include monoclonal antibodies or fragments thereof. Such monoclonal antibodies or fragments thereof can have specificity for a T cell antigen. In some embodiments, the monoclonal antibody or fragment thereof is an anti-CD52 monoclonal antibody or fragment thereof, or an anti-CD3 antibody or fragment thereof. In certain embodiments, the monoclonal antibody is alemtuzumab or ALLO-647. In some embodiments, the lymphodepletion regimen includes administration of a biological lymphodepletion agent in an amount no greater than 1.0 mg/kg during the 7 day period preceding administration of the pharmaceutical composition. In some embodiments, the lymphodepletion regimen includes administration of a biological lymphodepletion agent in an amount no greater than 0.75 mg/kg, 0.5 mg/kg, 0.25 mg/kg, or 0.1 mg/kg during the 7 day period preceding administration of the pharmaceutical composition. In certain embodiments, the lymphodepletion regimen includes administration of a biological lymphodepletion agent in an amount no greater than 0.1 mg/kg during the 7 day period preceding administration of the pharmaceutical composition.

### 2.3 Immunosuppression Regimens

In some embodiments of the presently disclosed methods, the immunotherapeutic methods utilize an immunosuppression regimen wherein one or more effective doses of one or more immunosuppressant agents are administered to a subject, in order to suppress the subject's immune response to administered genetically-modified human immune cells. The immunosuppression regimen can be administered before, during, and/or after administration of the pharmaceutical composition comprising the genetically-modified human immune cells. Any immunosuppressant agent may be used in the presently disclosed methods, but in some embodiments, the immunosuppression regimen does not comprise administering a cytotoxic agent or an effective dose thereof. In certain embodiments, the immunosuppressant agent that is administered to a subject is an mTOR inhibitor. In some of these embodiments, the mTOR inhibitor is sirolimus.

The immunosuppressant can be administered to the subject after the genetically-modified human immune cells are administered to ensure continued immunosuppression and persistence of the genetically-modified human immune cells. The immunosuppressant can continue to be administered indefinitely, however, due to the negative side effects of continued immunosuppression, in some embodiments administration of the one or more immunosuppressant agents is halted after about 32 days after administration of the pharmaceutical composition comprising the genetically-modified human immune cells.

The administration of sirolimus results in an effective serum concentration of sirolimus in the subject, which in some embodiments is about 15 ng/mL to about 30 ng/mL, about 20 ng/mL to about 30 ng/mL, about 15 ng/mL to about 20 ng/mL, or about 15 ng/mL to about 25 ng/mL. In some of these embodiments, the effective dose of sirolimus results in an effective serum concentration of sirolimus in the subject of about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, or about 30 ng/mL. In particular embodiments, the effective dose of sirolimus results in an effective serum concentration of sirolimus in the subject of about 20 ng/mL.

In order to achieve the effective serum concentration of sirolimus, sirolimus can be administered once daily or if the effective serum concentration is desired to be achieved more rapidly, sirolimus may first be administered to the subject as a loading dose, followed by once daily maintenance doses.

In some embodiments, sirolimus is administered once daily starting 12 days, 11 days, 10 days, 9 days, 8 days, 7 days, 6 days, 5 days, 4 days, 3 days, 2 days, or 1 day, prior to administration of the pharmaceutical composition. In particular embodiments, sirolimus is administered once daily staring 1 day prior to administration of the pharmaceutical composition. In certain embodiments, sirolimus is administered once on the day of administration of the pharmaceutical composition, and once daily each day thereafter ending 14 days, 15 days, 16 days, 17 days, 18 days, 19 days, 20 days, 21 days, 22 days, 23 days, 24 days, 25 days, 26 days, 27 days, 28 days, or longer, after administration of the pharmaceutical composition. In particular embodiments, sirolimus is administered once on the day of administration of the pharmaceutical composition and once daily each day thereafter ending 21 days after administration of the pharmaceutical composition.

In some embodiments, sirolimus is administered orally.

In some of those embodiments wherein the presently disclosed methods further comprise administering an effective dose of sirolimus, the subject is first administered one loading dose of sirolimus, followed by administration of a maintenance dose of sirolimus once daily in order to achieve the effective serum concentration of sirolimus, wherein the once daily maintenance dose of sirolimus begins the day after the loading dose of sirolimus is administered to the subject.

In some of these embodiments, the loading dose of sirolimus is about 5 mg to about 25 mg, about 5 mg to about 20 mg, about 5 mg to about 18 mg, about 5 mg to about 16 mg, about 8 mg to about 25 mg, about 8 mg to about 20 mg, about 8 mg to about 18 mg, about 8 mg to about 16 mg, about 10 mg to about 25 mg, about 10 mg to about 20 mg, about 10 mg to about 18 mg, about 10 mg to about 16 mg, about 12 mg to about 25 mg, about 12 mg to about 20 mg, about 12 mg to about 18 mg, or about 12 mg to about 16 mg. In some of these embodiments, the loading dose of sirolimus is about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg. In particular embodiments, the loading dose of sirolimus is about 16 mg. In some embodiments, the maintenance dose of sirolimus is about 1 mg to about 15 mg, about 1 mg to about 10 mg, about 1 mg to about 8 mg, about 1 mg to about 5 mg, about 2 mg to about 15 mg, about 2 mg to about 10 mg, about 2 mg to about 8, mg, about 2 mg to about 5 mg, about 3 mg to about 15 mg, about 3 mg to about 10 mg, about 3 mg to about 8 mg, or about 3 mg to about 5 mg. In some of these embodiments, the maintenance dose of sirolimus is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg. In particular embodiments, the maintenance dose of sirolimus is about 4 mg. In certain embodiments, the loading dose of sirolimus is about 5 mg to about 25 mg, about 5 mg to about 20 mg, about 8 mg to about 20 mg, about 8 mg to about 18 mg, about 10 mg to about 18 mg, or about 12 mg to about 18 mg, including but not limited to about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, and the maintenance dose of sirolimus is about 1 mg to about 15 mg, about 1 mg to about 10 mg, about 2 mg to about 8, mg, or about 3 mg to about 5 mg, including but not limited to about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg. In some of these embodiments, the loading dose of sirolimus is about 16 mg and the maintenance dose of sirolimus is about 4 mg.

In some embodiments, the loading dose of sirolimus is administered once 8 days, 7 days, 6 days, 5 days, 4 days, 3 days, 2 days, or 1 day prior to administration of the pharmaceutical composition, and the maintenance dose is administered once daily beginning one day after the loading dose is administered. In certain embodiments, following administration of the loading dose, the maintenance dose is administered once daily until 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day after administration of the pharmaceutical composition. In some of these embodiments, a loading dose is administered 1 day prior to administration of the pharmaceutical composition, and a maintenance dose of sirolimus is administered once daily starting beginning on the same day as administration of the pharmaceutical composition, and ending 21 days after administration of the pharmaceutical composition.

In certain embodiments, the immunosuppressant agent, such as sirolimus, can be administered orally. In particular embodiments, the loading dose of sirolimus is administered orally. In some embodiments, the maintenance dose is administered orally.

### 2.4 Human Immune Cells and Populations of Genetically-Modified Human Immune Cells

The invention provides methods that utilize genetically-modified human immune cells and populations thereof and provides methods for producing the same. In some embodiments, the genetically-modified human immune cells used in the presently disclosed methods are human immune cells. In some embodiments, the immune cells are T cells, or cells derived therefrom. In other embodiments, the immune cells are natural killer (NK) cells, or cells derived therefrom. In still other embodiments, the immune cells are B cells, or cells derived therefrom. In yet other embodiments, the immune cells are monocyte or macrophage cells or cells derived therefrom.

Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines, NK cell lines, B cell lines, monocyte cells lines, or macrophage cell lines available in the art may be used. In some embodiments of the present disclosure, immune cells are obtained from a unit of blood collected from a subject using any number of techniques known to the skilled artisan. In one embodiment, cells from the circulating blood of an individual are obtained by apheresis. In further examples, immune cells useful for the methods can be derived from induced pluripotent stem cells (iPSCs) that have been differentiated into immune cells.

Generally, a CAR of the present disclosure will comprise at least an extracellular domain, a transmembrane domain, and an intracellular domain. In some embodiments, the extracellular domain comprises a target-specific binding element otherwise referred to as an extracellular ligand-binding domain or moiety. In some embodiments, the intracellular domain, or cytoplasmic domain, comprises at least one co-stimulatory domain and one or more signaling domains.

Thus, a CAR or exogenous TCR useful in the invention comprises an extracellular ligand-binding domain. The choice of ligand-binding domain depends upon the type and number of ligands that define the surface of a target cell. For example, the ligand-binding domain may be chosen to recognize a ligand that acts as a cell surface marker on target cells associated with a particular disease state. Thus, some examples of cell surface markers that may act as ligands for the ligand-binding domain in a CAR or exogenous TCR can include those associated with viruses, bacterial and parasitic infections, autoimmune disease, and cancer cells. In some embodiments, a CAR or exogenous TCR is engineered to target a cancer-specific antigen of interest by way of engineering a desired ligand-binding moiety that specifically binds to an antigen on a cancer (i.e., tumor) cell. In the context of the present disclosure, “cancer antigen,” tumor antigen,” “cancer-specific antigen,” or “tumor-specific antigen” refer to antigens that are common to specific hyperproliferative disorders such as cancer.

In some embodiments, the extracellular ligand-binding domain of the CAR or exogenous TCR is specific for any antigen or epitope of interest, particularly any cancer antigen or epitope of interest. As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79B, IL1RAP, glypican 3 (GPC3), CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, insulin growth factor (IGF1)-1, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the Al domain of tenascin-C (TnC Al) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), CS1, or a virus-specific surface antigen such as an HIV-specific antigen (such as HIV gp120); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor or exogenous TCR can also comprise an autoantigen (see, Payne et al. (2016), Science 353 (6295): 179-184), that can be recognized by autoantigen-specific B cell receptors on B lymphocytes, thus directing genetically-modified human immune cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor or exogenous TCR can also comprise a naturally-occurring ligand for an antigen of interest, or a fragment of a naturally-occurring ligand which retains the ability to bind the antigen of interest.

In certain embodiments, the ligand-binding domain of the CAR or exogenous TCR is an scFv. In some such embodiments, the scFv comprises a heavy chain variable (VH) domain and a light chain variable (VL) domain from a monoclonal antibody having specificity for a cancer cell antigen. In some examples, the scFv comprises a VH domain and a VL domain obtained from a CD19-specific antibody. In certain examples, the VH domain comprises SEQ ID NO: 3 and the VL domain comprises SEQ ID NO: 4. In some such examples, the CAR can comprise an amino acid sequence having at least 80%, at least 85%, at least 90%, at least 95%, or more, sequence identity to SEQ ID NO: 5 wherein the CAR has specificity for CD19. In some embodiments, the CAR comprises SEQ ID NO: 5. In some examples, the scFv comprises a VH domain and a VL domain obtained from a CD20-specific antibody. In certain examples, the VH domain comprises SEQ ID NO: 6 and the VL domain comprises SEQ ID NO: 7. In some such examples, the CAR can comprise an amino acid sequence having at least 80%, at least 85%, at least 90%, at least 95%, or more, sequence identity to SEQ ID NO: 8 wherein the CAR has specificity for CD20. In some embodiments, the CAR comprises SEQ ID NO: 8. In some examples, the scFv comprises a VH domain and a VL domain obtained from a BCMA-specific antibody.

A CAR can comprise a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain (SEQ ID NO: 15). Alternatively, the transmembrane domain can be synthetic and can comprise predominantly hydrophobic residues such as leucine and valine.

The hinge region refers to any oligo or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRllla receptor or IgGl. In certain examples, the hinge region can be a CD8 alpha domain (SEQ ID NO: 14).

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular signaling domain can include one or more cytoplasmic signaling domains that transmit an activation signal to the T cell following antigen binding. Such cytoplasmic signaling domains can include, without limitation, a CD3 zeta signaling domain (SEQ ID NO: 16).

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such TRAF binding-domains may include, for example, those set forth in SEQ ID NOs: 9-11. Such intracellular co-stimulatory domains can be any of those known in the art and can include, without limitation, those co-stimulatory domains disclosed in WO 2018/067697 including, for example, Novel 6 (“N6”; SEQ ID NO: 12). Further examples of co-stimulatory domains can include 4-1BB (CD137; SEQ ID NO: 13), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain.

In other embodiments, the genetically-modified human immune cell comprises a nucleic acid sequence encoding an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest. In some examples, the extracellular ligand-binding domain of an exogenous TCR can comprise an antibody or antibody fragment, such as an scFv, fused to one of the TCR complex subunits.

The CARs or exogenous TCRs described herein can have, for example, specificity for cancer cell antigens. Such cancers can include, without limitation, carcinoma, lymphoma, sarcoma, blastomas, leukemia, cancers of B cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin lymphoma. In specific embodiments, cancers and disorders include but are not limited to pre-B ALL (pediatric indication), adult ALL, mantle cell lymphoma, diffuse large B cell lymphoma, salvage post allogenic bone marrow transplantation, and the like. These cancers can be treated using a combination of CARs that target, for example, CD19, CD20, CD22, and/or ROR1. In some non-limiting examples, a genetically-modified human immune cell or population thereof of the present disclosure targets carcinomas, lymphomas, sarcomas, melanomas, blastomas, leukemias, and germ cell tumors, including but not limited to cancers of B-cell origin, neuroblastoma, osteosarcoma, prostate cancer, renal cell carcinoma, liver cancer, gastric cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, multiple myeloma, Hodgkin lymphoma, non-Hodgkin lymphomas, acute myeloid lymphoma, chronic myelogenous leukemia, chronic lymphoid leukemia, immunoblastic large cell lymphoma, acute lymphoblastic leukemia, mycosis fungoides, anaplastic large cell lymphoma, and T-cell lymphoma, and any combinations of said cancers. In certain embodiments, cancers of B-cell origin include, without limitation, B-lineage acute lymphoblastic leukemia, B-cell chronic lymphocytic leukemia, B-cell lymphoma, diffuse large B cell lymphoma, pre-B ALL (pediatric indication), mantle cell lymphoma, follicular lymphoma, marginal zone lymphoma, Burkitt's lymphoma, multiple myeloma, and B-cell non-Hodgkin lymphoma. In some examples, cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

In some embodiments, genetically-modified human immune cells useful in the presently disclosed methods comprise an inactivated TCR alpha gene and/or an inactivated TCR beta gene. Inactivation of the TCR alpha gene and/or TCR beta gene to generate the genetically-modified human immune cells used in the present invention occurs in at least one or both alleles where the TCR alpha gene and/or TCR beta gene is being expressed. Accordingly, inactivation of one or both genes prevents expression of the endogenous TCR alpha chain or the endogenous TCR beta chain protein. Expression of these proteins is required for assembly of the endogenous alpha/beta TCR on the cell surface. Thus, inactivation of the TCR alpha gene and/or the TCR beta gene results in genetically-modified human immune cells that have no detectable cell surface expression of the endogenous alpha/beta TCR. The endogenous alpha/beta TCR incorporates CD3. Therefore, cells with an inactivated TCR alpha gene and/or TCR beta chain can have no detectable cell surface expression of CD3. In particular embodiments, the inactivated gene is a TCR alpha constant region (TRAC) gene.

In some examples, the TCR alpha gene, the TRAC gene, or the TCR beta gene is inactivated by insertion of a transgene encoding the CAR or exogenous TCR. Insertion of the CAR or exogenous TCR transgene disrupts expression of the endogenous TCR alpha chain or TCR beta chain and, therefore, prevents assembly of an endogenous alpha/beta TCR on the T cell surface. In some examples, the CAR or exogenous TCR transgene is inserted into the TRAC gene. In a particular example, a CAR or exogenous TCR transgene is inserted into the TRAC gene at an engineered meganuclease recognition sequence comprising SEQ ID NO: 1. In particular examples, the CAR or exogenous TCR transgene is inserted into SEQ ID NO: 1 between nucleotide positions 13 and 14.

As used herein, “detectable cell surface expression of an endogenous alpha/beta TCR” refers to the ability to detect one or more components of the TCR complex (e.g., an alpha/beta TCR complex) on the cell surface of an immune cell using standard experimental methods. Such methods can include, for example, immunostaining and/or flow cytometry specific for components of the TCR itself, such as a TCR alpha or TCR beta chain, or for components of the assembled cell surface TCR complex, such as CD3. Methods for detecting cell surface expression of an endogenous TCR (e.g., an alpha/beta TCR) on an immune cell include those described in the examples herein, and, for example, those described in MacLeod et al. (2017).

Similarly, “detectable cell surface expression of CD3” refers to lack of detection of CD3 on the surface of an immune cell (e.g., a CAR T cell) described herein, or population of immune cells (e.g., CAR T cells) described herein, as detected using standard experimental methods in the art. Methods for detecting cell surface expression of CD3 on an immune cell include those described in MacLeod et al. (2017).

Human immune cells used in the present invention may require activation prior to introduction of a nuclease and/or an exogenous sequence of interest to generate CAR T cells. For example, human immune cells can be contacted with anti-CD3 and anti-CD28 antibodies that are soluble or conjugated to a support (e.g., beads) for a period of time sufficient to activate the cells.

Immune cells used in the invention can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

The invention utilizes a population of human immune cells that includes a plurality of genetically-modified human immune cells expressing a cell surface CAR or exogenous TCR. In various embodiments of the invention, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified human immune cell as described herein. In a particular example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are genetically-modified human immune cells that express a CAR or exogenous TCR and have an inactivated TCR alpha and/or beta gene.

### 2.5 Methods for Genetically Modifying Human Immune Cells

The present invention uses human immune cells, or populations of human immune cells comprising a plurality of genetically-modified human immune cells that have been modified to express a CAR or an exogenous TCR. Such human immune cells can be modified in a number of ways in order to introduce a transgene encoding a CAR or exogenous TCR into the genome of the cell, such that the CAR or exogenous TCR is expressed by the cell. For example, a transgene encoding a CAR or exogenous TCR can be introduced into the genome of an immune cell by random integration. In some such cases, the transgene can be randomly integrated by transducing the cell with a lentivirus comprising the transgene. In other examples, a transgene encoding a CAR or exogenous TCR can be introduced by targeted insertion at a specified location in the genome. In some such cases, targeted integration can be achieved by use of a site-specific, engineered nuclease that generates a cleavage site at a particular location in the genome (e.g., within a target gene), and insertion of a donor template encoding the transgene into the cleavage site.

In some examples of the invention, the genetically-modified human immune cells comprise an inactivated TCR alpha gene and/or an inactivated TCR beta gene. In particular examples, the inactivated gene can be a TCR alpha constant region (TRAC) gene. Such gene inactivations can disrupt expression of the endogenous TCR alpha chain and/or the endogenous TCR beta chain, which are each necessary for the assembly of the endogenous alpha/beta TCR. Thus, inactivation of one or more of these genes results in genetically-modified human immune cells that do not have detectable cell surface express of an endogenous alpha/beta TCR and, in some embodiments, do not have detectable cell surface expression of CD3 which is part of the TCR complex.

In some examples, inactivation of the TCR alpha gene, TCR beta gene, and/or the TRAC gene can result from the insertion of a transgene into one of these endogenous genes. Insertion of the transgene disrupts expression of the polypeptide encoded by the gene; e.g., the endogenous TCR alpha chain or the endogenous TCR beta chain. In some examples, the transgene encodes the CAR or exogenous TCR, which is expressed by the cell and localized to the cell surface.

Insertion of the donor template comprising the CAR or exogenous TCR transgene can be achieved by use of an engineered nuclease to generate a cleavage site within a recognition sequence in the genome, such as within the TCR alpha gene, the TRAC gene, or the TCR beta gene.

The use of nucleases for disrupting expression of an endogenous TCR gene has been disclosed, including the use of zinc finger nucleases (ZFNs), transcription activator-like effector nucleases (TALENs), megaTALs, and CRISPR systems (e.g., Osborn et al. (2016), Molecular Therapy 24(3): 570-581; Eyquem et al. (2017), Nature 543: 113-117; U.S. Pat. No. 8,956,828; U.S. Publication No. US2014/0301990; U.S. Publication No. US2012/0321667). The specific use of engineered meganucleases for cleaving DNA targets in the human TRAC gene has also been previously disclosed. For example, International Publication No. WO 2014/191527, which disclosed variants of the I-OnuI meganuclease that were engineered to target a recognition sequence within exon 1 of the TCR alpha constant region gene. Moreover, in International Publication Nos. WO 2017/062439 and WO 2017/062451, Applicants disclosed engineered meganucleases which have specificity for recognition sequences in exon 1 of the TCR alpha constant region gene. These included “TRC 1-2 meganucleases” which have specificity for the TRC 1-2 recognition sequence (SEQ ID NO: 1) in exon 1 of the TRAC gene. The '439 and '451 publications also disclosed methods for targeted insertion of a CAR coding sequence or an exogenous TCR coding sequence into a cleavage site in the TCR alpha constant region gene.

Any engineered nuclease can be used for targeted insertion of the donor template, including an engineered meganuclease, a zinc finger nuclease, a TALEN, a compact TALEN, a CRISPR system nuclease, or a megaTAL.

For example, zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a Compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). A CRISPR system comprises two components: (1) a CRISPR nuclease; and (2) a short “guide RNA” comprising a ˜20 nucleotide targeting sequence that directs the nuclease to a location of interest in the genome. The CRISPR system may also comprise a tracrRNA. By expressing multiple guide RNAs in the same cell, each having a different targeting sequence, it is possible to target DNA breaks simultaneously to multiple sites in the genome.

Engineered meganucleases that bind double-stranded DNA at a recognition sequence that is greater than 12 base pairs can be used for the presently disclosed methods. A meganuclease can be an endonuclease that is derived from I-CreI and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker.

Nucleases referred to as megaTALs are single-chain endonucleases comprising a transcription activator-like effector (TALE) DNA binding domain with an engineered, sequence-specific homing endonuclease.

The CAR or exogenous TCR transgene can be inserted at any position within the TCR alpha gene, the TCR beta gene, or the TRAC gene, such that insertion of the transgene results in disrupted expression of the endogenous polypeptide; i.e., the endogenous TCR alpha chain or the endogenous TCR beta chain. In some examples, the CAR or exogenous TCR transgene can be inserted in the TRAC gene at a meganuclease recognition sequence comprising SEQ ID NO: 1. In particular examples, the transgene is inserted between positions 13 and 14 of SEQ ID NO: 1.

In particular embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. For example, nuclease-mediated insertion using engineered single-chain meganucleases has been disclosed in International Publication Nos. WO 2017/062439 and WO 2017/062451. Nuclease-mediated insertion of the donor template can also be accomplished using an engineered single-chain meganuclease comprising SEQ ID NO: 17.

In some embodiments, mRNA encoding the engineered nuclease is delivered to the cell because this reduces the likelihood that the gene encoding the engineered nuclease will integrate into the genome of the cell.

The mRNA encoding an engineered nuclease can be produced using methods known in the art such as in vitro transcription. In some embodiments, the mRNA comprises a modified 5′ cap. Such modified 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, Calif.), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element. The mRNA may contain modifications of naturally-occurring nucleosides to nucleoside analogs. Any nucleoside analogs known in the art are envisioned for use in the present methods. Such nucleoside analogs can include, for example, those described in U.S. Pat. No. 8,278,036. In particular embodiments, nucleoside modifications can include a modification of uridine to pseudouridine, and/or a modification of uridine to N1-methyl pseudouridine.

Purified nuclease proteins can be delivered into cells to cleave genomic DNA, which allows for homologous recombination or non-homologous end-joining at the cleavage site with an exogenous nucleic acid molecule encoding a polypeptide of interest as described herein, by a variety of different mechanisms known in the art, including those further detailed herein.

In another particular embodiment, a nucleic acid encoding an engineered nuclease can be introduced into the cell using a single-stranded DNA template. The single-stranded DNA can further comprise a 5′ and/or a 3′ AAV inverted terminal repeat (ITR) upstream and/or downstream of the sequence encoding the engineered nuclease. In other embodiments, the single-stranded DNA can further comprise a 5′ and/or a 3′ homology arm upstream and/or downstream of the sequence encoding the engineered nuclease.

In other embodiments, genes encoding a nuclease of the invention are introduced into a cell using a linearized DNA template. Such linearized DNA templates can be produced by methods known in the art. For example, a plasmid DNA encoding a nuclease can be digested by one or more restriction enzymes such that the circular plasmid DNA is linearized prior to being introduced into a cell.

Purified engineered nuclease proteins, or nucleic acids encoding engineered nucleases, can be delivered into cells to cleave genomic DNA by a variety of different mechanisms known in the art, including those further detailed herein below.

In some embodiments, the nuclease proteins, or DNA/mRNA encoding the nuclease, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, engineered nucleases, or DNA/mRNA encoding nucleases, are coupled covalently or non-covalently to an antibody that recognizes a specific cell-surface receptor expressed on target cells such that the nuclease protein/DNA/mRNA binds to and is internalized by the target cells. Alternatively, engineered nuclease protein/DNA/mRNA can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

In some embodiments, nuclease proteins, or DNA/mRNA encoding nucleases, are encapsulated within biodegradable hydrogels for injection or implantation within the desired region of the liver (e.g., in proximity to hepatic sinusoidal endothelial cells or hematopoietic endothelial cells, or progenitor cells which differentiate into the same). Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, nuclease proteins, or DNA/mRNA encoding nucleases, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nuclease proteins, mRNA, or DNA can be attached to or encapsulated with the nanoparticle core. This increases the copy number of the protein/mRNA/DNA that is delivered to each cell and, so, increases the intracellular expression of each nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the nuclease proteins or DNA/mRNA encoding the nucleases are encapsulated within liposomes or complexed using cationic lipids (see, e.g., LIPOFECTAMINE™, Life Technologies Corp., Carlsbad, Calif.; Zuris et al. (2015) Nat Biotechnol. 33: 73-80; Mishra et al. (2011) J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, nuclease proteins, or DNA/mRNA encoding nucleases, are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, nuclease proteins, or DNA/mRNA encoding nucleases, are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, nuclease proteins, or DNA/mRNA encoding nucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, nuclease proteins, or DNA/mRNA encoding nucleases, are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability, reduce nonspecific interactions, and enhance cell-specific targeting and drug release.

In some embodiments, genes encoding a nuclease are delivered using a virus. Such viruses are known in the art and include retroviruses, lentiviruses, adenoviruses, and adeno-associated viruses (AAVs) (reviewed in Vannucci, et al. (2013 New Microbiol. 36:1-22). AAVs useful in the invention can have any serotype that allows for transduction of the virus into a target cell type and expression of the nuclease gene in the target cell. In particular embodiments, AAVs have a serotype of AAV2 or AAV6. AAVs can be single-stranded AAVs or alternatively, can be self-complementary such that they do not require second-strand DNA synthesis in the host cell (McCarty, et al. (2001) Gene Ther. 8:1248-54).

If the nuclease genes are delivered in DNA form (e.g. plasmid) and/or via a virus (e.g. AAV) they must be operably linked to a promoter. In some embodiments, this can be a viral promoter such as endogenous promoters from the virus (e.g. the LTR of a lentiviral vector) or the well-known cytomegalovirus- or SV40 virus-early promoters. In a preferred embodiment, nuclease genes are operably linked to a promoter that drives gene expression preferentially in the target cell. In some examples, nuclease genes are operably linked to a synthetic promoter, such as a JeT promoter (U.S. Pat. No. 6,555,674).

The donor template (e.g., a template nucleic acid) comprising the CAR or exogenous TCR transgene is inserted into a cleavage site in the targeted genes. In some embodiments, the donor template comprises a 5′ homology arm and a 3′ homology arm flanking the transgene and elements of the insert. Such homology arms have sequence homology to corresponding sequences 5′ upstream and 3′ downstream of the nuclease recognition sequence where a cleavage site is produced. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

The transgene encoding the CAR or exogenous TCR can further comprise additional control sequences. For example, the sequence can include homologous recombination enhancer sequences, Kozak sequences, polyadenylation sequences, transcriptional termination sequences, selectable marker sequences (e.g., antibiotic resistance genes), origins of replication, and the like. Sequences encoding engineered nucleases can also include at least one nuclear localization signal. Examples of nuclear localization signals are known in the art (see, e.g., Lange et al., J. Biol. Chem., 2007, 282:5101-5105).

A donor template comprising the CAR or exogenous TCR transgene can be introduced into the cell by any of the means previously discussed. In a particular embodiment, the donor template is introduced by way of a virus, such as a recombinant AAV. AAVs useful for introducing an exogenous nucleic acid can have any serotype that allows for transduction of the virus into the cell and insertion of the exogenous nucleic acid sequence into the cell genome. In particular embodiments, the AAVs have a serotype of AAV2 or AAV6. AAVs can be single-stranded AAVs or, alternatively, can be self-complementary such that they do not require second-strand DNA synthesis in the host cell. In certain embodiments, the transgene for the CAR or the exogenous TCR is operably-linked to a promoter such as, for example, a JeT promoter.

In another particular embodiment, the donor template encoding the CAR or exogenous TCR transgene can be introduced into the cell using a single-stranded DNA template. The single-stranded DNA can comprise the exogenous sequence of interest and, in preferred embodiments, can comprise 5′ and 3′ homology arms to promote insertion of the nucleic acid sequence into the cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, the donor template encoding the CAR transgene can be introduced into the cell by transfection with a linearized DNA template. In some examples, a plasmid DNA can be digested by one or more restriction enzymes such that the circular plasmid DNA is linearized prior to transfection into the cell.

### 2.6 Pharmaceutical Compositions

The method of the invention comprises administering a pharmaceutical composition comprising a population of human immune cells, including a plurality of genetically-modified human immune cells. Such pharmaceutical compositions can be prepared in accordance with known techniques. See, e.g., Remington, The Science and Practice of Pharmacy (21st ed. 2005). In the manufacture of a pharmaceutical formulation according to the invention, cells are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions used in the invention can further comprise one or more additional agents useful in the treatment of a disease in the subject. In additional embodiments, pharmaceutical compositions of the invention can further include biological molecules, such as cytokines (e.g., IL-2, IL-7, IL-15, and/or IL-21), which may promote in vivo cell proliferation and engraftment of genetically-modified human immune cells. Pharmaceutical compositions comprising genetically-modified human immune cells used in the invention can be administered in the same composition as an additional agent or biological molecule or, alternatively, can be co-administered in separate compositions.

The present disclosure also provides genetically-modified human immune cells, or populations thereof, described herein for use as a medicament. The present disclosure further provides the use of genetically-modified human immune cells or populations thereof described herein in the manufacture of a medicament for treating a disease in a subject in need thereof. In one such aspect, the medicament is useful for cancer immunotherapy in subjects in need thereof.

### 2.7 Methods of Administering Populations of Human Immune Cells

The method of the invention comprises administering to a subject pharmaceutical composition comprising a population of human immune cells, wherein the population comprises a plurality of genetically-modified human immune cells. For example, the pharmaceutical composition administered to the subject can comprise an effective dose of genetically-modified human immune cells (e.g., CAR T cells or CAR NK cells) for treatment of a cancer or other disease and administration of the genetically-modified human immune cells of the invention represent an immunotherapy. The administered genetically-modified human immune cells are able to reduce the proliferation, reduce the number, or kill target cells in the recipient.

Unlike antibody therapies, genetically-modified human cells of the present disclosure are able to replicate and expand in vivo, resulting in long-term persistence that can lead to sustained control of a disease.

When an “effective amount” or “therapeutic amount” is indicated, the precise amount to be administered can be determined by a physician with consideration of individual differences in age, weight, disease state, tumor size (if present), extent of infection or metastasis, and condition of the patient (subject). In some embodiments, a pharmaceutical composition comprising the genetically-modified human immune cells or populations thereof described herein is administered at a dosage of 1×10⁴ to 1×10⁹ cells/kg body weight, including all integer values within those ranges. In further embodiments, the dosage is 1×10⁵ to 1×10⁷ cells/kg body weight, including all integer values within those ranges. Dosages of genetically-modified human immune cells can include any of the dosages described herein. In some embodiments, cell compositions are administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In some embodiments of the methods described herein, the pharmaceutical composition is administered at a dose of between about 1×10⁴ and about 1×10⁸, about 1×10⁴ and about 3×10⁶, about 1×10⁵ and about 1×10⁷, about 1×10⁵ and about 6×10⁶, about 1×10⁵ and about 3×10⁶, about 3×10⁵ and about 6×10⁶, and about 3×10⁵ and about 3×10⁶ genetically-modified human immune cells/kg. In some of these embodiments, the pharmaceutical composition is administered at a dose of about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ genetically-modified human immune cells/kg. In some of these embodiments, the pharmaceutical composition is administered at a dose of about 1×10⁶ genetically-modified human immune cells/kg. In some of these embodiments, the pharmaceutical composition is administered at a dose of about 2×10⁶ genetically-modified human immune cells/kg. In some of these embodiments, the pharmaceutical composition is administered at a dose of about 3×10⁶ genetically-modified human immune cells/kg. In some of these embodiments, the pharmaceutical composition is administered at a dose of about 4×10⁶ genetically-modified human immune cells/kg. In some of these embodiments, the pharmaceutical composition is administered at a dose of about 5×10⁶ genetically-modified human immune cells/kg. In some of these embodiments, the pharmaceutical composition is administered at a dose of about 6×10⁶ genetically-modified human immune cells/kg. In some of these embodiments, the pharmaceutical composition is administered at a dose of about 7×10⁶ genetically-modified human immune cells/kg. In some of these embodiments, the pharmaceutical composition is administered at a dose of about 8×10⁶ genetically-modified human immune cells/kg. In some of these embodiments, the pharmaceutical composition is administered at a dose of about 9×10⁶ genetically-modified human immune cells/kg. In some of these embodiments, the pharmaceutical composition is administered at a dose of about 1×10⁷ genetically-modified human immune cells/kg. In particular embodiments, the effective dose of the pharmaceutical composition comprises no more than 3×10⁸ genetically-modified human immune cells.

Examples of possible routes of administration of compositions comprising genetically-modified human immune cells include parenteral, (e.g., intravenous (IV), intramuscular (IM), intradermal, subcutaneous (SC), or infusion) administration. Examples of possible routes of administration of lymphodepletion regimens or immunosuppression regimens described herein include parenteral (e.g., intravenous (IV), intramuscular (IM), intradermal, subcutaneous (SC), or infusion) administration or oral administration. Moreover, the administration may be by continuous infusion or by single or multiple boluses. In specific embodiments, one or both of the agents is infused over a period of less than about 12 hours, less than about 10 hours, less than about 8 hours, less than about 6 hours, less than about 4 hours, less than about 3 hours, less than about 2 hours, or less than about 1 hour. In still other embodiments, the infusion occurs slowly at first and then is increased over time.

Pharmaceutical compositions of the invention can be useful for treating any disease state such as, for example, diseases that can be targeted by adoptive immunotherapy. In a particular embodiment, the presently disclosed methods are useful in the treatment of cancer. In some embodiments, the presently disclosed methods comprise administering a pharmaceutical composition comprising genetically-modified human immune cells targeting a cancer cell antigen (i.e., an antigen expressed on the surface of a cancer cell) for the purpose of treating cancer. Such cancers can include, without limitation, any of the cancers described herein.

In some embodiments, the presently disclosed methods reduce at least one symptom of a cancer. Symptoms of cancers are well known in the art and can be determined by known techniques. Further, the presently disclosed methods can reduce the number of cancer cells in a subject. Methods for determining the number of cancer cells in a subject vary based on the cancer being treated. Such methods are well known in the art and reductions in cancer cell numbers can be determined by known techniques.

In some of these embodiments wherein cancer is treated, the subject can be further administered an additional therapeutic agent or treatment, including, but not limited to gene therapy, radiation, surgery, or a chemotherapeutic agent(s) (i.e., chemotherapy).

### 2.8 Additional Doses of Pharmaceutical Compositions

In some embodiments of the method described herein, the methods comprise administering another dose of the pharmaceutical composition to the subject. In some such embodiments, the method comprises administering a second dose of the pharmaceutical composition without re-administration of the lymphodepletion regimen and/or the immunosuppression regimen (e.g., administration of sirolimus). In some such embodiments, the method comprises administering a second dose of the pharmaceutical composition 1, 2, 3, 4, 5, 6, 7, 8, 9, 10, 11, 12, 13, 14, 15, 16, 17, 18, 19, 20, or 21 days following administration of the first dose of the pharmaceutical composition. In certain embodiments, the method comprises administering a second dose of the pharmaceutical composition 10 days following administration of the first dose of the pharmaceutical composition. In some embodiments, the second dose of the pharmaceutical composition is administered at the same dose of genetically-modified human immune cells/kg as administered in the first dose of the pharmaceutical composition. In some embodiments, the second dose of the pharmaceutical composition is administered at a different dose of genetically-modified human immune cells/kg than administered in the first dose of the pharmaceutical composition.

In some embodiments, the method comprises administering a second dose of the pharmaceutical composition and a third dose of the pharmaceutical composition without re-administration of the lymphodepletion and/or immunosuppression (e.g., sirolimus) regimen. In some such embodiments, the method comprises administering a second dose of the pharmaceutical composition 1, 2, 3, 4, 5, 6, 7, 8, 9, 10, 11, 12, 13, 14, 15, 16, 17, 18, 19, 20, or 21 days following administration of the first dose of the pharmaceutical composition, and further comprises administering a third dose of the pharmaceutical composition 1, 2, 3, 4, 5, 6, 7, 8, 9, 10, 11, 12, 13, 14, 15, 16, 17, 18, 19, 20, or 21 days following administration of the second dose of the pharmaceutical composition. In some embodiments, the second dose of the pharmaceutical composition is administered 10 days following administration of the first dose of the pharmaceutical composition, and the third dose of the pharmaceutical composition is administered 4 days following administration of the second dose of the pharmaceutical composition. In some embodiments, the second dose of the pharmaceutical composition and the third dose of the pharmaceutical composition are each administered at the same dose of genetically-modified human immune cells/kg as administered in the first dose of the pharmaceutical composition. In some embodiments, the second dose of the pharmaceutical composition is administered at the same dose of genetically-modified human immune cells/kg as administered in the first dose of the pharmaceutical composition, and the third dose of the pharmaceutical composition is administered at a different dose of genetically-modified human immune cells/kg than administered in the first dose of the pharmaceutical composition. In some embodiments, the second dose of the pharmaceutical composition is administered at a different dose of genetically-modified human immune cells/kg than administered in the first dose of the pharmaceutical composition, and the third dose of the pharmaceutical composition is administered at the same dose of genetically-modified human immune cells/kg as administered in the first dose of the pharmaceutical composition. In some embodiments, the second dose of the pharmaceutical composition is administered at a different dose of genetically-modified human immune cells/kg than administered in the first dose of the pharmaceutical composition, and the third dose of the pharmaceutical composition is administered at the same dose of genetically-modified human immune cells/kg as administered in the first dose of the pharmaceutical composition. In some embodiments, the second dose of the pharmaceutical composition and the third dose of the pharmaceutical composition are administered at the same dose of genetically-modified human immune cells/kg.

In some embodiments of the methods described herein, the method comprises re-administration of the lymphodepletion and/or immunosuppression regimen (e.g., sirolimus), as well as the pharmaceutical composition to the subject. In some embodiments, re-administration occurs following a partial response or complete response with subsequent progressive disease. In some embodiments, re-administration occurs following no response and subsequent progressive disease. In some embodiments, re-administration occurs in subjects having a cancer that remains positive for the target cell antigen (e.g., cancer cell antigen) targeted by the genetically-modified human immune cells. In some embodiments, re-administration occurs about 2 weeks, 4 weeks, 6, weeks, 8 weeks, 10 weeks, 12, weeks, 14, weeks, 16 weeks, 18 weeks, 20 weeks, 22 weeks, 24 weeks, or more after the first administration. In some embodiments, the lymphodepletion and/or immunosuppression regimen (e.g., sirolimus) is re-administered at the same doses and/or schedule as the first administration. In some embodiments, the lymphodepletion and/or immunosuppression regimen (e.g., sirolimus) is re-administered at different doses and/or a different schedule as the first administration. In some embodiments, the pharmaceutical composition is re-administered at the same doses and/or schedule as the first administration. In some embodiments, the pharmaceutical composition is re-administered at different doses and/or a different schedule as the first administration. In certain embodiments, the pharmaceutical composition is re-administered at a higher dose than the first administration. In some embodiments, a first dose and a second dose, and optionally a third dose, of the pharmaceutical composition are re-administered according to any of the doses and dosing schedules described herein for administration of a first dose and a second dose, or for administration of a first dose, a second dose, and a third dose.

### 2.9 Variants

The present invention utilizes variants of the polypeptide and polynucleotide sequences described herein. As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein. Such variants may result, for example, from human manipulation. Biologically active variants of polypeptides described herein will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, as determined by sequence alignment programs and parameters described elsewhere herein. A biologically active variant of a polypeptide may differ from that polypeptide or subunit by as few as about 1-40 amino acid residues, as few as about 1-20, as few as about 1-10, as few as about 5, as few as 4, 3, 2, or even 1 amino acid residue.

The polypeptides may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode a polypeptide or RNA. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide (e.g., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its biological activity.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

Rapamycin prevents T cell responses directed against allogeneic CAR T cells

### 1. Methods

CAR T cells were produced using methods previously described (see MacLeod et al. (2017) Molecular Therapy 25(4): 949-961 and PCT International Patent Application no. PCT/US2019/027019). Briefly, T cells from a healthy donor were edited using TRC1-2L.1592 to disable the endogenous T cell receptor, and transduced with AAV7206 to deliver a CD19-specific CAR gene to the TRC1-2 recognition site. Non-edited cells were depleted using CD3 selection reagents, rendering the CAR T cells unresponsive to alloantigens. In this example, CAR T cells were produced using cells from Key Biologics donors K799 and K6366. To serve as alloantigen responders, CD3⁺ T cells were isolated from apheresis products obtained from healthy, consenting donors. CD3 isolation was performed using CD3 Positive Selection Kit II (Stem Cell Technologies). T cells were isolated from donors K6366 (Key Biologics), HC9189 (HemaCare), and OKBI4789 (Oklahoma Blood Institute). Using these five cell preparations, four different co-cultures were assembled: one autologous or syngeneic control (K6366 CD3⁺ T cells versus K6366 CAR T cells), and three allogeneic co-cultures.

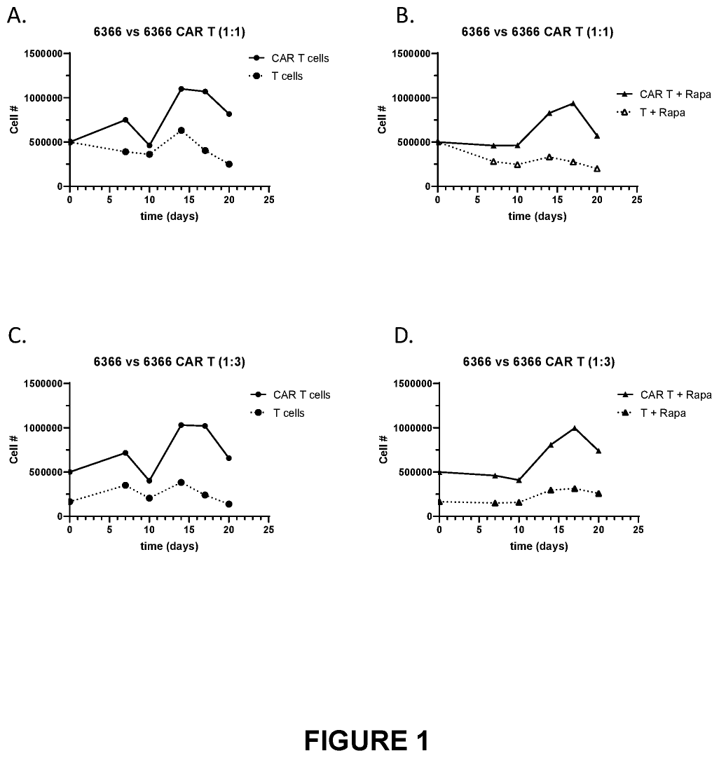

CAR T cells and allogeneic CD3⁺ T cells were plated at 1:1 or 3:1 ratios in XVIVO-15 base medium (Lonza) supplemented with 5% fetal bovine serum. Cultures were carried out in the presence of 30 ng/ml rapamycin (Selleck Chem) or in its absence beginning on the day of plating. Cultures were supplemented with 10 ng/ml interleukin-2 (IL-2, Gibco) beginning 24 hours after plating. Culture medium was refreshed, including fresh drug and cytokine, every 2-3 days. At the time points indicated in FIGS. 1-4, cultures were sampled, and the numbers of live CD3⁺ effectors and CD3⁻ targets were determined using a Beckman-Coulter CytoFLEX-S cytometer and anti-CD3-PE (clone UCHT-1, BD Biosciences), anti-CD8-FITC (clone RPA-T8, BD Biosciences), and anti-CD4-APC (clone OKT-4, BioLegend).

### 2. Results

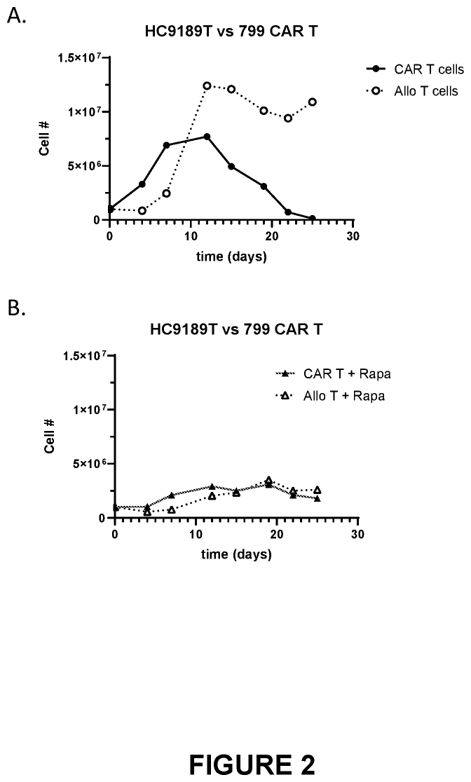

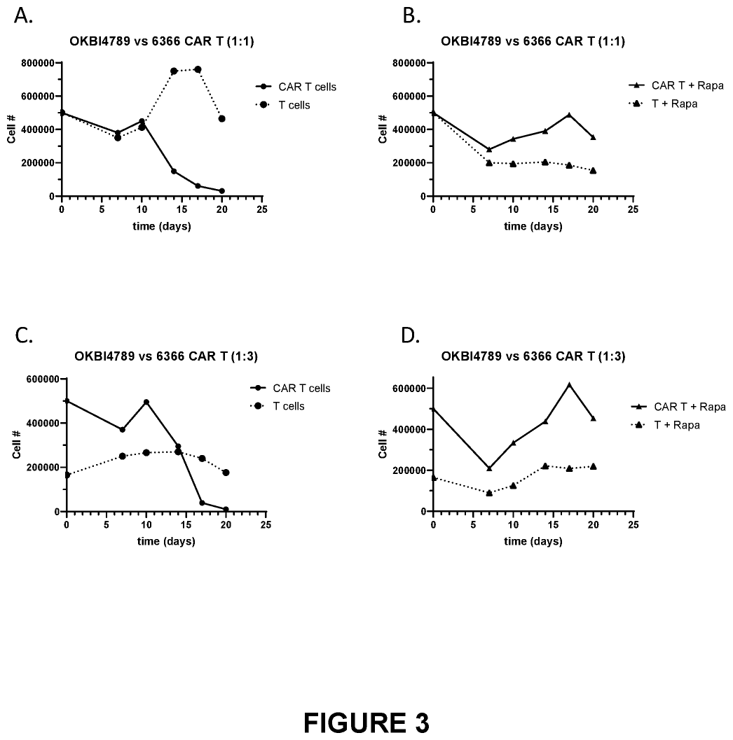

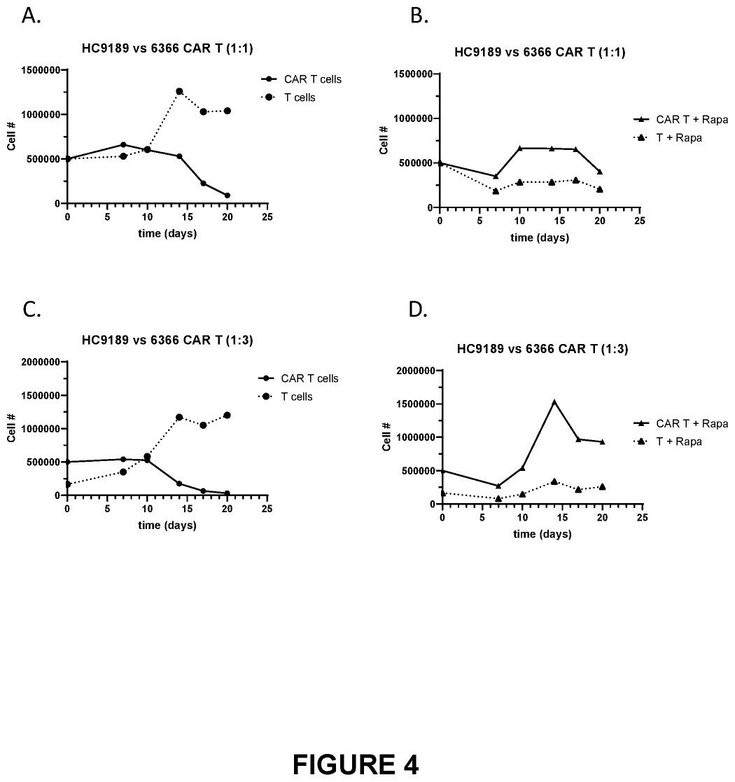

The numbers of CD3⁺ T cell effectors and CD3⁻ CAR T targets over time for the autologous co-culture appear in FIG. 1 (K6366 T cells vs. K6366 CAR T). In the absence of drug (Panels A and C), or in the presence of rapamycin (B and D) the numbers of CAR T cells did not decrease over time, and the effectors failed to expand at either E:T ratio. By comparison, allogeneic co-cultures displayed different characteristics. K799 CAR T targets underwent a transient expansion period before being reduced in number, beginning at d12 in the absence of drug (FIG. 2A). Expansion of HC9189 CD3⁺ effectors was also observed. Both the loss of CAR T targets and expansion of allogeneic T cell effectors were inhibited in the presence of 30 ng/ml rapamycin (FIG. 2B). These observations were also made on co-cultures of K6366 CAR T targets with either OKBI4789 effectors (FIG. 3) or HC9189 effectors (FIG. 4). OKBI4789 T cells expand, and CAR T target numbers begin decreasing at approximately d10 at a 1:1 E:T (FIG. 3A). At a 1:3 E:T, OKBI4789 T cells were not observed expanding, but targets were reduced in number (3C). These observations were not made in the presence of rapamycin (FIGS. 3 B and D): effector T cells did not expand, and target cell numbers did not decrease over time. Expansion of HC9189 T cells was observed at both E:T ratios (FIGS. 4A and C) in the absence of drug. As in the other allogeneic co-cultures, CAR T targets began decreasing in number at approximately d10. Rapamycin was found to inhibit the expansion of HC9189 effectors and the decrease in CAR T target numbers (FIGS. 4B and D).

### 3. Conclusion

Co-culturing TRAC-edited CAR T cells with CD3⁺ T cells from unrelated donors elicited a proliferative response from the allogeneic responders, and a decrease in CAR T numbers. This was not observed when the CD3⁺ effectors were sourced from the same donor as the CAR T targets, indicating that alloantigen responses were responsible for driving T cell proliferation and cytotoxic responses against the CAR T cells. Adding rapamycin to the cocultures prevented both features of an alloantigen response in three different mismatch cultures.

### Example 2

### Rapamycin Prevents CAR T Proliferative Responses but not Cytotoxicity Against Tumor Target Cells

### 1. Methods

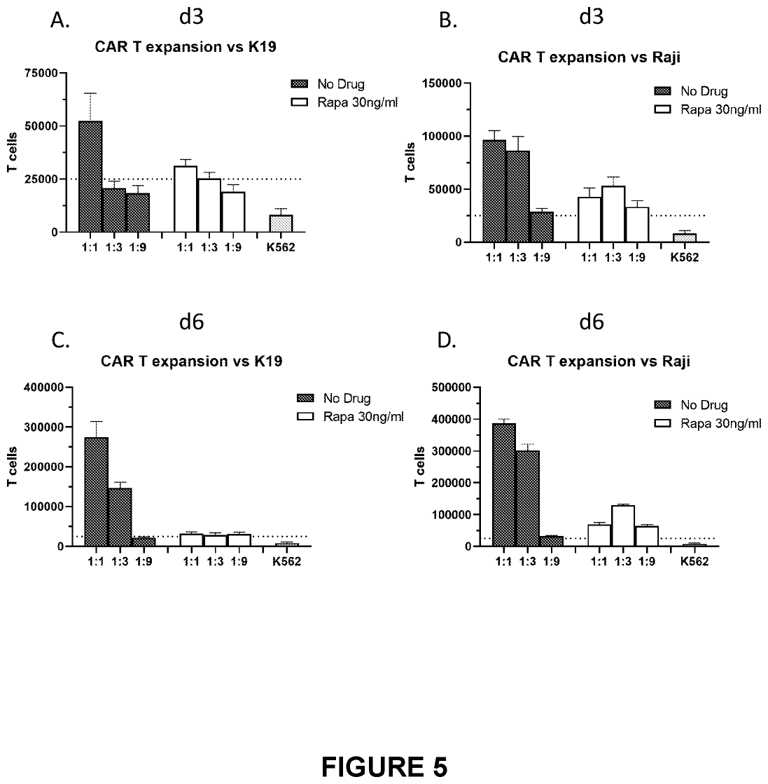

TRAC-edited CAR T cells were produced as above and were plated with the Burkitt's lymphoma line Raji (ATCC), or with the chronic myelogenous leukemia line K562 (sourced from ATCC) engineered to express CD19 (referred to herein as K19 targets). CAR T cells were cultured in XVIVO-15 medium (as above) with Raji or K19 targets at E:T ratios of 1:1, 1:3, or 1:9 in the presence or absence of 30 ng/ml rapamycin (Selleck Chem). That is 2.0e⁴ CAR T cells were plated with either 2.0e⁴, 6.0e⁴, or 1.8e⁵ target cells. No exogenous cytokine was added to the culture medium. Cultures were analyzed as in Example 1 after 3 and 6 days of co-culture. Live target and effector cells were enumerated and plotted in FIGS. 5-7.

### 2. Results

Expansion of CAR T cells in response to antigen stimulation was determined following 3 days (FIGS. 5A and B) and 6 days (C and D) of co-culture. Proliferation following K19 (FIGS. 5A and C) or Raji (B and D) was assessed. In the absence of drug, CAR T cells were present in greater numbers relative to input (horizontal dashed line in FIG. 5) at the 1:1 E:T ratio for K19 cocultures, and at the 1:1 and 1:3 ratios for Raji cocultures after 3 days of culture. CD19⁻ K562 cells did not elicit CAR T expansion. Additional increases were evident by day 6 of culture irrespective of CD19⁺ target line, with 1:1 and 1:3 ratios, but not 1:9 ratios exhibiting CART expansion in the absence of drug. CAR T cells were not found to be present in greater numbers in any co-culture at any time point in the presence of 30 ng/ml rapamycin.

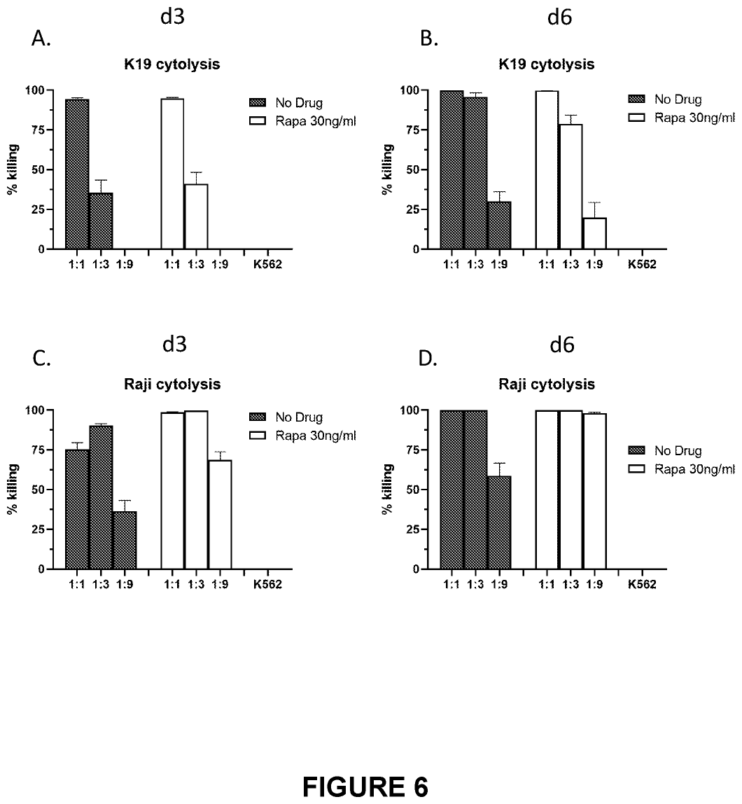

Cytolysis by CAR T cells is plotted in FIG. 6 for K19 (A and B) and Raji targets (C and D) where A and C show data collected after 3 days of culture and B and D show data collected at d6. In K19 co-cultures, extensive killing at a 1:1 ET is observed at d3 while moderate killing is observed at a 1:3 ratio and no killing is seen at 1:9. After an additional 3 days of culture, extensive killing has taken place at 1:1 and 1:3 with moderate target killing observed at 1:9. No discernable difference in K19 cytolysis was observed in the presence of rapamycin compared to the no drug controls. In similar fashion, Raji cells are nearly eliminated at 1:1 and 1:3 ratios by d3, and moderate killing is observed at 1:9. An additional 3d does not appear to increase killing at 1:9 in a meaningful way.

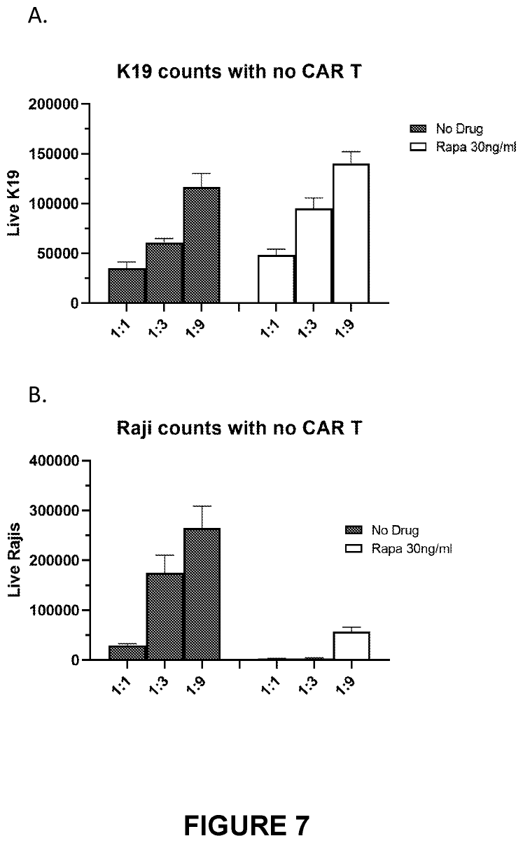

Control wells in which target cells were plated in the presence or absence of rapamycin but without CAR T cells demonstrated differences in growth/survival of the targets in the presence of drug (FIG. 7). K19 numbers at d6 of culture varied depending on the number of cells originally plated, but not with respect to drug treatment (FIG. 7A), while Raji cultures receiving rapamycin contained fewer cells at d6 than corresponding drug-free cultures (FIG. 7B).

### 3. Conclusion

Rapamycin affects CAR T activity following encounter with antigen-bearing target cells. Proliferation is profoundly inhibited, but cytolytic functions are largely unaffected. Given that the expansion of CAR T cells that is observed at 1:3 ratios is inhibited by rapamycin, the effective E:T ratio is therefore reduced by the drug, but target killing is unchanged. This suggests that rapamycin may enhance the per-cell potency of CAR T cells or prevent some of the exhaustive effects associated with robust proliferation. The observation that rapamycin is not well tolerated by Raji cells cultured in the absence of CAR T cells suggests that anti-tumor effects of CAR T cells and rapamycin may be leveraged to improve patient outcomes in lymphoma.

1. A method for reducing the number of target cells in a subject, said method comprising:
(a) administering to said subject a lymphodepletion regimen that comprises administering one or more effective doses of pentostatin and one or more effective doses of cyclophosphamide; and
(b) administering to said subject an effective dose of a pharmaceutical composition comprising a population of human immune cells, wherein a plurality of said human immune cells are genetically-modified human immune cells that express a chimeric antigen receptor (CAR) or an exogenous T cell receptor (TCR), wherein said lymphodepletion regimen is administered prior to administration of said pharmaceutical composition,
and wherein said CAR or said exogenous TCR comprises an extracellular ligand-binding domain having specificity for an antigen on said target cells.

2. The method of claim 1, wherein said lymphodepletion regimen comprises administering said cyclophosphamide at a dose of about 50 to about 500 mg/day.

3. The method of claim 1 or 2, wherein said lymphodepletion regimen comprises administering said cyclophosphamide at a dose of about 100 to about 300 mg/day.

4. The method of any one of claims 1-3, wherein said lymphodepletion regimen comprises administering said cyclophosphamide at a dose of about 200 mg/day.

5. The method of any one of claims 1-4, wherein said lymphodepletion regimen comprises administering said cyclophosphamide once daily starting 11 days and ending 1 day prior to administration of said pharmaceutical composition.

6. The method of any one of claims 1-4, wherein said lymphodepletion regimen comprises administering said cyclophosphamide once daily starting 9 days and ending 1 day prior to administration of said pharmaceutical composition.

7. The method of any one of claims 1-4, wherein said lymphodepletion regimen comprises administering said cyclophosphamide once daily starting 7 days and ending 1 day prior to administration of said pharmaceutical composition.

8. The method of any one of claims 1-7, wherein said cyclophosphamide is administered orally.

9. The method of any one of claims 1-8, wherein said lymphodepletion regimen comprises administering about 200 mg of said cyclophosphamide once daily starting 7 days and ending 1 day prior to administration of said pharmaceutical composition.

10. The method of any one of claims 1-9, wherein said lymphodepletion regimen comprises administering said pentostatin at a dose of about 1 to about 20 mg/m²/day.

11. The method of claim 10, wherein said lymphodepletion regimen comprises administering said pentostatin at a dose of about 2 to about 10 mg/m²/day.

12. The method of claim 11, wherein said lymphodepletion regimen comprises administering said pentostatin at a dose of about 4 mg/m²/day.

13. The method of any one of claims 1-12, wherein said lymphodepletion regimen comprises administering said pentostatin once daily starting 12 days and ending 1 day prior to administration of said pharmaceutical composition.

14. The method of any one of claims 1-12, wherein said lymphodepletion regimen comprises administering said pentostatin once daily starting 9 days and ending 3 days prior to administration of said pharmaceutical composition.

15. The method of any one of claims 1-12, wherein said lymphodepletion regimen comprises administering said pentostatin every 3 days starting 15 days and ending 3 days prior to administration of said pharmaceutical composition.

16. The method of any one of claims 1-12, wherein said lymphodepletion regimen comprises administering said pentostatin every 3 days starting 9 days and ending 3 days prior to administration of said pharmaceutical composition.

17. The method of any one of claims 1-16, wherein said pentostatin is administered intravenously.

18. The method of any one of claims 1-17, wherein said lymphodepletion regimen comprises administering about 4 mg/m²/day of said pentostatin to said subject every 3 days starting 9 days and ending 3 days prior to administration of said pharmaceutical composition.

19. The method of claim 18, wherein said lymphodepletion regimen comprises administering about 200 mg/day of said cyclophosphamide once daily starting 7 days and ending 1 day prior to administration of said pharmaceutical composition and administering about 4 mg/m²/day of said pentostatin every 3 days starting 9 days and ending 3 days prior to administration of said pharmaceutical composition.

20. The method of any one of claims 1-19, wherein said lymphodepletion regimen does not comprise an effective dose of a biological lymphodepletion agent.

21. The method of any one of claims 1-20, wherein said lymphodepletion regimen does not comprise administering a biological lymphodepletion agent.

22. The method of claim 20 or 21, wherein said biological lymphodepletion agent is a monoclonal antibody, or a fragment thereof.

23. The method of claim 22, wherein said monoclonal antibody, or fragment thereof, has specificity for a T cell antigen.

24. The method of claim 23, wherein said monoclonal antibody, or fragment thereof, is an anti-CD52 monoclonal antibody, or fragment thereof, or an anti-CD3 antibody, or fragment thereof.

25. The method of claim 24, wherein said monoclonal antibody is alemtuzumab or ALLO-647.

26. The method of any one of claims 1-25, wherein said method further comprises administering an effective dose of an immunosuppressant agent to said subject.

27. The method of claim 26, wherein said immunosuppressant agent comprises sirolimus.

28. The method of claim 27, wherein said effective dose of sirolimus results in an effective serum concentration of sirolimus in said subject of about 15 ng/mL to about 30 ng/mL.

29. The method of claim 27 or 28, wherein said effective dose of sirolimus results in an effective serum concentration of sirolimus in said subject of about 20 ng/mL to about 30 ng/mL.

30. The method of any one of claims 27-29, wherein said effective dose of sirolimus results in an effective serum concentration of sirolimus in said subject of about 20 ng/mL.

31. The method of any one of claims 27-30, wherein sirolimus is administered once daily starting 9 days prior to administration of said pharmaceutical composition.

32. The method of any one of claims 27-30, wherein sirolimus is administered once daily starting 5 days prior to administration of said pharmaceutical composition.

33. The method of any one of claims 27-30, wherein sirolimus is administered once daily starting 1 day prior to administration of said pharmaceutical composition.

34. The method of any one of claims 27-33, wherein sirolimus is administered once on the day of administration of said pharmaceutical composition, and once daily each day thereafter ending 28 days after administration of said pharmaceutical composition.

35. The method of any one of claims 27-33, wherein sirolimus is administered once on the day of administration of said pharmaceutical composition, and once daily each day thereafter ending 25 days after administration of said pharmaceutical composition.

36. The method of any one of claims 27-33, wherein sirolimus is administered once on the day of administration of said pharmaceutical composition, and once daily each day thereafter ending 21 days after administration of said pharmaceutical composition.

37. The method of any one of claims 27-39, wherein sirolimus is administered once daily starting 1 day prior to administration of said pharmaceutical composition and ending 21 days after administration of said pharmaceutical composition.

38. The method of any one of claims 27-37, wherein sirolimus is administered orally.

39. The method of any one of claims 27-30, wherein said subject is first administered one loading dose of sirolimus, followed by administration of a maintenance dose of sirolimus once daily in order to achieve said effective serum concentration of sirolimus, wherein said once daily maintenance dose of sirolimus begins the day after the loading dose of sirolimus is administered to said subject.

40. The method of claim 39, wherein said loading dose of sirolimus is about 5 mg to about 20 mg.

41. The method of claim 39, wherein said loading dose of sirolimus is about 10 mg to about 18 mg.

42. The method of claim 39, wherein said loading dose of sirolimus is about 16 mg.

43. The method of any one of claims 39-42, wherein said maintenance dose of sirolimus is about 1 mg to about 15 mg.

44. The method of any one of claims 39-42, wherein said maintenance dose of sirolimus is about 2 mg to about 10 mg.

45. The method of any one of claims 39-42, wherein said maintenance dose of sirolimus is about 4 mg.

46. The method of any one of claims 39-45, wherein said loading dose of sirolimus is administered orally.

47. The method of any one of claims 39-46, wherein said maintenance dose of sirolimus is administered orally.

48. The method of any one of claims 39-47, wherein said loading dose of sirolimus is administered once 8 days prior to administration of said pharmaceutical composition, and wherein said maintenance dose of sirolimus is administered once daily beginning 7 days prior to administration of said pharmaceutical composition.

49. The method of any one of claims 39-47, wherein said loading dose of sirolimus is administered once 5 days prior to administration of said pharmaceutical composition, and wherein said maintenance dose of sirolimus is administered once daily beginning 4 days prior to administration of said pharmaceutical composition.

50. The method of any one of claims 39-47, wherein said loading dose of sirolimus is administered once 1 day prior to administration of said pharmaceutical composition, and wherein said maintenance dose of sirolimus is administered once daily beginning on the same day as administration of said pharmaceutical composition.

51. The method of any one of claims 39-50, wherein said maintenance dose is administered once daily ending 28 days after administration of said pharmaceutical composition.

52. The method of any one of claims 39-50, wherein said maintenance dose is administered once daily ending 25 days after administration of said pharmaceutical composition.

53. The method of any one of claims 39-50, wherein said maintenance dose is administered once daily ending 21 days after administration of said pharmaceutical composition.

54. The method of any one of claims 39-47, wherein said loading dose of sirolimus is administered once one day prior to administration of said pharmaceutical composition, and wherein said maintenance dose of sirolimus is administered once daily beginning on the same day as administration of said pharmaceutical composition and ending 21 days after administration of said pharmaceutical composition.

55. A method for reducing the number of target cells in a subject, said method comprising:
(a) administering to said subject an immunosuppression regimen that comprises administering one or more effective doses of sirolimus; and
(b) administering to said subject an effective dose of a pharmaceutical composition comprising a population of human immune cells, wherein a plurality of said human immune cells are genetically-modified human immune cells that express a CAR or an exogenous TCR, and wherein said CAR or said exogenous TCR comprises an extracellular ligand-binding domain having specificity for an antigen on said target cells.

56. The method of claim 55, wherein said effective dose of sirolimus results in an effective serum concentration of sirolimus in said subject of about 15 ng/mL to about 30 ng/mL.

57. The method of claim 55, wherein said effective dose of sirolimus results in an effective serum concentration of sirolimus in said subject of about 20 ng/mL to about 30 ng/mL.

58. The method of claim 55, wherein said effective dose of sirolimus results in an effective serum concentration of sirolimus in said subject of about 20 ng/mL.

59. The method of any one of claims 55-58, wherein said sirolimus is administered once daily starting 9 days prior to administration of said pharmaceutical composition.

60. The method of any one of claims 55-58, wherein said sirolimus is administered once daily starting 5 days prior to administration of said pharmaceutical composition.

61. The method of any one of claims 55-58, wherein said sirolimus is administered once daily starting 1 day prior to administration of said pharmaceutical composition.

62. The method of any one of claims 55-61, wherein said sirolimus is administered on the day of administration of said pharmaceutical composition, and once daily each day thereafter ending 28 days after administration of said pharmaceutical composition.

63. The method of any one of claims 55-61, wherein said sirolimus is administered on the day of administration of said pharmaceutical composition, and once daily each day thereafter ending 25 days after administration of said pharmaceutical composition.

64. The method of any one of claims 55-61, wherein said sirolimus is administered on the day of administration of said pharmaceutical composition, and once daily each day thereafter ending 21 days after administration of said pharmaceutical composition.

65. The method of any one of claims 55-58, wherein said sirolimus is administered once daily starting 1 day prior to administration of said pharmaceutical composition and ending 21 days after administration of said pharmaceutical composition.

66. The method of any one of claims 55-65, wherein said sirolimus is administered orally.

67. The method of any one of claims 55-58, wherein said subject is first administered one loading dose of sirolimus, followed by administration of a maintenance dose of sirolimus once daily in order to achieve said effective serum concentration of sirolimus, wherein said once daily maintenance dose of sirolimus begins the day after the loading dose of sirolimus is administered to said subject.

68. The method of claim 67, wherein said loading dose of sirolimus is about 5 mg to about 20 mg.

69. The method of claim 67, wherein said loading dose of sirolimus is about 10 mg to about 18 mg.

70. The method of claim 67, wherein said loading dose of sirolimus is about 16 mg.

71. The method of any one of claims 67-70, wherein said maintenance dose of sirolimus is about 1 mg to about 15 mg.

72. The method of any one of claims 67-70, wherein said maintenance dose of sirolimus is about 2 mg to about 10 mg.

73. The method of any one of claims 67-70, wherein said maintenance dose of sirolimus is about 4 mg.

74. The method of any one of claims 67-73, wherein said loading dose of sirolimus is administered orally.

75. The method of any one of claims 67-74, wherein said maintenance dose of sirolimus is administered orally.

76. The method of any one of claims 67-75, wherein said loading dose of sirolimus is administered once 8 days prior to administration of said pharmaceutical composition, and wherein said maintenance dose of sirolimus is administered once daily beginning 7 days prior to administration of said pharmaceutical composition.

77. The method of any one of claims 67-75, wherein said loading dose of sirolimus is administered once 5 days prior to administration of said pharmaceutical composition, and wherein said maintenance dose of sirolimus is administered once daily beginning 4 days prior to administration of said pharmaceutical composition.

78. The method of any one of claims 67-75, wherein said loading dose of sirolimus is administered once 1 day prior to administration of said pharmaceutical composition, and wherein said maintenance dose of sirolimus is administered once daily beginning on the same day as administration of said pharmaceutical composition.

79. The method of any one of claims 67-78, wherein said maintenance dose is administered once daily ending 28 days after administration of said pharmaceutical composition.

80. The method of any one of claims 67-78, wherein said maintenance dose is administered once daily ending 25 days after administration of said pharmaceutical composition.

81. The method of any one of claims 67-78, wherein said maintenance dose is administered once daily ending 21 days after administration of said pharmaceutical composition.

82. The method of any one of claims 67-75, wherein said loading dose of sirolimus is administered once one day prior to administration of said pharmaceutical composition, and wherein said maintenance dose of sirolimus is administered once daily beginning on the same day as administration of said pharmaceutical composition and ending 21 days after administration of said pharmaceutical composition.

83. The method of any one of claims 55-82, wherein said method further comprises administering to said subject a lymphodepletion regimen, wherein said lymphodepletion regimen includes one or more effective doses of at least one lymphodepletion agent, and wherein said lymphodepletion regimen is administered prior to administration of said pharmaceutical composition.

84. The method of claim 83, wherein said one or more lymphodepletion agents comprises cyclophosphamide.

85. The method of claim 83 or 84, wherein said one or more lymphodepletion agents comprises pentostatin.

86. The method of any one of claims 83-85, wherein said lymphodepletion regimen comprises one more effective doses of cyclophosphamide and one or more effective doses of pentostatin.

87. The method of any one of claims 83-86, wherein said one or more lymphodepletion agents comprises fludarabine.

88. The method of any one of claims 83-87, wherein said lymphodepletion regimen comprises one more effective doses of cyclophosphamide and one or more effective doses of fludarabine.

89. The method of any one of claims 83-88, wherein said lymphodepletion regimen does not comprise an effective dose of a biological lymphodepletion agent.

90. The method of any one of claims 83-89, wherein said lymphodepletion regimen does not comprise administering a biological lymphodepletion agent.

91. The method of claim 90, wherein said biological lymphodepletion agent is a monoclonal antibody, or a fragment thereof.

92. The method of claim 91, wherein said monoclonal antibody, or fragment thereof, has specificity for a T cell antigen.

93. The method of claim 92, wherein said monoclonal antibody, or fragment thereof, is an anti-CD52 monoclonal antibody, or fragment thereof, or an anti-CD3 antibody, or fragment thereof.

94. The method of claim 93, wherein said monoclonal antibody is alemtuzumab or ALLO-647.

95. The method of any one of claims 1-94, wherein said pharmaceutical composition is administered at a dose of between about 1×10⁴ and about 1×10⁸ genetically-modified human immune cells/kg.

96. The method of any one of claims 1-94, wherein said pharmaceutical composition is administered at a dose of between about 1×10⁵ and about 1×10⁷ genetically-modified human immune cells/kg.

97. The method of any one of claims 1-94, wherein said pharmaceutical composition is administered at a dose of between about 1×10⁵ and about 6×10⁶ genetically-modified human immune cells/kg.

98. The method of any one of claims 1-94, wherein said pharmaceutical composition is administered at a dose of between about 3×10⁵ and about 6×10⁶ genetically-modified human immune cells/kg.

99. The method of any one of claims 1-94, wherein said pharmaceutical composition is administered at a dose of between about 3×10⁵ and about 3×10⁶ genetically-modified human immune cells/kg.

100. The method of any one of claims 1-94, wherein said pharmaceutical composition is administered at a dose of about 3×10⁶ genetically-modified human immune cells/kg.

101. The method of any one of claims 1-100, wherein said effective dose of said pharmaceutical composition comprises no more than 3×10⁸ genetically-modified human immune cells.

102. The method of any one of claims 1-101, wherein said method further comprises administering a second dose of said pharmaceutical composition to said subject.

103. The method of any one of claims 1-102, wherein said human immune cells are derived from said subject.

104. The method of any one of claims 1-102, wherein said human immune cells are not derived from said subject.

105. The method of any one of claims 1-104, wherein said human immune cells comprise human T cells, or cells derived therefrom, or human natural killer (NK) cells, or cells derived therefrom.

106. The method of claim 105, wherein said human immune cells comprise human T cells.

107. The method of any one of claims 1-106, wherein said target cells are cancer cells.

108. The method of claim 107, wherein said cancer cells are from a cancer of B cell origin or multiple myeloma.

109. The method of claim 108, wherein said cancer of B cell origin is acute lymphoblastic leukemia (ALL), chronic lymphocytic leukemia (CLL), small lymphocytic lymphoma (SLL), or non-Hodgkin lymphoma (NHL).

110. The method of claim 109, wherein said NHL is mantle cell lymphoma (MCL) or diffuse large B cell lymphoma (DLBCL).

111. The method of any one of claims 1-110, wherein said subject is refractory to prior CAR T immunotherapy.

112. The method of any one of claims 1-111, wherein said genetically-modified human immune cells comprise an inactivated TCR alpha gene or an inactivated TCR beta gene.

113. The method of claim 112, wherein a transgene encoding said CAR or said exogenous TCR is inserted into the genome of said genetically-modified human immune cells within said TCR alpha gene or said TCR beta gene, wherein said transgene disrupts expression of said TCR alpha gene or said TCR beta gene.

114. The method of claim 113, wherein said transgene encoding said CAR or said exogenous TCR is inserted into a TCR alpha constant region gene.

115. The method of claim 114, wherein said transgene encoding said CAR or said exogenous TCR is inserted into an engineered meganuclease recognition sequence comprising SEQ ID NO: 1 within said TCR alpha constant region gene.

116. The method of claim 115, wherein said transgene encoding said CAR or said exogenous TCR is inserted between positions 13 and 14 of SEQ ID NO: 1 within said TCR alpha constant region gene.

117. The method of any one of claims 1-116, wherein said genetically-modified human immune cells do not have detectable cell surface expression of an endogenous alpha/beta TCR.

118. The method of any one of claims 1-117, wherein said genetically-modified human immune cells do not have detectable cell surface expression of CD3.

119. The method of any one of claims 1-118, wherein said extracellular ligand-binding domain has specificity for CD19, CD20, or B cell maturation antigen (BCMA).

120. The method of any one of claims 1-119, wherein said genetically-modified human immune cell comprises a CAR, and wherein said extracellular ligand-binding domain comprises a single-chain variable fragment (scFv).

121. The method of claim 120, wherein said extracellular ligand-binding domain comprises an scFv comprising:
(a) a heavy chain variable domain (VH) of SEQ ID NO: 3 and a light chain variable domain (VL) of SEQ ID NO: 4; or
(b) a heavy chain variable domain (VH) of SEQ ID NO: 6 and a light chain variable domain (VL) of SEQ ID NO: 7.

122. The method of any one of claims 1-121, wherein said CAR comprises a CD8 alpha hinge domain.

123. The method of any one of claims 1-122, wherein said CAR comprises a CD8 alpha transmembrane domain.

124. The method of any one of claims 1-123, wherein said CAR comprises a co-stimulatory domain comprising one or more TRAF-binding domains.

125. The method of any one of claims 1-124, wherein said CAR comprises a co-stimulatory domain comprising a first domain comprising SEQ ID NO: 9 and a second domain comprising SEQ ID NO: 10 or 11.

126. The method of any one of claims 1-125, wherein said CAR comprises a novel 6 (N6) co-stimulatory domain or a 4-1BB co-stimulatory domain.

127. The method of any one of claims 1-126, wherein said CAR comprises CD3 zeta intracellular signaling domain.

128. The method of any one of claims 1-127, wherein said CAR comprises an amino acid sequence having at least 80% sequence identity to SEQ ID NO: 5 and has specificity for CD19, or an amino acid sequence having at least 80% sequence identity to SEQ ID NO: 8 and has specificity for CD20.

129. The method of claim 128, wherein said CAR comprises an amino acid sequence of SEQ ID NO: 5 or 8.

130. The method of any one of claims 1-129, wherein said genetically-modified human immune cells represent between about 40% and about 75% of said human immune cells in said population.

131. The method of any one of claims 1-129, wherein said genetically-modified human immune cells represent between about 50% and about 70% of said human immune cells in said population.

132. The method of any one of claims 1-131, wherein said genetically-modified human immune cells proliferate in vivo for at least one day following administration of said pharmaceutical composition.

133. The method of claim 132, wherein said genetically-modified human immune cells proliferate in vivo between about day 1 and about day 21 following administration of said pharmaceutical composition.

134. The method of any one of claims 1-133, wherein the number of copies of said CAR or said exogenous TCR transgene per μg of DNA in peripheral blood mononuclear cells is elevated for up to 21 days after administration of said pharmaceutical composition when compared to the number of copies present prior to administration.

135. The method of any one of claims 1-134, wherein the serum concentration of C-reactive protein, ferritin, IL-6, interferon gamma, or any combination thereof, is elevated compared to the concentration at day 0 for at least 1 day following administration of said pharmaceutical composition.

136. The method of any one of claims 1-135, wherein said method is an immunotherapy for the treatment of a disease, such as cancer, and wherein said subject achieves a partial response or a complete response to said method of immunotherapy.

137. The method of claim 136, wherein said partial response or said complete response is maintained through at least 28 days after administration of said pharmaceutical composition.

138. The method of claim 1, wherein said target cells are ALL, MCL, or DLBCL cells,
wherein said lymphodepletion regimen comprises administering:
a) cyclophosphamide at a dose of about 200 mg/day to said subject once daily starting 7 days and ending 1 day prior to administration of said pharmaceutical composition; and
b) pentostatin at a dose of about 4 mg/m²/day to said subject once every 3 days starting 9 days and ending 3 days prior to administration of said pharmaceutical composition;
wherein said genetically-modified human immune cells are CAR T cells,
wherein said pharmaceutical composition is administered at a dose of between about 3×10⁵ and 3×10⁶ CAR T cells/kg and not to exceed 3×10⁸ CAR T cells,
wherein a transgene encoding said CAR is inserted into a TCR alpha constant region gene, and
wherein said CAR comprises an scFv having specificity for CD19, a CD8 alpha hinge domain, a CD8 alpha transmembrane domain, a co-stimulatory domain comprising one or more TRAF-binding domains, and a CD3 zeta intracellular signaling domain.

139. The method of claim 1, wherein said target cells are ALL, MCL, or DLBCL cells, wherein said lymphodepletion regimen comprises administering:
a) cyclophosphamide at a dose of about 200 mg/day to said subject once daily starting 7 days and ending 1 day prior to administration of said pharmaceutical composition; and
b) pentostatin at a dose of about 4 mg/m²/day to said subject once every 3 days starting 9 days and ending 3 days prior to administration of said pharmaceutical composition;
wherein said genetically-modified human immune cells are CAR T cells,
wherein said pharmaceutical composition is administered at a dose of between about 3×10⁵ and 3×10⁶ CAR T cells/kg and not to exceed 3×10⁸ CAR T cells,
wherein a transgene encoding said CAR is inserted into a TCR alpha constant region gene, and
wherein said CAR comprises an scFv comprising a VH domain of SEQ ID NO: 3 and a VL domain of SEQ ID NO: 4, a CD8 alpha hinge domain, a CD8 alpha transmembrane domain, an N6 co-stimulatory domain, and a CD3 zeta intracellular signaling domain.

140. The method of claim 1, wherein said target cells are NHL, CLL, or SLL cells,
wherein said lymphodepletion regimen comprises administering:
a) cyclophosphamide at a dose of about 200 mg/day to said subject once daily starting 7 days and ending 1 day prior to administration of said pharmaceutical composition; and
b) pentostatin at a dose of about 4 mg/m²/day to said subject once every 3 days starting 9 days and ending 3 days prior to administration of said pharmaceutical composition;
wherein said genetically-modified human immune cells are CAR T cells,
wherein said pharmaceutical composition is administered at a dose of between about 3×10⁵ and 3×10⁶ CAR T cells/kg and not to exceed 3×10⁸ CAR T cells,
wherein a transgene encoding said CAR is inserted into a TCR alpha constant region gene, and
wherein said CAR comprises an scFv having specificity for CD20, a CD8 alpha hinge domain, a CD8 alpha transmembrane domain, a co-stimulatory domain comprising one or more TRAF-binding domains, and a CD3 zeta intracellular signaling domain.

141. The method of claim 1, wherein said target cells are NHL, CLL, or SLL cells, wherein said lymphodepletion regimen comprises administering:
a) cyclophosphamide at a dose of about 200 mg/day to said subject once daily starting 7 days and ending 1 day prior to administration of said pharmaceutical composition; and
b) pentostatin at a dose of about 4 mg/m²/day to said subject once every 3 days starting 9 days and ending 3 days prior to administration of said pharmaceutical composition;
wherein said genetically-modified human immune cells are CAR T cells,
wherein said pharmaceutical composition is administered at a dose of between about 3×10⁵ and 3×10⁶ CAR T cells/kg and not to exceed 3×10⁸ CAR T cells,
wherein a transgene encoding said CAR is inserted into a TCR alpha constant region gene, and
wherein said CAR comprises an scFv comprising a VH domain of SEQ ID NO: 6 and a VL domain of SEQ ID NO: 7, a CD8 alpha hinge domain, a CD8 alpha transmembrane domain, an N6 co-stimulatory domain, and a CD3 zeta intracellular signaling domain.

142. The method of claim 1, wherein said target cells are multiple myeloma cells, wherein said lymphodepletion regimen comprises administering:
a) cyclophosphamide at a dose of about 200 mg/day to said subject once daily starting 7 days and ending 1 day prior to administration of said pharmaceutical composition; and
b) pentostatin at a dose of about 4 mg/m²/day to said subject once every 3 days starting 9 days and ending 3 days prior to administration of said pharmaceutical composition;
wherein said genetically-modified human immune cells are CAR T cells,
wherein said pharmaceutical composition is administered at a dose of between about 3×10⁵ and 3×10⁶ CAR T cells/kg and not to exceed 3×10⁸ CAR T cells,
wherein a transgene encoding said CAR is inserted into a TCR alpha constant region gene, and
wherein said CAR comprises an scFv having specificity for BCMA, a CD8 alpha hinge domain, a CD8 alpha transmembrane domain, a co-stimulatory domain comprising one or more TRAF-binding domains, and a CD3 zeta intracellular signaling domain.

143. The method of claim 1, wherein said target cells are multiple myeloma cells, wherein said lymphodepletion regimen comprises administering:
a) cyclophosphamide at a dose of about 200 mg/day to said subject once daily starting 7 days and ending 1 day prior to administration of said pharmaceutical composition; and
b) pentostatin at a dose of about 4 mg/m²/day to said subject once every 3 days starting 9 days and ending 3 days prior to administration of said pharmaceutical composition;
wherein said genetically-modified human immune cells are CAR T cells,
wherein said pharmaceutical composition is administered at a dose of between about 3×10⁵ and 3×10⁶ CAR T cells/kg and not to exceed 3×10⁸ CAR T cells,
wherein a transgene encoding said CAR is inserted into a TCR alpha constant region gene, and
wherein said CAR comprises an scFv comprising a VH domain and a VL domain of a BCMA-specific monoclonal antibody, a CD8 alpha hinge domain, a CD8 alpha transmembrane domain, an N6 co-stimulatory domain, and a CD3 zeta intracellular signaling domain.

144. The method of any one of claims 138-143, wherein said method further comprises administering an effective dose of sirolimus to said subject that results in an effective serum concentration of sirolimus in said subject of about 20 ng/mL, wherein one loading dose of about 16 mg of sirolimus is administered one day prior to administration of said pharmaceutical composition and a maintenance dose of about 4 mg of sirolimus is administered once daily starting on the day the pharmaceutical composition is administered and ending 21 days after administration of the pharmaceutical composition.

145. The method of any one of claims 1-144, wherein said method further comprises manufacturing said population of human immune cells, wherein said manufacturing comprises:
(a) a first culturing step wherein isolated human immune cells are cultured in media for 3 days with anti-CD3 and anti-CD28 antibodies bound to a matrix or particle;
(b) electroporating said isolated human immune cells to introduce mRNA encoding an engineered nuclease having specificity for a recognition sequence within said TCR alpha gene, wherein said engineered nuclease is expressed in said human immune cells and generates a cleavage site at said recognition sequence;
(c) transducing said isolated human immune cells with a recombinant AAV vector comprising a donor template, wherein said donor template comprises a transgene encoding said CAR or said exogenous TCR, and wherein said donor template is flanked by a 5′ homology arm having homology to sequences 5′ upstream of said cleavage site, and by a 3′ homology arm having homology to sequences 3′ downstream of said cleavage site, wherein said donor template is inserted into the genome of said isolated human immune cells at said cleavage site;
(d) a second culturing step wherein said isolated human immune cells are cultured in media for about 5 days;
(e) removing said isolated human immune cells that express cell surface CD3 using anti-CD3 antibodies; and
(f) a third culturing step wherein said isolated human immune cells are cultured in media to generate said population of human immune cells.

146. The method of claim 145, wherein said manufacturing is completed in about 10 days or less.

147. The method of claim 145 or 146, wherein said engineered nuclease is an engineered meganuclease, a zinc finger nuclease, a TALEN, a compact TALEN, a CRISPR system nuclease, or a megaTAL.

148. The method of claim 147, wherein said engineered nuclease is an engineered meganuclease.

149. The method of claim 148, wherein said engineered meganuclease has specificity for a recognition sequence comprising SEQ ID NO: 1.

150. The method of claim 148 or 149, wherein said engineered meganuclease comprises an amino acid sequence of SEQ ID NO: 17.
